(19) United States
(12) Patent Application Publication
(10) Pub. No.: US 2022/0096423 A1
(43) Pub. Date: Mar. 31, 2022

(54) Treatment of Bladder Cancer by Local Administration of Taxane Particles

(71) Applicant: CRITITECH, INC., Lawrence, KS (US)

(72) Inventors: Gere S. DIZEREGA, Lawrence, KS (US); Michael BALTEZOR, Lawrence, KS (US); Charles DECEDUE, Lawrence, KS (US); Sam CAMPBELL, Lawrence, KS (US); Matthew MCCLOREY, Lawrence, KS (US)

(21) Appl. No.: 17/492,231

(22) Filed: Oct. 1, 2021

Related U.S. Application Data
(63) Continuation of application No. 16/239,527, filed on Jan. 4, 2019, now abandoned.
(60) Provisional application No. 62/614,064, filed on Jan. 5, 2018.
(60) Provisional application No. 62/779,317, filed on Dec. 13, 2018.
(60) Provisional application No. 62/678,470, filed on May 31, 2018.
(60) Provisional application No. 62/740,489, filed on Oct. 3, 2018.
(60) Provisional application No. 62/779,327, filed on Dec. 13, 2018.
(60) Provisional application No. 62/740,501, filed on Oct. 3, 2018.
(60) Provisional application No. 62/779,320, filed on Dec. 13, 2018.

(51) Int. Cl. A61K 31/337; A61K 47/26; A61K 9/16; A61P 35/00; A61K 9/00; A61K 9/08; A61K 9/10; A61K 9/14

(52) U.S. Cl. CPC A61K 31/337; A61K 47/26; A61K 9/16; A61P 35/00; A61K 9/14; A61K 9/0034; A61K 9/08; A61K 9/10; A61K 9/0019

(57) ABSTRACT

Disclosed herein are methods for treating and inhibiting the recurrence of bladder cancer by local administration of compositions comprising taxane particles such as docetaxel particles. Administration methods include intratumoral injection, direct injection into surgical tumor resection sites, and intravesical instillation.

### CROSS REFERENCE

This application claims priority to U.S. Provisional Patent Application Ser. Nos. 62/614,064 filed Jan. 5, 2018; 62/779,317 filed Dec. 13, 2018; 62/678,470 filed May 31, 2018; 62/740,489 filed Oct. 3, 2018; 62/779,327 filed Dec. 13, 2018; 62/740,501 filed Oct. 3, 2018; and 62/779,320 filed Dec. 13, 2018; each incorporated by reference herein in their entirety

### FIELD

The present disclosure generally relates to treatment of bladder cancer.

### BACKGROUND

Bladder cancer is the most common cancer in the urinary tract and accounts for about 5% of all new cancers in the US. Bladder cancer is the sixth most common cancer in the United States, with an estimated 79,030 new cases and 6,870 deaths from the disease predicted in 2017. Most patients (approximately 75%) with bladder cancer are diagnosed with disease confined to the mucosa or submucosa, classified as Non-muscle invasive bladder cancer (NMIBC). NMIBC is further stratified as low, intermediate or high-risk. Most patients with high-risk NMIBC are treated with transurethral resection of the bladder tumor (TURBT) followed by intravesical chemotherapy. The three therapies currently approved by FDA for intravesical use, bacillus Calmette-Guérin (BCG), Valrubicin, and thiotepa, are imperfect. Many patients do not respond to treatment, do not achieve a lasting response, and/or encounter serious treatment-related toxicities. In some cases, the bladder cancer often recurs following these surgeries, many times as a higher-grade tumor, resulting in the need for further treatment and surgeries including partial or radical cystectomy. Intravenous (IV) administration of chemotherapeutic agents for treatment of bladder cancer can cause systemic toxicities such as peripheral neuropathy and weight loss.

Muscle invasive bladder cancer (MIBC) is associated with a high rate of recurrence and poor overall prognosis despite aggressive local and systemic therapies. For decades, radical cystectomy has been the mainstay of treatment for muscle invasive bladder cancer. Despite providing excellent local control, surgery alone does not result in optimal survival rates. Further, radical cystectomy is associated with considerable morbidity and mortality, as well as notable long-term complications and negative impacts on quality of life.

### SUMMARY

The present disclosure provides solutions to the aforementioned limitations and deficiencies in the art relating to treatment of bladder cancer. Disclosed herein are compositions and methods for treating bladder cancer as well as for inhibiting the recurrence of bladder cancer after surgical tumor resection.

In one aspect disclosed herein is a method of treating bladder cancer or inhibiting the recurrence of bladder cancer in a subject, the method comprising: directly injecting an effective amount of a first composition comprising taxane particles into one or more bladder tumor surgical resection sites, wherein the injecting is done following surgical resection of one or more bladder tumors of the subject, wherein the taxane particles have a mean particle size (number) of from 0.1 microns to 5 microns, thereby treating or inhibiting the recurrence of the bladder cancer. In some embodiments, the method further comprises: a first (initial) instilling via intravesical instillation of an effective amount of a second composition comprising a taxane solution or taxane particles having a mean particle size (number) of from 0.1 microns to 5 microns into the bladder of the subject after injecting the first composition. In some embodiments, the method still further comprises: instilling via intravesical instillation of an effective amount of the second composition into the bladder of the subject an additional 1 to 14 times after the first (initial) instilling. In some embodiments, the instillations are separated by periodic intervals, such as about a week, about 2 weeks, about 3 weeks, about a month, about 2 months, or about 3 months. In some embodiments, the taxane particles have a mean particle size (number) of from 0.1 microns to 1.5 microns, or from 0.4 microns to 1.2 microns. In some embodiments, the taxane particles are docetaxel particles. In some embodiments, the docetaxel particles have a specific surface area (SSA) of at least 18 m²/g. In some embodiments, the docetaxel particles have a bulk density (not-tapped) of 0.05 g/cm³ to 0.15 g/cm³. In some embodiments, the taxane solution is docetaxel solution. In some embodiments, the bladder cancer does not recur for at least 3 months, or at least 6 months, or at least 12 months after the surgical resection of the one or more bladder tumors. In some embodiments, the bladder cancer is intermediate risk or high-risk bladder cancer.

In another aspect disclosed herein is a method for inhibiting the recurrence of bladder cancer in a subject who has had one or more bladder tumors surgically resected, the method comprising: (a) following surgical resection of the one or more bladder tumors, directly injecting an effective amount of a first composition comprising taxane particles into the resection site(s), wherein the taxane particles have a mean particle size (number) of from 0.1 microns to 5 microns; (b) a first (initial) instilling via intravesical instillation of an effective amount of a second composition comprising a taxane solution or taxane particles having a mean particle size (number) of from 0.1 microns to 5 microns into the bladder of the subject after injecting the first composition; and (c) instilling via intravesical instillation of an effective amount of the second composition into the bladder of the subject an additional 1-14 times after the first (initial) instilling; wherein the bladder cancer does not recur in the subject for at least 3 months, or at least 6 months, or at least 12 months after the after the surgical resection of the one or more tumors, thereby inhibiting the recurrence of the bladder cancer. In some embodiments, the instillations are separated by periodic intervals, such as about a week, about 2 weeks, about 3 weeks, about a month, about 2 months, or about 3 months. In some embodiments, the taxane particles have a mean particle size (number) of from 0.1 microns to 1.5 microns, or from 0.4 microns to 1.2 microns. In some embodiments, the taxane particles are docetaxel particles. In some embodiments, the docetaxel particles have a specific surface area (SSA) of at least 18 m²/g. In some embodiments, the docetaxel particles have a bulk density (not-tapped) of 0.05 g/cm³ to 0.15 g/cm³. In some embodiments, the taxane solution is docetaxel solution. In some embodiments, the bladder cancer was intermediate risk or high-risk bladder cancer prior to the surgical resection of the one or more bladder tumors.

In another aspect disclosed herein is a method of treating bladder cancer in a subject, the method comprising: (a) administering a first administration (first cycle) of an effective amount of a composition comprising taxane particles to a bladder tumor of the subject via intratumoral injection, wherein the taxane particles have a mean particle size (number) of from 0.1 microns to 5 microns, (b) optionally, administering a second administration (second cycle) of an effective amount of the composition to the bladder tumor via intratumoral injection within a periodic interval following the first administration in (a), and (c) optionally, administering a third administration (third cycle) of an effective amount of the composition to the bladder tumor via intratumoral injection within a periodic interval following the second administration in (b), thereby treating the bladder cancer. In some embodiments, the method further comprises administering one or more additional administrations of the composition to the bladder tumor via intratumoral injection within a periodic interval after each administration. In some embodiments, the periodic interval is about a week, about 2 weeks, about 3 weeks, about a month, about 2 months, or about 3 months. In some embodiments, the taxane particles have a mean particle size (number) of from 0.1 microns to 1.5 microns, or from 0.4 microns to 1.2 microns. In some embodiments, the taxane particles are docetaxel particles. In some embodiments, the docetaxel particles have a specific surface area (SSA) of at least 18 m²/g. In some embodiments, the docetaxel particles have a bulk density (not-tapped) of 0.05 g/cm³ to 0.15 g/cm³. In some embodiments, the bladder cancer is a low risk bladder cancer. In other embodiments, the bladder cancer is intermediate risk or high-risk bladder cancer.

In still another aspect disclosed herein is a method of administering a tumoricidal dose of a composition comprising taxane particles to a bladder tumor of a subject who has bladder cancer, the method comprising: (a) administering a first administration (first cycle) of an effective amount of the composition comprising taxane particles to the bladder tumor of the subject via intratumoral injection, wherein the taxane particles have a mean particle size (number) of from 0.1 microns to 5 microns, and (b) administering a second administration (second cycle) of an effective amount of the composition to the bladder tumor via intratumoral injection within a periodic interval following the first administration in (a), and (c) optionally, administering a third administration (third cycle) of an effective amount of the composition to the bladder tumor via intratumoral injection within a periodic interval following the second administration in (b), wherein the bladder tumor is eliminated. In some embodiments, the periodic interval is about a week, about 2 weeks, about 3 weeks, about a month, about 2 months, or about 3 months. In some embodiments, the taxane particles have a mean particle size (number) of from 0.1 microns to 1.5 microns, or from 0.4 microns to 1.2 microns. In some embodiments, the taxane particles are docetaxel particles. In some embodiments, the docetaxel particles have a specific surface area (SSA) of at least 18 m²/g. In some embodiments, the docetaxel particles have a bulk density (not-tapped) of 0.05 g/cm³ to 0.15 g/cm³. In some embodiments, the bladder cancer is a low risk bladder cancer. In other embodiments, the bladder cancer is intermediate risk or high-risk bladder cancer.

Another aspect is that the methods of the disclosure also allow for exposure of the taxane particles to a bladder tumor after administration of the composition for a sustained amount of time sufficient to stimulate the endogenous immune system of the subject resulting in the production of tumoricidal cells and infiltration of the tumoricidal cells in and/or around the tumor site at a level sufficient to treat the tumor. In some embodiments, the stimulation of the endogenous immune systems produces a cellular (cell-mediated) immune response. In other embodiments, the stimulation of the endogenous immune system produces a humoral immune response. In some embodiments, metastases are reduced or eliminated. In some embodiments, the tumoricidal cells comprise dendritic cells, macrophages, T-cells, B-cells, lymphocytes, or natural killer (NK) cells, or combinations thereof. In some embodiments, the exposure time is at least 4 weeks. In some embodiments, the sustained amount of exposure time is at least 108, 120, 132, 144, 156, 168, 180, 192, 204, 216, 228, 240, 252, 264, 276, 288, 300, 312, 324, or 336 hours. In various further embodiments, the sustained amount of exposure time is at least 3, 4, 5, 6, 7, or 8 weeks.

Also, disclosed herein are the following embodiments 1 to 94:

Embodiment 1. A method of treating bladder cancer or inhibiting the recurrence of bladder cancer in a subject, the method comprising: directly injecting an effective amount of a first composition comprising taxane particles into one or more bladder tumor surgical resection sites, wherein the injecting is done following surgical resection of one or more bladder tumors of the subject, wherein the taxane particles have a mean particle size (number) of from 0.1 microns to 5 microns, thereby treating or inhibiting the recurrence of the bladder cancer.
Embodiment 2. The method of embodiment 1, wherein the method further comprises: a first (initial) instilling via intravesical instillation of an effective amount of a second composition comprising a taxane solution or taxane particles having a mean particle size (number) of from 0.1 microns to 5 microns into the bladder of the subject after injecting the first composition.
Embodiment 3. The method of embodiment 2, wherein the method further comprises: instilling via intravesical instillation of an effective amount of the second composition into the bladder of the subject an additional 1 to 14 times after the first (initial) instilling.
Embodiment 4. The method of embodiment 3, wherein the instillations are separated by periodic intervals, such as about a week, about 2 weeks, about 3 weeks, about a month, about 2 months, or about 3 months.
Embodiment 5. The method of any one of embodiments 1 to 4, wherein the taxane particles of the first composition have a mean particle size (number) of from 0.1 microns to 1.5 microns, or from 0.4 microns to 1.2 microns, wherein the second composition comprises taxane particles, and wherein the taxane particles of the second composition have a mean particle size (number) of from 0.1 microns to 1.5 microns, or from 0.4 microns to 1.2 microns.
Embodiment 6. The method of any one of embodiments 1 to 5, wherein the taxane particles comprise at least 95% of the taxane.
Embodiment 7. The method of any one of embodiments 1 to 6, wherein the taxane particles of the first composition are docetaxel particles, wherein the second composition comprises taxane particles, and wherein the taxane particles of the second composition are docetaxel particles.
Embodiment 8. The method of embodiment 7, wherein the docetaxel particles have a specific surface area (SSA) of at least 18 m2/g.
Embodiment 9. The method of any one of embodiments 7 or 8, wherein the docetaxel particles have a bulk density (not-tapped) of 0.05 g/cm3 to 0.15 g/cm3.
Embodiment 10. The method of any one of embodiments 2 or 3, wherein the second composition comprises a taxane solution, and wherein the taxane solution is docetaxel solution.
Embodiment 11. The method of any one of embodiments 1 to 10, wherein the first composition and/or the second composition exclude albumin.
Embodiment 12. The method of any one of embodiments 1 to 11, wherein the first composition further comprises a liquid carrier, wherein the first composition comprises a suspension of the taxane particles dispersed in the liquid carrier, wherein the second composition comprises taxane particles, wherein the second composition further comprises a liquid carrier, and wherein the second composition comprises a suspension of the taxane particles dispersed in the liquid carrier.
Embodiment 13. The method of embodiment 12, wherein the liquid carrier is an aqueous carrier.
Embodiment 14. The method of embodiment 13, wherein the aqueous carrier comprises normal saline solution.
Embodiment 15. The method of any one of embodiments 13 or 14, wherein the aqueous carrier comprises a surfactant and/or ethanol.
Embodiment 16. The method of embodiment 15, wherein the aqueous carrier comprises a surfactant, and wherein the surfactant is a polysorbate.
Embodiment 17. The method of embodiment 16, wherein the polysorbate is polysorbate 80, and wherein the polysorbate 80 is present in the liquid carrier at a concentration of about 0.01% w/v to about 1% w/v.
Embodiment 18. The method of any one of embodiments 15 to 17, wherein aqueous carrier comprises ethanol, and wherein the ethanol is present at a concentration of about 0.1% w/v to about 8% w/v.
Embodiment 19. The method of any one of embodiments 12 to 18, wherein the first composition further comprises a diluent, wherein the liquid carrier and the diluent form a mixture, wherein the first composition is a suspension of the taxane particles dispersed in the liquid carrier/diluent mixture, wherein the second composition comprises taxane particles, wherein the second composition further comprises a diluent, wherein the liquid carrier and the diluent form a mixture, and wherein the second composition is a suspension of the taxane particles dispersed in the liquid carrier/diluent mixture.
Embodiment 20. The method of embodiment 19, wherein the diluent is a normal saline solution.
Embodiment 21. The method of any one of embodiments 7 to 20, wherein the concentration of the docetaxel particles in the first composition is about 1 mg/mL to about 4 mg/mL.
Embodiment 22. The method of any one of embodiment 7 to 20, wherein the second composition comprises docetaxel particles, wherein the concentration of the docetaxel particles in the second composition is about 1 mg/mL to about 15 mg/mL.
Embodiment 23. The method of any one of embodiments to 2 to 22, wherein the instillation volume of the second composition is about 25 mL.
Embodiment 24. The method of any one of embodiments 1 to 23, wherein the bladder cancer is non-muscle invasive bladder cancer (NMIBC) or muscle invasive bladder cancer (MIBC).
Embodiment 25. The method of any one of embodiments 1 to 24, wherein the bladder cancer does not recur in the subject for at least 3 months, or at least 6 months, or at least 12 months after the surgical resection of the one or more tumors.
Embodiment 26. A method for inhibiting the recurrence of bladder cancer in a subject who has had one or more bladder tumors surgically resected, the method comprising:

(a) following surgical resection of the one or more bladder tumors, directly injecting an effective amount of a first composition comprising taxane particles into the resection site(s), wherein the taxane particles have a mean particle size (number) of from 0.1 microns to 5 microns;

(b) a first (initial) instilling via intravesical instillation of an effective amount of a second composition comprising a taxane solution or taxane particles having a mean particle size (number) of from 0.1 microns to 5 microns into the bladder of the subject after injecting the first composition; and

(c) instilling via intravesical instillation of an effective amount of the second composition into the bladder of the subject an additional 1-14 times after the first (initial) instilling; wherein the bladder cancer does not recur in the subject for at least 3 months, or at least 6 months, or at least 12 months after the surgical resection of the one or more tumors, thereby inhibiting the recurrence of the bladder cancer.

Embodiment 27. The method of embodiment 26, wherein the instillations are separated by periodic intervals, such as about a week, about 2 weeks, about 3 weeks, about a month, about 2 months, or about 3 months.
Embodiment 28. The method of any one of embodiments 26 or 27, wherein the taxane particles of the first composition have a mean particle size (number) of from 0.1 microns to 1.5 microns, or from 0.4 microns to 1.2 microns, wherein the second composition comprises taxane particles, and wherein the taxane particles of the second composition have a mean particle size (number) of from 0.1 microns to 1.5 microns, or from 0.4 microns to 1.2 microns.
Embodiment 29. The method of any one of embodiments 26 to 28, wherein the taxane particles comprise at least 95% of the taxane.
Embodiment 30. The method of any one of embodiments 26 to 29, wherein the taxane particles of the first composition are docetaxel particles, wherein the second composition comprises taxane particles, and wherein the taxane particles of the second composition are docetaxel particles.
Embodiment 31. The method of embodiment 30, wherein the docetaxel particles, wherein the docetaxel particles have a specific surface area (SSA) of at least 18 m2/g.
Embodiment 32. The method of any one of embodiments 30 or 31, wherein the docetaxel particles have a bulk density (not-tapped) of 0.05 g/cm3 to 0.15 g/cm3.
Embodiment 33. The method of any one of embodiments 26 or 27, wherein the second composition comprises a taxane solution, and wherein the taxane solution is docetaxel solution.
Embodiment 34. The method of any one of embodiments 26 to 33, wherein the first composition and/or the second composition exclude albumin.
Embodiment 35. The method of any one of embodiments 26 to 34, wherein the first composition further comprises a liquid carrier, wherein the first composition comprises a suspension of the taxane particles dispersed in the liquid carrier, wherein the second composition comprises taxane particles, wherein the second composition further comprises a liquid carrier, and wherein the second composition comprises a suspension of the taxane particles dispersed in the liquid carrier.
Embodiment 36. The method of embodiment 35, wherein the liquid carrier is an aqueous carrier.
Embodiment 37. The method of embodiment 26, wherein the aqueous carrier comprises normal saline solution.
Embodiment 38. The method of any one of embodiments 36 or 37, wherein the aqueous carrier comprises a surfactant and/or ethanol.
Embodiment 39. The method of embodiment 38, wherein the aqueous carrier comprises a surfactant, and wherein the surfactant is a polysorbate.
Embodiment 40. The method of embodiment 39, wherein the polysorbate is polysorbate 80, and wherein the polysorbate 80 is present in the liquid carrier at a concentration of about 0.01% w/v to about 1% w/v.
Embodiment 41. The method of any one of embodiments 38 to 40, wherein the aqueous carrier comprises ethanol, and wherein the ethanol is present at a concentration of about 0.1% w/v to about 8% w/v.
Embodiment 42. The method of any one of embodiments 35 to 41, wherein the first composition further comprises a diluent, wherein the carrier and the diluent form a mixture, wherein the first composition is a suspension of the taxane particles dispersed in the carrier/diluent mixture, wherein the second composition comprises taxane particles, wherein the second composition further comprises a diluent, wherein the liquid carrier and the diluent form a mixture, and wherein the second composition is a suspension of the taxane particles dispersed in the liquid carrier/diluent mixture.
Embodiment 43. The method of embodiment 42, wherein the diluent is a normal saline solution.
Embodiment 44. The method of any one of embodiments 30 to 43, wherein the concentration of the docetaxel particles in the first composition is about 1 mg/mL to about 4 mg/mL.
Embodiment 45. The method of any one of embodiment 30 to 44, wherein the second composition comprises docetaxel particles, and wherein the concentration of the docetaxel particles in the second composition is about 1 mg/mL to about 15 mg/mL.
Embodiment 46. The method of any one of embodiments to 26 to 45, wherein the instillation volume of the second composition is about 25 mL.
Embodiment 47. The method of any one of embodiments 26 to 46, wherein the bladder cancer was non-muscle invasive bladder cancer (NMIBC) or muscle invasive bladder cancer (MIBC) prior to the surgical resection of the one or more bladder tumors.
Embodiment 48. A method of treating bladder cancer in a subject, the method comprising:
(a) administering a first administration (first cycle) of an effective amount of a composition comprising taxane particles to a bladder tumor of the subject via intratumoral injection, wherein the taxane particles have a mean particle size (number) of from 0.1 microns to 5 microns,
(b) optionally, administering a second administration (second cycle) of an effective amount of the composition to the bladder tumor via intratumoral injection within a periodic interval following the first administration in (a), and
(c) optionally, administering a third administration (third cycle) of an effective amount of the composition to the bladder tumor via intratumoral injection within a periodic interval following the second administration in (b),
thereby treating the bladder cancer.
Embodiment 49. The method of embodiment 48, further comprising administering one or more additional administrations of the composition to the bladder tumor via intratumoral injection within a periodic interval after each administration.
Embodiment 50. The method of any one of embodiments 48 or 49, wherein the periodic interval is about a week, about 2 weeks, about 3 weeks, about a month, about 2 months, or about 3 months.
Embodiment 51. The method of any one of embodiments 48 to 50, wherein the taxane particles have a mean particle size (number) of from 0.1 microns to 1.5 microns, or from 0.4 microns to 1.2 microns.
Embodiment 52. The method of any one of embodiments 48 to 51, wherein the taxane particles comprise at least 95% of the taxane.
Embodiment 53. The method of any one of embodiments 48 to 52, wherein the taxane particles are docetaxel particles.
Embodiment 54. The method of embodiment 53, wherein the docetaxel particles, wherein the docetaxel particles have a specific surface area (SSA) of at least 18 m2/g.
Embodiment 55. The method of any one of embodiments 53 or 54, wherein the docetaxel particles have a bulk density (not-tapped) of 0.05 g/cm3 to 0.15 g/cm3.
Embodiment 56. The method of any one of embodiments 48 to 55, wherein the composition and/or the taxane particles exclude albumin.
Embodiment 57. The method of any one of embodiments 48 to 56, wherein the composition further comprises a liquid carrier, and wherein the composition comprises a suspension of the taxane particles dispersed in the liquid carrier.
Embodiment 58. The method of embodiment 57, wherein the liquid carrier is an aqueous carrier.
Embodiment 59. The method of embodiment 58, wherein the aqueous carrier comprises normal saline solution.
Embodiment 60. The method of any one of embodiments 58 or 59, wherein the aqueous carrier comprises a surfactant and/or ethanol.
Embodiment 61. The method of embodiment 60, wherein the aqueous carrier comprises a surfactant, and wherein the surfactant is a polysorbate.
Embodiment 62. The method of embodiment 61, wherein the polysorbate is polysorbate 80, and wherein the polysorbate 80 is present in the liquid carrier at a concentration of about 0.01% w/v to about 1% w/v.
Embodiment 63. The method of any one of embodiments 60 to 62, wherein the aqueous carrier comprises ethanol, and wherein the ethanol is present at a concentration of about 0.1% w/v to about 8% w/v.
Embodiment 64. The method of any one of embodiments 57 to 63, wherein the composition further comprises a diluent, wherein the carrier and the diluent form a mixture, and wherein the composition is a suspension of the taxane particles dispersed in the carrier/diluent mixture.
Embodiment 65. The method of embodiment 64, wherein the diluent is a normal saline solution.
Embodiment 66. The method of any one of embodiments 53 to 65, wherein the concentration of the docetaxel particles in the composition is about 1 mg/mL to about 40 mg/mL.
Embodiment 67. The method of any one of embodiments 48 to 66, wherein the bladder cancer is low risk bladder cancer.
Embodiment 68. The method of any one of embodiments 48 to 66, wherein the bladder cancer is intermediate risk or high-risk bladder cancer.
Embodiment 69. A method of administering a tumoricidal dose of a composition comprising taxane particles to a bladder tumor of a subject who has bladder cancer, the method comprising:

(a) administering a first administration (first cycle) of an effective amount of the composition comprising taxane particles to the bladder tumor of the subject via intratumoral injection, wherein the taxane particles have a mean particle size (number) of from 0.1 microns to 5 microns, and

(b) administering a second administration (second cycle) of an effective amount of the composition to the bladder tumor via intratumoral injection within a periodic interval following the first administration in (a), and

(c) optionally, administering a third administration (third cycle) of an effective amount of the composition to the bladder tumor via intratumoral injection within a periodic interval following the second administration in (b),

wherein the bladder tumor is eliminated.
Embodiment 70. The method of any embodiment 69, wherein the periodic interval is about a week, about 2 weeks, about 3 weeks, about a month, about 2 months, or about 3 months.
Embodiment 71. The method of any one of embodiments 69 or 70, wherein the taxane particles have a mean particle size (number) of from 0.1 microns to 1.5 microns, or from 0.4 microns to 1.2 microns.
Embodiment 72. The method of any one of embodiments 69 to 71, wherein the taxane particles comprise at least 95% of the taxane.
Embodiment 73. The method of any one of embodiments 69 to 72, wherein the taxane particles are docetaxel particles.
Embodiment 74. The method of embodiment 73, wherein the docetaxel particles, wherein the docetaxel particles have a specific surface area (SSA) of at least 18 m2/g.
Embodiment 75. The method of any one of embodiments 73 or 74, wherein the docetaxel particles have a bulk density (not-tapped) of 0.05 g/cm3 to 0.15 g/cm3.
Embodiment 76. The method of any one of embodiments 69 to 75, wherein the composition and/or the taxane particles exclude albumin.
Embodiment 77. The method of any one of embodiments 69 to 76, wherein the composition further comprises a liquid carrier, and wherein the composition comprises a suspension of the taxane particles dispersed in the liquid carrier.
Embodiment 78. The method of embodiment 77, wherein the liquid carrier is an aqueous carrier.
Embodiment 79. The method of embodiment 78, wherein the aqueous carrier comprises normal saline solution.
Embodiment 80. The method of any one of embodiments 78 or 79, wherein the aqueous carrier comprises a surfactant and/or ethanol.
Embodiment 81. The method of embodiment 80, wherein the aqueous carrier comprises a surfactant, and wherein the surfactant is a polysorbate.
Embodiment 82. The method of embodiment 81, wherein the polysorbate is polysorbate 80, and wherein the polysorbate 80 is present in the liquid carrier at a concentration of about 0.01% w/v to about 1% w/v.
Embodiment 83. The method of any one of embodiments 80 to 82, the aqueous carrier comprises ethanol, and wherein the ethanol is present at a concentration of about 0.1% w/v to about 8% w/v.
Embodiment 84. The method of any one of embodiments 77 to 83, wherein the composition further comprises a diluent, wherein the carrier and the diluent form a mixture, and wherein the composition is a suspension of the taxane particles dispersed in the carrier/diluent mixture.
Embodiment 85. The method of embodiment 84, wherein the diluent is a normal saline solution.
Embodiment 86. The method of any one of embodiments 73 to 85, wherein the concentration of the docetaxel particles in the composition is about 1 mg/mL to about 40 mg/mL.
Embodiment 87. The method of any one of embodiments 69 to 86, wherein the bladder cancer is low risk bladder cancer.
Embodiment 88. The method of any one of embodiments 69 to 86, wherein the bladder cancer is intermediate risk or high-risk bladder cancer.
Embodiment 89. The method of any one of embodiments 48 to 88, wherein the taxane particles reside at the tumor site after administration of the composition exposing the tumor to the taxane particles for a sustained amount of time sufficient to stimulate the endogenous immune system of the subject resulting in the production of tumoricidal cells and infiltration of the tumoricidal cells in and/or around the tumor site at a level sufficient to treat the tumor.
Embodiment 90. The method of embodiment 89, wherein the stimulation of the endogenous immune system produces a cellular immune response.
Embodiment 91. The method of embodiment 89, wherein the stimulation of the endogenous immune system produces a humoral immune response.
Embodiment 92. The method of any one of embodiments 89 to 91, wherein the sustained amount of time is at least 4 weeks.
Embodiment 93. The method of any one of embodiments 89 to 92, wherein the tumoricidal cells comprise dendritic cells, macrophages, T-cells, B cells, lymphocytes, or natural killer (NK) cells, or combinations thereof.
Embodiment 94. The method of any one of embodiments 1 to 47, wherein the method further comprises directly injecting the first composition into an area outside the resection site margin peripheral to the resection site.

It is contemplated that any embodiment discussed in this specification can be implemented with respect to any method or composition of the disclosure, and vice versa. Furthermore, compositions of the disclosure can be used to achieve methods of the disclosure.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### BRIEF DESCRIPTION OF THE FIGURES

FIG. 1 is a dosing scheme of 9 injection sites for a study of the direct injection of dye into a rabbit bladder wall.

FIG. 2 is a photograph after the 1^{st} injection of dye into the 1^{st} injection site of a rabbit bladder wall.

FIG. 3 is a photograph after the 9^{th} injection of dye into the 9^{th} injection site of a rabbit bladder wall.

FIG. 4 is a graph of mean tumor volumes from Day 17 (Day 1 treatment) to Day 61 post tumor implant in a Human Bladder Cancer (UM-UC-3) Mouse Xenograft Model. Arrows show administration cycle days.

FIG. 5 is a graph of tumor volume of each animal from Day 17 (Day 1 treatment) to Day 61 post tumor implant for Vehicle IT administration (3 cycles) in Human Bladder Cancer (UM-UC-3) Mouse Xenograft Model

FIG. 6 is a graph of tumor volume of each animal from Day 17 (Day 1 treatment) to Day 61 post tumor implant for Docetaxel IV administration (3 cycles) in Human Bladder Cancer (UM-UC-3) Mouse Xenograft Model

FIG. 7 is a graph of tumor volume of each animal from Day 17 (Day 1 treatment) to Day 61 post tumor implant for nanoparticulate docetaxel (nDoce) IT administration (1 cycle) in Human Bladder Cancer (UM-UC-3) Mouse Xenograft Model

FIG. 8 is a graph of tumor volume of each animal from Day 17 (Day 1 treatment) to Day 61 post tumor implant for nDoce IT administration (2 cycles) in Human Bladder Cancer (UM-UC-3) Mouse Xenograft Model

FIG. 9 is a graph of tumor volume of each animal from Day 17 (Day 1 treatment) to Day 61 post tumor implant for nDoce IT administration (3 cycles) in Human Bladder Cancer (UM-UC-3) Mouse Xenograft Model

FIG. 10 is a scatter plot of tumor volumes per animal at Day 1 treatment vs. End of Study in Human Bladder Cancer (UM-UC-3) Mouse Xenograft Model

FIG. 11 is a graph of mean animal body weights from Day 17 (Day 1 treatment) to Day 61 post tumor implant in Human Bladder Cancer (UM-UC-3) Mouse Xenograft Model

FIG. 12 is a graph of mean tumor volumes at Day 61 for each administration group from the bladder cancer xenograft study.

FIG. 13 are photos of animals from each administration group at Day 27, Day 40 and Day 61 post tumor implant from the bladder cancer xenograft study.

FIG. 14 a graph of concentrations of docetaxel in tumor tissue for nDoce 1 cycle, 2 cycles, and 3 cycles from the bladder cancer xenograft study.

FIG. 15 is a photomicrograph of bladder cancer xenograft tissue slide—IT Vehicle Control. H&E. Magnification 2.52×.

FIG. 16 is a photomicrograph of bladder cancer xenograft tissue slide—IT Vehicle Control. H&E. Magnification 6.3×.

FIG. 17 is a photomicrograph of bladder cancer xenograft tissue slide—IT Vehicle Control. H&E. Magnification 25.2×.

FIG. 18 is a photomicrograph of bladder cancer xenograft tissue slide—IV Docetaxel 3 cycles. H&E. Magnification 2.52×.

FIG. 19 is a photomicrograph of bladder cancer xenograft tissue slide—IV Docetaxel 3 cycles. H&E. Magnification 6.3×.

FIG. 20 is a photomicrograph of bladder cancer xenograft tissue slide—IV Docetaxel 3 cycles. H&E. Magnification 25.2×.

FIG. 21 is a photomicrograph of bladder cancer xenograft tissue slide—IT nDoce 2 cycles. H&E. Magnification 2.52×.

FIG. 22 is a photomicrograph of bladder cancer xenograft tissue slide—IT nDoce 2 cycles. H&E. Magnification 6.3×.

FIG. 23 is a photomicrograph of bladder cancer xenograft tissue slide—IT nDoce 3 cycles. H&E. Magnification 2.52×.

FIG. 24 is a photomicrograph of bladder cancer xenograft tissue slide—IT nDoce 3 cycles. H&E. Magnification 2.52×.

FIG. 25 is a photomicrograph of bladder cancer xenograft tissue slide—IT nDoce 3 cycles. H&E. Magnification 25.2×.

FIG. 26 is a photomicrograph of bladder cancer xenograft tissue slide—IT Vehicle Control 3 cycles F4/80 stain. Magnification 2.52×.

FIG. 27 is a photomicrograph of bladder cancer xenograft tissue slide—IV Docetaxel 3 cycles F4/80 stain. Magnification 2.52×.

FIG. 28 is a photomicrograph of bladder cancer xenograft tissue slide—IT nDoce 3 cycles F4/80 stain. Magnification 2.52×.

FIG. 29 are various photomicrographs of Control Cases of bladder cancer xenograft tissue slides. H&E stain and CD68 stain.

FIG. 30 are various photomicrographs of IT nDoce cases of bladder cancer xenograft tissue slides. Top row: One cycle nDoce (1×). Second row: Two cycles of nDoce treatment (2×). Third row: Two cycles of nDoce treatment (2×). Fourth row: Three cycles of nDoce treatment (3×).

FIG. 31 is a graph of the flux of paclitaxel (delivered dose of paclitaxel active drug across a porcine bladder membrane over time) from various paclitaxel formulations.

FIG. 32 is a graph of the flux of paclitaxel (delivered dose of paclitaxel active drug across a porcine intestinal membrane over time) from various paclitaxel formulations.

FIG. 33 is a graph of the flux of docetaxel (delivered dose of docetaxel active drug across a porcine bladder membrane over time) from various docetaxel formulations.

### DETAILED DESCRIPTION

Disclosed herein are compositions and methods for treating bladder cancer, as well as for inhibiting the recurrence of bladder cancer after surgical tumor resections, accomplished by the local administration of compositions of taxane particles having a mean particle size (number) of from 0.1 microns to 5 microns. The taxane particles are solid particles that are not bound to or encapsulated by any other substance. Local administration of the compositions includes direct injection, such as intratumoral injection or direct injection into a tumor resection site, and/or intravesical instillation.

In one aspect disclosed herein is method of treating bladder cancer or inhibiting the recurrence of bladder cancer in a subject, the method comprising: directly injecting an effective amount of a first composition comprising taxane particles into one or more bladder tumor surgical resection sites, wherein the injecting is done following surgical resection of one or more bladder tumors of the subject, wherein the taxane particles have a mean particle size (number) of from 0.1 microns to 5 microns, thereby treating or inhibiting the recurrence of the bladder cancer. In some embodiments, the method still further comprises: instilling via intravesical instillation of an effective amount of the second composition into the bladder of the subject an additional 1 to 14 times after the first (initial) instilling. In some embodiments, the instillations are separated by periodic intervals, such as about a week, or about 2 weeks, or about 3 weeks, or about a month, or about 2 months, or about 3 months.

In another aspect disclosed herein is a method for inhibiting the recurrence of bladder cancer in a subject who has had one or more bladder tumors surgically resected, the method comprising: (a) following surgical resection of the one or more bladder tumors, directly injecting an effective amount of a first composition comprising taxane particles into the resection site(s), wherein the taxane particles have a mean particle size (number) of from 0.1 microns to 5 microns; (b) a first (initial) instilling via intravesical instillation of an effective amount of a second composition comprising a taxane solution or taxane particles having a mean particle size (number) of from 0.1 microns to 5 microns into the bladder of the subject after injecting the first composition; and (c) instilling via intravesical instillation of an effective amount of the second composition into the bladder of the subject an additional 1-14 times after the first (initial) instilling; wherein the bladder cancer does not recur in the subject for at least 3 months, or at least 6 months, or at least 12 months after the after the surgical resection of the one or more tumors, thereby inhibiting the recurrence of the bladder cancer. In some embodiments, the instillations are separated by periodic intervals, such as about a week, or about 2 weeks, or about 3 weeks, or about a month, or about 2 months, or about 3 months.

In another aspect disclosed herein is a method of treating bladder cancer in a subject, the method comprising: (a) administering a first administration (first cycle) of an effective amount of a composition comprising taxane particles to a bladder tumor of the subject via intratumoral injection, wherein the taxane particles have a mean particle size (number) of from 0.1 microns to 5 microns, (b) optionally, administering a second administration (second cycle) of an effective amount of the composition to the bladder tumor via intratumoral injection within a periodic interval following the first administration in (a), and (c) optionally, administering a third administration (third cycle) of an effective amount of the composition to the bladder tumor via intratumoral injection within a periodic interval following the second administration in (b), thereby treating the bladder cancer. In some embodiments, the method further comprises administering one or more additional administrations of the composition to the bladder tumor via intratumoral injection within a periodic interval after each administration. In some embodiments, the periodic interval is about a week, about 2 weeks, about 3 weeks, about a month, about 2 months, or about 3 months.

In still another aspect disclosed herein is a method of administering a tumoricidal dose of a composition comprising taxane particles to a bladder tumor of a subject who has bladder cancer, the method comprising: (a) administering a first administration (first cycle) of an effective amount of the composition comprising taxane particles to the bladder tumor of the subject via intratumoral injection, wherein the taxane particles have a mean particle size (number) of from 0.1 microns to 5 microns, and (b) administering a second administration (second cycle) of an effective amount of the composition to the bladder tumor via intratumoral injection within a periodic interval following the first administration in (a), and (c) optionally, administering a third administration (third cycle) of an effective amount of the composition to the bladder tumor via intratumoral injection within a periodic interval following the second administration in (b), wherein the bladder tumor is eliminated. In some embodiments, the periodic interval is about a week, about 2 weeks, about 3 weeks, about a month, about 2 months, or about 3 months.

Although not bound by theory, it is hypothesized that when a composition comprising taxane particles (including but not limited to paclitaxel particles or docetaxel particles) is administered locally, i.e., directly injected into a bladder tumor (intratumoral injection), and/or directly injected into a resection site of a surgically resected bladder tumor, and/or instilled into the bladder via intravesical instillation, the taxane particles will persist where deposited for a longer time than would solutions of taxanes or albumin coated taxane particles, thus creating a depot effect where the taxane is slowly released from the particles resulting in prolonged local exposure of the surrounding tissues to the taxane. It also is hypothesized that because of the physical characteristics of the taxane particles, the particles when instilled into the bladder via intravesical instillation, will attach onto the inner lining of the bladder and embed within the folds of the inner lining of the bladder resulting in longer residence times and better efficacy than would solutions of taxanes or albumin coated taxane particles. An advantage of the local administration of taxane particles over intravenous (IV) administration of taxane solutions is the avoidance of severe systemic toxicities as seen with IV administration.

Another benefit of the methods disclosed herein is that the exposure of the taxane particles to a bladder cancer tumor after intratumoral administration of the composition for a sustained amount of time is sufficient to stimulate the endogenous immune system resulting in (1) the production of tumoricidal cells, such as dendritic cells, macrophages, T-cells, B cells, lymphocytes, or natural killer (NK) cells, and (2) infiltration of these tumoricidal cells in and/or around the tumor site inducing tumor destruction. In some embodiments, the sustained amount of exposure time is at least 4 weeks. In some embodiments, the sustained amount of exposure time is at least 108, 120, 132, 144, 156, 168, 180, 192, 204, 216, 228, 240, 252, 264, 276, 288, 300, 312, 324, or 336 hours. In various further embodiments, the sustained amount of exposure time is at least 3, 4, 5, 6, 7, or 8 weeks. Without being limited to any specific mechanism, such effect may comprise, for example, providing sufficient time for lymphocytes to activate both their innate as well as adaptive immunological response to the tumor. Without being limited to any specific mechanism, local tumor cell killing by the administration of taxane particles intratumorally into the bladder tumor releases tumor cell antigens which are attached to dendritic cells. The activated dendritic cells may then present tumor-specific antigen to T-cells and other tumoricidal cells that circulate throughout the patient's vascular system as well as enter tissues that contain tumor allowing for destruction of cancer throughout the patient. Thus, methods disclosed herein allow for direct local therapy, as well as indirect immune system-mediated local and systemic cancer cell killing. For example, the methods disclosed herein provide the taxane molecules to act as an adjuvant to stimulate the immune response. Local concentration of taxane remains elevated for greater than 4 days, or at least 14 days, or at least 4 weeks, which provides sufficient time for the tumor to be exposed to the taxane for killing of local tumor cells as well as stimulation of the immune response appropriate for killing of cancer that may be widely disseminated through the body. This stimulation of the immune system by local administration of taxane particles occurs without producing concomitant high levels of taxane in the patient's circulating blood. Thus, local administration of particle taxane does not reduce hematopoiesis in the bone marrow involving reduction in white blood cell numbers such as lymphocytes. Bone marrow suppression is a common side effect of taxanes when given IV due to the high concentrations of circulating taxane. Thus, intratumorally administering the taxane particles is in effect a tumor vaccine given its effect in stimulating the endogenous immune system.

In some embodiments, the stimulation of the endogenous immune systems produces a cellular (cell-mediated) immune response. In other embodiments, the stimulation of the endogenous immune system produces a humoral immune response. In some embodiments, the tumor is treated as a result of the production and tumor infiltration of the tertiary lymphoid structures. In some embodiments, metastases are reduced or eliminated.

Also disclosed herein are methods for stimulating the endogenous immune system of a subject who has a bladder tumor to produce tertiary lymphoid structures (TLSs). Disclosed is a method of producing tertiary lymphoid structures in a subject with a bladder tumor, the method comprising intratumorally administering a composition comprising taxane particles to the tumor of the subject, wherein the taxane particles reside at the tumor site after administration of the composition exposing the tumor to the taxane particles for a sustained amount of time sufficient to stimulate the endogenous immune system of the subject resulting in the production of tertiary lymphoid structures, and infiltration of the tertiary lymphoid structures in and/or around the tumor site. The stimulation of the endogenous immune systems can produce a cellular (cell-mediated) immune response or a humoral immune response. In some embodiments, metastases are reduced or eliminated. The sustained amount of exposure time can be at least 108, 120, 132, 144, 156, 168, 180, 192, 204, 216, 228, 240, 252, 264, 276, 288, 300, 312, 324, or 336 hours, or can be at least 3, 4, 5, 6, 7, or 8 weeks.

The inventors have surprisingly discovered that the intratumoral injection methods disclosed herein stimulate the endogenous immune system resulting in the production of tertiary lymphoid structures that have infiltrated in and around the tumor site inducing tumor destruction. Secondary lymphoid organs develop as part of a genetically preprogrammed process during embryogenesis and primarily serve to initiate adaptive immune response providing a location for interactions between rare antigen-specific naïve lymphocytes and antigen-presenting cells draining from local tissue. Organogenesis of secondary lymphoid tissues can also be recapitulated in adulthood during de novo lymphoid neogenesis of tertiary lymphoid structures (TLSs) and form in the inflamed tissue afflicted by various pathological conditions, including cancer. Organogenesis of mucosal-associated lymphoid tissue such as bronchial-associated lymphoid tissue is one such example. The term TLS can refer to structures of varying organization, from simple clusters of lymphocytes, to sophisticated, segregated structures highly reminiscent of secondary lymphoid organs. A notable difference between lymph nodes and TLSs is the that where lymph nodes are encapsulated, TLSs represent a congregation of immune and stromal cells confined within an organ or tissue.

As used herein, the term “tumor” with respect to bladder cancer means a malignant mass of an abnormal growth of cells found in or on the bladder. Bladder tumors usually form on the inner lining of the bladder (urothelium or transitional epithelium of the bladder); however, tumors can form from the outside of the bladder wall as a metastasis of another cancer. A bladder cancer tumor may or may not be confined to the inner lining of the bladder. The tumor can be further classified by various ways including, but not limited to, the state of the bladder wall invasion, the clinical stage, the pathological stage, and/or the risk factors. For example, bladder cancer can be categorized as “nonmuscle invasive bladder cancer” (NMIBC) or muscle invasive bladder cancer (MIBC) depending on how far the tumors have invaded into the bladder wall. Using the American Joint Committee on Cancer (AJCC) “TNM” staging system, NMIBC includes: 1) tumors that are confined to the inner lining layer (urothelium or transitional epithelium of the bladder), which includes noninvasive papillary carcinoma (Ta) and flat carcinoma in situ or CIS (Tis); and 2) tumors that have invaded the submucosa (subepithelial connective tissue) but have not entered the muscle layer, which includes T1. MIBC is more invasive and includes tumors that have invaded the muscle layer (T2), have gone through the muscle layer and into the fatty tissue layer that surrounds it (T3), and have spread beyond the bladder wall (T4).

Bladder cancer can be categorized by the tumor's clinical stage using 0 and the Roman numerals I to IV. Stage 0 is the earliest stage, while stage IV is the most advanced. Below are the definitions as published by the American Cancer Society, https://www.cancer.org/cancer/bladder-cancer/detection-diagnosis-staging/staging.html

Stage 0a (Ta, N0, M0): The cancer is a non-invasive papillary carcinoma (Ta). It has grown toward the hollow center of the bladder but has not grown into the connective tissue or muscle of the bladder wall. It has not spread to nearby lymph nodes (N0) or distant sites (M0).
Stage 0is (Tis, N0, M0): The cancer is a flat, non-invasive carcinoma (Tis), also known as flat carcinoma in situ (CIS). The cancer is growing in the inner lining layer of the bladder only. It has not grown inward toward the hollow part of the bladder, nor has it invaded the connective tissue or muscle of the bladder wall. It has not spread to nearby lymph nodes (N0) or distant sites (M0).
Stage I (T1, N0, M0): The cancer has grown into the layer of connective tissue under the inner lining layer of the bladder but has not reached the layer of muscle in the bladder wall (T1). The cancer has not spread to nearby lymph nodes (N0) or to distant sites (M0).
Stage II (T2a or T2b, N0, M0): The cancer has grown into the thick muscle layer of the bladder wall, but it has not passed completely through the muscle to reach the layer of fatty tissue that surrounds the bladder (T2). The cancer has not spread to nearby lymph nodes (N0) or to distant sites (M0).
Stage III (T3a, T3b, or T4a, N0, M0): The cancer has grown into the layer of fatty tissue that surrounds the bladder (T3a or T3b). It might have spread into the prostate, uterus, or vagina, but it is not growing into the pelvic or abdominal wall (T4a). The cancer has not spread to nearby lymph nodes (N0) or to distant sites (M0).
Stage IV: One of the following applies: T4b, N0, M0: The cancer has grown through the bladder wall and into the pelvic or abdominal wall (T4b). The cancer has not spread to nearby lymph nodes (N0) or to distant sites (M0). OR Any T, N1 to N3, M0: The cancer has spread to nearby lymph nodes (N1-N3) but not to distant sites (M0). OR Any T, any N, M1: The cancer has spread to distant lymph nodes or to sites such as the bones, liver, or lungs (M1).

Bladder cancer has also been classified into risk level groups by Millan-Rodriguez et. al., 2000, as follows:

Low Risk: grade 1 stage Ta disease or a single grade 1 stage T1 tumor;
Intermediate Risk: multiple grade 1 stage T1 tumors, grade 2 stage Ta disease or a single grade 2 stage T1 tumor; and
High Risk: multiple grade 2 stage T1 tumors, grade 3 stages Ta and T1 disease, or any stage disease associated with carcinoma in situ (CIS or Tis).

Low risk bladder cancer can also include Stage 0, Ta—solitary or primary low-grade tumors. Intermediate risk bladder cancer can also include: Stage 0, Ta—no more than 2 primary low-grade tumors and/or recurrence less than 1 year; and tumors greater than 3 cm in diameter and/or recurrence less than 1 year. High risk bladder cancer can also include any Ti, high-grade, and/or CIS tumors.

The compositions and methods described herein can be used to treat any of the bladder cancer categories and classifications described supra.

As used herein, the terms “treat”, “treatment”, “treated”, or “treating” with respect to bladder cancer means accomplishing one or more of the following: (a) reducing tumor size; (b) reducing tumor growth; (c) reducing or limiting development and/or spreading of metastases; (d) reducing or limiting development of one or more side effects of IV chemotherapy treatment; (e) eliminating a tumor; (f) inhibiting, preventing, or reducing the recurrence of a tumor for at least 3 months, at least 6 months, or at least 12 months. Side effects of IV chemotherapy treatment include, but are not limited to anemia, neutropenia, thrombocytopenia, neurologic toxicities, reduction in appetite, constipation, diarrhea, hair loss, fatigue, nausea/vomiting, and pain.

As used herein, the term “intratumoral injection” means that some or all of the composition, such as a suspension, is directly injected into a bladder tumor mass, and can include one or more injections at one or more injection sites in the tumor in a single administration. As will be understood by those of skill in the art, such direct injection may include injection of some portion of the composition on the periphery of the solid tumor (“peritumorally”), and/or in the surrounding bladder wall tissue, such as if the amount of composition or suspension thereof is too large to all be directly injected into the solid tumor mass. In one embodiment, the composition or suspension thereof is injected in its entirety into the bladder tumor mass. In another embodiment, the composition or suspension in a single administration is injected partially into the bladder tumor mass, the periphery of the bladder tumor mass, and/or the bladder wall tissue surrounding the bladder tumor mass.

As used herein, the term “suspension” means a suspension dosage form composition where taxane particles are dispersed (suspended) within a continuous carrier or a continuous carrier/diluent mixture. The taxane particles can be completely dispersed, partially dispersed and partially dissolved, but not completely dissolved in the carrier or carrier/diluent mixture.

The terms “subject” or “patient” as used herein mean a vertebrate animal. In some embodiments, the vertebrate animal can be a mammal. In some embodiments, the mammal can be a primate, including a human.

As used herein, the term “bladder” means urinary bladder.

The term “room temperature” (RT) as used herein, means 15-30° C. or 20-25° C.

The term “surfactant” or “surface active agent” as used herein, means a compound or a material or a substance that exhibits the ability to lower the surface tension of water or to reduce the interfacial tension between two immiscible substances.

As used herein, the singular forms “a”, “an” and “the” include plural referents unless the context clearly dictates otherwise. “And” as used herein is interchangeably used with “or” unless expressly stated otherwise.

The terms “about” or “approximately” as used herein mean+/−five percent (5%) of the recited unit of measure.

For this application, a number value with one or more decimal places can be rounded to the nearest whole number using standard rounding guidelines, i.e. round up if the number being rounded is 5, 6, 7, 8, or 9; and round down if the number being rounded is 0, 1, 2, 3, or 4. For example, 3.7 can be rounded to 4.

Unless the context clearly requires otherwise, throughout the description and the claims, the words “comprise”, “comprising”, and the like are to be construed in an inclusive or open-ended sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application. The compositions and methods for their use can “comprise,” “consist essentially of,” or “consist of” any of the ingredients or steps disclosed throughout the specification. With respect to the phrase “consisting essentially of,” a basic and novel property of the methods of the present disclosure are their ability to treat and/or inhibit the recurrence of bladder cancer by local administrations of compositions of taxane particles into bladder tumors, or into bladder tumor resection sites following surgical resection procedures.

### Taxane Particles

Taxanes are poorly water-soluble compounds generally having a solubility of less than or equal to 10 mg/mL in water at room temperature. Taxanes are widely used as antineoplastic agents and chemotherapy agents. The term “taxanes” as used herein include paclitaxel (I), docetaxel (II), cabazitaxel (III), and any other taxane or taxane derivatives, non-limiting examples of which are taxol B (cephalomannine), taxol C, taxol D, taxol E, taxol F, taxol G, taxadiene, baccatin III, 10-deacetylbaccatin, taxchinin A, brevifoliol, and taxuspine D, and also include pharmaceutically acceptable salts of taxanes.

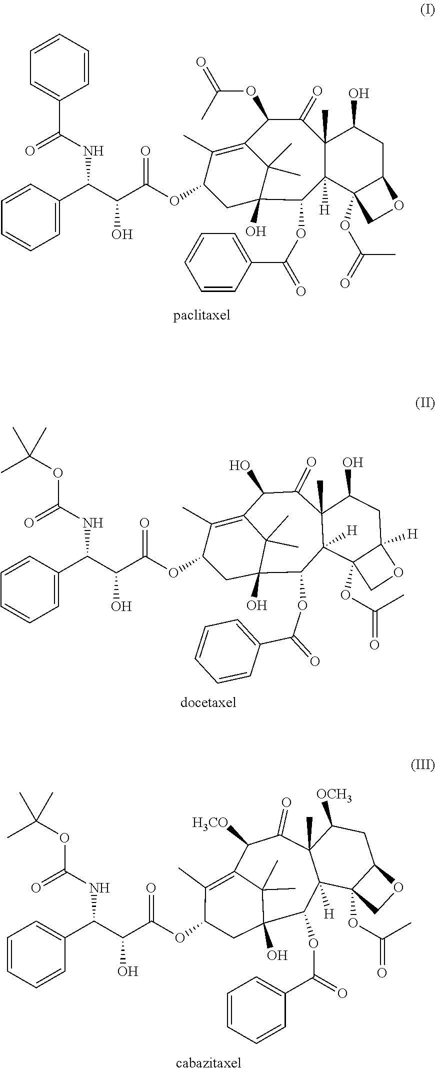

Paclitaxel and docetaxel active pharmaceutical ingredients (APIs) are commercially available from Phyton Biotech LLC, Vancouver, Canada. The docetaxel API contains not less than 90%, or not less than 95%, or not less than 97.5% docetaxel calculated on the anhydrous, solvent-free basis. The paclitaxel API contains not less than 90%, or not less than 95%, or not less than 97% paclitaxel calculated on the anhydrous, solvent-free basis. In some embodiments, the paclitaxel API and docetaxel API are USP and/or EP grade. Paclitaxel API can be prepared from a semisynthetic chemical process or from a natural source such as plant cell fermentation or extraction. Paclitaxel is also sometimes referred to by the trade name TAXOL®, although this is a misnomer because TAXOL® is the trade name of a solution of paclitaxel in polyoxyethylated castor oil and ethanol intended for dilution with a suitable parenteral fluid prior to intravenous infusion. Taxane APIs can be used to make taxane particles. The taxane particles are solid particles. The taxane particles can be paclitaxel particles, docetaxel particles, or cabazitaxel particles, or particles of other taxane derivatives, including particles of pharmaceutically acceptable salts of taxanes.

Taxane particles have a mean particle size (number) of from about 0.1 microns to about 5 microns (about 100 nm to about 5000 nm) in diameter. In some embodiments, the taxane particles have a mean particle size (number) of from about 0.1 microns to about 1.5 microns (about 100 nm to about 1500 nm) in diameter. In some embodiments, the taxane particles have a mean particle size (number) of from about 0.1 microns to less than micron (about 100 nm to less than 1000 nm) in diameter. In preferred embodiments, the taxane particles are solid, uncoated (“neat” or “naked”) individual particles. In some embodiments, the taxane particles are not bound to any substance. In some embodiments, no substances are absorbed or adsorbed onto the surface of the taxane particles. In some embodiments, the taxane or taxane particles are not encapsulated, contained, enclosed or embedded within any substance. In some embodiments, the taxane particles are not coated with any substance. In some embodiments, the taxane particles are not microemulsions, nanoemulsions, microspheres, or liposomes containing a taxane. In some embodiments, the taxane particles are not bound to, encapsulated in, or coated with a monomer, a polymer (or biocompatible polymer), a protein, a surfactant, or albumin. In some embodiments, a monomer, a polymer (or biocompatible polymer), a protein, a surfactant, or albumin is not absorbed or adsorbed onto the surface of the taxane particles. In some embodiments, the taxane particles exclude albumin. In some embodiments, the taxane particles are paclitaxel particles and exclude albumin. In some embodiments, the taxane particles are in crystalline form. In other embodiments, the taxane particles are in amorphous form, or a combination of both crystalline and amorphous form. In some embodiments, the taxane particles of the disclosure contain traces of impurities and byproducts typically found during preparation of the taxane. In some embodiments, the taxane particles comprise at least 90%, at least 95%, at least 96%, at least 97%, at least 98%, at least 99% or 100% of the taxane, meaning the taxane particles consist of or consist essentially of substantially pure taxane.

The taxane particles (including paclitaxel particles, docetaxel particles, or cabazitaxel particles) can have a mean particle size (number) of from 0.1 microns to 5 microns, or from 0.1 microns to 2 microns, or from 0.1 microns to 1.5 microns, or from 0.1 microns to 1.2 microns, or from 0.1 microns to 1 micron, or from 0.1 microns to less than 1 micron, or from 0.1 microns to 0.9 microns, or from 0.1 microns to 0.8 microns, or from 0.1 to 0.7 microns, or from 0.2 microns to 5 microns, or from 0.2 microns to 2 microns, or from 0.2 microns to 1.5 microns, or from 0.2 microns to 1.2 microns, or from 0.2 microns to 1 micron, or from 0.2 microns to less than 1 micron, or from 0.2 microns to 0.9 microns, or from 0.2 microns to 0.8 microns, or from 0.2 microns to 0.7 microns, or from 0.3 microns to 5 microns, or from 0.3 microns to 2 microns, or from 0.3 microns to 1.5 microns, or from 0.3 microns to 1.2 microns, or from 0.3 microns to 1 micron, or from 0.3 microns to less than 1 micron, or from 0.3 microns to 0.9 microns, or from 0.3 microns to 0.8 microns, or from 0.3 microns to 0.7 microns, or from 0.4 microns to 5 microns, or from 0.4 microns to 2 microns, or from 0.4 microns to 1.5 microns, or from 0.4 microns to 1.2 microns, or from 0.4 microns to 1 micron, or from 0.4 microns to less than 1 micron, or from 0.4 microns to 0.9 microns, or from 0.4 microns to 0.8 microns, or from 0.4 microns to 0.7 microns, or from 0.5 microns to 5 microns, or from 0.5 microns to 2 microns, or from 0.5 microns to 1.5 microns, or from 0.5 microns to 1.2 microns, or from 0.5 microns to 1 micron, or from 0.5 microns to less than 1 micron, or from 0.5 microns to 0.9 microns, or from 0.5 microns to 0.8 microns, or from 0.5 microns to 0.7 microns, or from 0.6 microns to 5 microns, or from 0.6 microns to 2 microns, or from 0.6 microns to 1.5 microns, or from 0.6 microns to 1.2 microns, or from 0.6 microns to 1 micron, or from 0.6 microns to less than 1 micron, or from 0.6 microns to 0.9 microns, or from 0.6 microns to 0.8 microns, or from 0.6 microns to 0.7 microns.

The particle size of the taxane particles can be determined by a particle size analyzer instrument and the measurement is expressed as the mean diameter based on a number distribution (number). A suitable particle size analyzer instrument is one which employs the analytical technique of light obscuration, also referred to as photozone or single particle optical sensing (SPOS). A suitable light obscuration particle size analyzer instrument is the ACCUSIZER, such as the ACCUSIZER 780 SIS, available from Particle Sizing Systems, Port Richey, Fla. Another suitable particle size analyzer instrument is one which employs laser diffraction, such as the Shimadzu SALD-7101.

Taxane particles can be manufactured using various particle size-reduction methods and equipment known in the art. Such methods include, but are not limited to conventional particle size-reduction methods such as wet or dry milling, micronizing, disintegrating, and pulverizing. Other methods include “precipitation with compressed anti-solvents” (PCA) such as with supercritical carbon dioxide. In various embodiments, the taxane particles are made by PCA methods as disclosed in US patents U.S. Pat. Nos. 5,874,029, 5,833,891, 6,113,795, 7,744,923, 8,778,181, 9,233,348, 9,814,685; US publications US 2015/0375153, US 2016/0374953; and international patent application publications WO 2016/197091, WO 2016/197100, and WO 2016/197101; all of which are herein incorporated by reference.

In PCA particle size reduction methods using supercritical carbon dioxide, supercritical carbon dioxide (anti-solvent) and solvent, e.g. acetone or ethanol, are employed to generate uncoated taxane particles as small as 0.1 to 5 microns within a well-characterized particle-size distribution. The carbon dioxide and solvent are removed during processing (up to 0.5% residual solvent may remain), leaving taxane particles as a powder. Stability studies show that the paclitaxel particle powder is stable in a vial dose form when stored at room temperature for up to 59 months and under accelerated conditions (40° C./75% relative humidity) for up to six months.

Taxane particles produced by various supercritical carbon dioxide particle size reduction methods can have unique physical characteristics as compared to taxane particles produced by conventional particle size reduction methods using physical impacting or grinding, e.g., wet or dry milling, micronizing, disintegrating, comminuting, microfluidizing, or pulverizing. As disclosed in U.S. Pat. No. 9,233,348, herein incorporated by reference, such unique characteristics include a bulk density (not tapped) between 0.05 g/cm³ and 0.15 g/cm³ and a specific surface area (SSA) of at least 18 m²/g of taxane (e.g., paclitaxel and docetaxel) particles, which are produced by the supercritical carbon dioxide particle size reduction methods described in U.S. Pat. No. 9,814,685 and as described below. This bulk density range is generally lower than the bulk density of taxane particles produced by conventional means, and the SSA is generally higher than the SSA of taxane particles produced by conventional means. These unique characteristics result in significant increases in dissolution rates in water/methanol media as compared to taxanes produced by conventional means. As used herein, the “specific surface area” (SSA) is the total surface area of the taxane particle per unit of taxane mass as measured by the Brunauer-Emmett-Teller (“BET”) isotherm by the following method: a known mass between 200 and 300 mg of the analyte is added to a 30 mL sample tube. The loaded tube is then mounted to a Porous Materials Inc. SORPTOMETER®, model BET-202A. The automated test is then carried out using the BETWIN® software package and the surface area of each sample is subsequently calculated. As will be understood by those of skill in the art, the “taxane particles” can include both agglomerated taxane particles and non-agglomerated taxane particles; since the SSA is determined on a per gram basis it takes into account both the larger agglomerated and smaller non-agglomerated taxane particles in the composition. The agglomerated taxane particles are defined herein as individual taxane particles that are formed by the agglomeration of smaller particles which fuse together forming the larger individual taxane particles, all of which occurs during the processing of the taxane particles. The BET specific surface area test procedure is a compendial method included in both the United States Pharmaceopeia and the European Pharmaceopeia. The bulk density measurement can be conducted by pouring the taxane particles into a graduated cylinder without tapping at room temperature, measuring the mass and volume, and calculating the bulk density.

As disclosed in U.S. Pat. No. 9,814,685, studies showed a SSA of 15.0 m²/g and a bulk density of 0.31 g/cm³ for paclitaxel particles produced by milling paclitaxel in a Deco-PBM-V-0.41 ball mill using a 5 mm ball size, at 600 RPM for 60 minutes at room temperature. Also disclosed in U.S. Pat. No. 9,814,685, one lot of paclitaxel particles had a SSA of 37.7 m²/g and a bulk density of 0.085 g/cm³ when produced by a supercritical carbon dioxide method using the following method: a solution of 65 mg/mL of paclitaxel was prepared in acetone. A BETE MicroWhirl® fog nozzle (BETE Fog Nozzle, Inc.) and a sonic probe (Qsonica, model number Q700) were positioned in the crystallization chamber approximately 8 mm apart. A stainless steel mesh filter with approximately 100 nm holes was attached to the crystallization chamber to collect the precipitated paclitaxel particles. The supercritical carbon dioxide was placed in the crystallization chamber of the manufacturing equipment and brought to approximately 1200 psi at about 38° C. and a flow rate of 24 kg/hour. The sonic probe was adjusted to 60% of total output power at a frequency of 20 kHz. The acetone solution containing the paclitaxel was pumped through the nozzle at a flow rate of 4.5 mL/minute for approximately 36 hours. Additional lots of paclitaxel particles produced by the supercritical carbon dioxide method described above had SSA values of: 22.27 m²/g, 23.90 m²/g, 26.19 m²/g, 30.02 m²/g, 31.16 m²/g, 31.70 m²/g, 32.59 m²/g, 33.82 m²/g, 35.90 m²/g, 38.22 m²/g, and 38.52 m²/g.

As disclosed in U.S. Pat. No. 9,814,685, studies showed a SSA of 15.2 m²/g and a bulk density of 0.44 g/cm³ for docetaxel particles produced by milling docetaxel in a Deco-PBM-V-0.41 ball mill using a 5 mm ball size, at 600 RPM for 60 minutes at room temperature. Also disclosed in U.S. Pat. No. 9,814,685, docetaxel particles had a SSA of 44.2 m²/g and a bulk density of 0.079 g/cm³ when produced by a supercritical carbon dioxide method using the following method: A solution of 79.32 mg/mL of docetaxel was prepared in ethanol. The nozzle and a sonic probe were positioned in the pressurizable chamber approximately 9 mm apart. A stainless steel mesh filter with approximately 100 nm holes was attached to the pressurizable chamber to collect the precipitated docetaxel particles. The supercritical carbon dioxide was placed in the pressurizable chamber of the manufacturing equipment and brought to approximately 1200 psi at about 38° C. and a flow rate of 68 slpm. The sonic probe was adjusted to 60% of total output power at a frequency of 20 kHz. The ethanol solution containing the docetaxel was pumped through the nozzle at a flow rate of 2 mL/minute for approximately 95 minutes). The precipitated docetaxel agglomerated particles and smaller docetaxel particles were then collected from the supercritical carbon dioxide as the mixture is pumped through the stainless steel mesh filter. The filter containing the particles of docetaxel was opened and the resulting product was collected from the filter.

As disclosed in U.S. Pat. No. 9,814,685, dissolution studies showed an increased dissolution rate in methanol/water media of paclitaxel and docetaxel particles made by the supercritical carbon dioxide methods described in U.S. Pat. No. 9,814,685 as compared to paclitaxel and docetaxel particles made by milling paclitaxel and docetaxel using a Deco-PBM-V-0.41 ball mill using a 5 mm ball size, at 600 RPM for 60 minutes at room temperature. The procedures used to determine the dissolution rates are as follows. For paclitaxel, approximately 50 mg of material were coated on approximately 1.5 grams of 1 mm glass beads by tumbling the material and beads in a vial for approximately 1 hour. Beads were transferred to a stainless steel mesh container and placed in the dissolution bath containing methanol/water 50/50 (v/v) media at 37° C., pH 7, and a USP Apparatus II (Paddle), operating at 75 rpm. At 10, 20, 30, 60, and 90 minutes, a 5 mL aliquot was removed, filtered through a 0.22 μm filter and analyzed on a UV/VIS spectrophotometer at 227 nm. Absorbance values of the samples were compared to those of standard solutions prepared in dissolution media to determine the amount of material dissolved. For docetaxel, approximately 50 mg of material was placed directly in the dissolution bath containing methanol/water 15/85 (v/v) media at 37° C., pH 7, and a USP Apparatus II (Paddle), operating at 75 rpm. At 5, 15, 30, 60, 120 and 225 minutes, a 5 mL aliquot was removed, filtered through a 0.22 μm filter, and analyzed on a UV/VIS spectrophotometer at 232 nm. Absorbance values of the samples were compared to those of standard solutions prepared in dissolution media to determine the amount of material dissolved. For paclitaxel, the dissolution rate was 47% dissolved in 30 minutes for the particles made by the supercritical carbon dioxide method versus 32% dissolved in 30 minutes for the particles made by milling. For docetaxel, the dissolution rate was 27% dissolved in 30 minutes for the particles made by the supercritical carbon dioxide method versus 9% dissolved in 30 minutes for the particles made by milling.

In some embodiments, the taxane particles have a SSA of at least 10, at least 12, at least 14, at least 16, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 m²/g. In one embodiment, the taxane particles have an SSA of between about 10 m²/g and about 50 m²/g. In some embodiments, the taxane particles have a bulk density between about 0.050 g/cm³ and about 0.20 g/cm³.

In further embodiments, the taxane particles have a SSA of:

(a) between 16 m²/g and 31 m²/g or between 32 m²/g and 40 m²/g;

(b) between 16 m²/g and 30 m²/g or between 32 m²/g and 40 m²/g;

(c) between 16 m²/g and 29 m²/g or between 32 m²/g and 40 m²/g;

(d) between 17 m²/g and 31 m²/g or between 32 m²/g and 40 m²/g;

(e) between 17 m²/g and 30 m²/g or between 32 m²/g and 40 m²/g;

(f) between 17 m²/g and 29 m²/g or between 32 m²/g and 40 m²/g;

(g) between 16 m²/g and 31 m²/g or between 33 m²/g and 40 m²/g;

(h) between 16 m²/g and 30 m²/g or between 33 m²/g and 40 m²/g;

(i) between 16 m²/g and 29 m²/g or between 33 m²/g and 40 m²/g;

(j) between 17 m²/g and 31 m²/g or between 33 m²/g and 40 m²/g;

(k) between 17 m²/g and 30 m²/g or between 33 m²/g and 40 m²/g;

(l) between 17 m²/g and 29 m²/g or between 33 m²/g and 40 m²/g;

(m) between 16 m²/g and 31 m²/g, or ≥32 m²/g;

(h) between 17 m²/g and 31 m²/g, or 32 m²/g;

(i) between 16 m²/g and 30 m²/g, or 32 m²/g;

(j) between 17 m²/g and 30 m²/g, or 32 m²/g;

(k) between 16 m²/g and 29 m²/g, or 32 m²/g;

(l) between 17 m²/g and 29 m²/g, or 32 m²/g;

(m) between 16 m²/g and 31 m²/g, or A3 m²/g;

(n) between 17 m²/g and 31 m²/g, or 33 m²/g;

(o) between 16 m²/g and 30 m²/g, or 33 m²/g;

(p) between 17 m²/g and 30 m²/g, or 33 m²/g;

(q) between 16 m²/g and 29 m²/g, or 33 m²/g; or

(r) between 17 m²/g and 29 m²/g, or 33 m²/g.

In some embodiments, the taxane particles are non-agglomerated individual particles and are not clusters of multiple taxane particles that are bound together by interactive forces such as non-covalent interactions, van der Waal forces, hydrophilic or hydrophobic interactions, —electrostatic interactions, Coulombic forces, interactions with a dispersion material, or interactions via functional groups. In some embodiments, the taxane particles are individual taxane particles that are formed by the agglomeration of smaller particles which fuse together forming the larger individual taxane particles, all of which occurs during the processing of the taxane particles.

In some embodiments, the taxane particles are paclitaxel particles and have an SSA of at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 m²/g. In other embodiments, the paclitaxel particles have an SSA of 18 m²/g to 50 m²/g, or 20 m²/g to 50 m²/g, or 22 m²/g to 50 m²/g, or 25 m²/g to 50 m²/g, or 26 m²/g to 50 m²/g, or 30 m²/g to 50 m²/g, or 35 m²/g to 50 m²/g, or 18 m²/g to 45 m²/g, or 20 m²/g to 45 m²/g, or 22 m²/g to 45 m²/g, or 25 m²/g to 45 m²/g, or 26 m²/g to 45 m²/g or 30 m²/g to 45 m²/g, or 35 m²/g to 45 m²/g, or 18 m²/g to 40 m²/g, or 20 m²/g to 40 m²/g, or 22 m²/g to 40 m²/g, or 25 m²/g to 40 m²/g, or 26 m²/g to 40 m²/g, or 30 m²/g to 40 m²/g, or 35 m²/g to 40 m²/g.

In some embodiments, the paclitaxel particles have a bulk density (not-tapped) of 0.05 g/cm³ to 0.15 g/cm³, or 0.05 g/cm³ to 0.20 g/cm³.

In some embodiments, the paclitaxel particles have a dissolution rate of at least 40% w/w dissolved in 30 minutes or less in a solution of 50% methanol/50% water (v/v) in a USP II paddle apparatus operating at 75 RPM, at 37° C., and at a pH of 7.

In some embodiments, the taxane particles are docetaxel particles and have an SSA of at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, or at least 42 m²/g. In other embodiments, the docetaxel particles have an SSA of 18 m²/g to 60 m²/g, or 22 m²/g to 60 m²/g, or 25 m²/g to 60 m²/g, or 30 m²/g to 60 m²/g, or 40 m²/g to 60 m²/g, or 18 m²/g to 50 m²/g, or 22 m²/g to 50 m²/g, or 25 m²/g to 50 m²/g, or 26 m²/g to 50 m²/g, or 30 m²/g to 50 m²/g, or 35 m²/g to 50 m²/g, or 40 m²/g to 50 m²/g.

In some embodiments, the docetaxel particles have a bulk density (not-tapped) of 0.05 g/cm³ to 0.15 g/cm³.

In some embodiments, the docetaxel particles have a dissolution rate of at least 20% w/w dissolved in 30 minutes or less in a solution of 15% methanol/85% water (v/v) in a USP II paddle apparatus operating at 75 RPM, at 37° C., and at a pH of 7.

The taxane particles can be packaged into any suitable container such as glass or plastic vials. A non-limiting example of a suitable container is a Type 1, USP, clear-glass vial closed with a bromobutyl rubber stopper and aluminum crimp seal. The taxane particles can be sterilized after the particles are in the container using sterilization methods known in the art such as gamma irradiation or autoclaving.

### Compositions

The compositions of the disclosure comprise taxane particles and are useful for treating bladder cancer and/or for inhibiting the recurrence of bladder cancer following surgical bladder tumor resection procedures, by direct injection of the compositions, e.g., intratumoral injection or direct injection into a tumor resection site; and/or by intravesical instillation of the compositions. The compositions can further comprise a carrier. The carrier can be a liquid (fluid) carrier, such as an aqueous carrier. Non-limiting examples of suitable aqueous carriers include water, such as Sterile Water for Injection USP; normal saline solution (0.9% sodium chloride solution), such as 0.9% Sodium Chloride for Injection USP; dextrose solution, such as 5% Dextrose for Injection USP; and Lactated Ringer's Solution for Injection USP. Non-aqueous based liquid carriers and other aqueous-based liquid carriers can be used. The carrier can be a pharmaceutically acceptable carrier, i.e., suitable for administration to a subject by injection or other routes of administration. The carrier can be any other type of liquid such as emulsions or flowable semi-solids. Non-limiting examples of flowable semisolids include gels and thermosetting gels. The composition can be a suspension, i.e., a suspension dosage form composition where the taxane particles are dispersed (suspended) within a continuous carrier/and or diluent. The taxane particles can be completely dispersed, partially dispersed and partially dissolved, but not completely dissolved in the carrier. In some embodiments, the composition is a suspension of taxane particles dispersed within a continuous carrier. In a preferred embodiment, the carrier is a pharmaceutically acceptable carrier. In preferred embodiments, the composition is sterile. In various embodiments, the composition comprises, consists essentially of, or consists of taxane particles and a liquid carrier, wherein the composition is a suspension of the taxane particles dispersed within the liquid carrier. In some embodiments, the composition consists essentially of or consists of taxane particles and a carrier, wherein the carrier is an aqueous carrier and wherein the composition is a suspension.

The composition of taxane particles and a carrier can be administered as-is. Optionally, the composition of taxane particles and a carrier can further comprise a suitable diluent to dilute the composition in order to achieve a desired concentration (dose) of taxane particles. In some embodiments, the carrier can serve as the diluent; stated another way, the amount of carrier in the composition provides the desired concentration of taxane particles in the composition and no further dilution is needed. A suitable diluent can be a fluid, such as an aqueous fluid. Non-limiting examples of suitable aqueous diluents include water, such as Sterile Water for Injection USP; normal saline solution (0.9% sodium chloride solution), such as 0.9% Sodium Chloride for Injection USP; dextrose solution, such as 5% Dextrose for Injection USP; and Lactated Ringer's Solution for Injection USP. Other liquid and aqueous-based diluents suitable for administration by injection can be used and can optionally include salts, buffering agents, and/or other excipients. In some embodiments, the diluent is sterile. The composition can be diluted with the diluent at a ratio to provide a desired concentration dosage of the taxane particles. For example, the volume ratio of composition to diluent might be in the range of 1:1-1:100 v/v or other suitable ratios. In some embodiments, the composition comprises taxane particles, a carrier, and a diluent, wherein the carrier and diluent form a mixture, and wherein the composition is a suspension of taxane particles dispersed in the carrier/diluent mixture. In some embodiments, the carrier/diluent mixture is a continuous phase and the taxane particles are a dispersed phase.

The composition, carrier, and/or diluent can further comprise functional ingredients such as buffers, salts, osmotic agents, surfactants, viscosity modifiers, rheology modifiers, suspending agents, pH adjusting agents such as alkalinizing agents or acidifying agents, tonicity adjusting agents, preservatives, antimicrobial agents including quaternary ammonium compounds such as benzalkonium chloride and benzethonium chloride, demulcents, antioxidants, antifoaming agents, alcohols such as ethanol, chelating agents, and/or colorants. For example, the composition can comprise taxane particles and a carrier comprising water, a salt, a surfactant, and optionally a buffer. In one embodiment, the carrier is an aqueous carrier and comprises a surfactant, wherein the concentration of the surfactant is 1% or less on a w/w or w/v basis; in other embodiments, the surfactant is less than 0.5%, less than 0.25%, less than 0.1%, or about 0.1%. In other embodiments, the aqueous carrier excludes the surfactants GELUCIRE® (polyethylene glycol glycerides composed of mono-, di- and triglycerides and mono- and diesters of polyethylene glycol) and/or CREMOPHOR® (polyethoxylated castor oil). In some embodiments, the composition or carrier excludes polymers, proteins (such as albumin), polyethoxylated castor oil, and/or polyethylene glycol glycerides composed of mono-, di- and triglycerides and mono- and diesters of polyethylene glycol.

The composition, carrier, and/or diluent can comprise one or more surfactants. Suitable surfactants include by way of example and without limitation polysorbates, lauryl sulfates, acetylated monoglycerides, diacetylated monoglycerides, and poloxamers, such as poloxamer 407. Polysorbates are polyoxyethylene sorbitan fatty acid esters which are a series of partial fatty acid esters of sorbitol and its anhydrides copolymerized with approximately 20, 5, or 4 moles of ethylene oxide for each mole of sorbitol and its anhydrides. Non-limiting examples of polysorbates are polysorbate 20, polysorbate 21, polysorbate 40, polysorbate 60, polysorbate 61, polysorbate 65, polysorbate 80, polysorbate 81, polysorbate 85, and polysorbate 120. Polysorbates containing approximately 20 moles of ethylene oxide are hydrophilic nonionic surfactants. Examples of polysorbates containing approximately 20 moles of ethylene oxide include polysorbate 20, polysorbate 40, polysorbate 60, polysorbate 65, polysorbate 80, polysorbate 85, and polysorbate 120. Polysorbates are available commercially from Croda under the tradename TWEEN™. The number designation of the polysorbate corresponds to the number designation of the TWEEN, e.g., polysorbate 20 is TWEEN 20, polysorbate 40 is TWEEN 40, polysorbate 60 is TWEEN 60, polysorbate 80 is TWEEN 80, etc. USP/NF grades of polysorbate include polysorbate 20 NF, polysorbate 40 NF, polysorbate 60 NF, and polysorbate 80 NF. Polysorbates are also available in PhEur grades (European Pharmacopoeia), BP grades, and JP grades. The term “polysorbate” is a non-proprietary name. The chemical name of polysorbate 20 is polyoxyethylene 20 sorbitan monolaurate. The chemical name of polysorbate 40 is polyoxyethylene 20 sorbitan monopalmitate. The chemical name of polysorbate 60 is polyoxyethylene 20 sorbitan monostearate. The chemical name of polysorbate 80 is polyoxyethylene 20 sorbitan monooleate. In some embodiments, the composition, carrier, and/or diluent can comprise mixtures of polysorbates. In some embodiments, the composition, carrier, and/or diluent comprises polysorbate 20, polysorbate 40, polysorbate 60, polysorbate 65, polysorbate 80, polysorbate 85, and/or polysorbate 120. In some embodiments, the composition, carrier, and/or diluent comprises polysorbate 20, polysorbate 40, polysorbate 60, and/or polysorbate 80. In one embodiment, the composition, carrier, and/or diluent comprises polysorbate 80.

In some embodiments, the composition, carrier, and/or diluent can comprise an alcohol, such as ethanol. The ethanol can be USP grade such as Alcohol USP or Dehydrated Alcohol (200 proof) USP. In some embodiments, the composition comprises taxane particles, a carrier, and optionally a diluent, wherein the carrier and/or diluent comprises water, ethanol, and a polysorbate. In some embodiments, the ethanol is present in the composition, carrier, and/or diluent at a concentration of about 0.1% w/v to about 10% w/v, or about 0.1% w/v to about 8% w/v, or about 2% w/v to about 8% w/v, or about 5% w/v to about 10% w/v, or about 8% w/v. In some embodiments, the ethanol is present in the composition at a concentration of about 0.1 w/v to about 4% w/v, or about 2% w/v to about 4% w/v, or about 3.2% w/v. In one embodiment, the composition is a suspension and the polysorbate is polysorbate 80. In other embodiments, the polysorbate or polysorbate 80 is present in the composition, carrier, and/or diluent at a concentration of between about 0.01% w/v and about 1.5% w/v. The inventors have surprisingly discovered that the recited very small amounts of polysorbate 80 reduce the surface tension at the interface of the taxane particles and the aqueous carrier (such as normal saline solution). These embodiments are typically formulated near the time of use of the composition. In some embodiments, the particles may be coated with the polysorbate or polysorbate 80. In other embodiments, the particles are not coated with the polysorbate or polysorbate 80. In various other embodiments, the polysorbate or polysorbate 80 is present in the composition, carrier, and/or diluent at a concentration of between: about 0.01% w/v and about 1% w/v, about 0.01% w/v and about 0.5% w/v, about 0.01% w/v and about 0.4% w/v, about 0.01% w/v and about 0.35% w/v, about 0.01% w/v and about 0.3% w/v, about 0.01% w/v and about 0.25% w/v, about 0.01% w/v and about 0.2% w/v, about 0.01% w/v and about 0.15% w/v, about 0.01% w/v and about 0.1% w/v, 0.02% w/v and about 1% w/v, about 0.02% w/v and about 0.5% w/v, about 0.02% w/v and about 0.4% w/v, about 0.02% w/v and about 0.35% w/v, about 0.02% w/v and about 0.3% w/v, about 0.02% w/v and about 0.25% w/v, about 0.02% w/v and about 0.2% w/v, about 0.02% w/v and about 0.15% w/v, about 0.02% w/v and about 0.1% w/v, about 0.05% w/v and about 1% w/v, about 0.05% w/v and about 0.5% w/v, about 0.05% w/v and about 0.4% w/v, about 0.05% w/v and about 0.35% w/v, about 0.05% w/v and about 0.3% w/v, about 0.05% w/v and about 0.25% w/v, about 0.05% w/v and about 0.2% w/v, about 0.05% w/v and about 0.15% w/v, about 0.05% w/v and about 0.1% w/v, about 0.1% w/v and about 1% w/v, about 0.1% w/v and about 0.5% w/v, about 0.1% w/v and about 0.4% w/v, about 0.1% w/v and about 0.35% w/v, about 0.1% w/v and about 0.3% w/v, about 0.1% w/v and about 0.25% w/v, about 0.1% w/v and about 0.2% w/v, about 0.1% w/v and about 0.15% w/v, about 0.2% w/v and about 1% w/v, about 0.2% w/v and about 0.5% w/v, about 0.2% w/v and about 0.4% w/v, about 0.2% w/v and about 0.35% w/v, about 0.2% w/v and about 0.3% w/v, about 0.2% w/v and about 0.25% w/v, about 0.3% w/v and about 1% w/v, about 0.3% w/v and about 0.5% w/v, about 0.3% w/v and about 0.4% w/v, or about 0.3% w/v and about 0.35% w/v; or about 0.01%, about 0.05%, about 0.1% w/v, about 0.15% w/v, about 0.16% w/v, about 0.2% w/v, about 0.25% w/v, about 0.3% w/v, about 0.35% w/v, about 0.4% w/v, about 0.45% w/v, about 0.5% w/v, or about 1% w/v.

The composition, carrier, and/or diluent can comprise one or more tonicity adjusting agents. Suitable tonicity adjusting agents include by way of example and without limitation, one or more inorganic salts, electrolytes, sodium chloride, potassium chloride, sodium phosphate, potassium phosphate, sodium, potassium sulfates, sodium and potassium bicarbonates and alkaline earth metal salts, such as alkaline earth metal inorganic salts, e.g., calcium salts, and magnesium salts, mannitol, dextrose, glycerin, propylene glycol, and mixtures thereof.

The composition, carrier, and/or diluent can comprise one or more buffering agents. Suitable buffering agents include by way of example and without limitation, dibasic sodium phosphate, monobasic sodium phosphate, citric acid, sodium citrate, tris(hydroxymethyl)aminomethane, bis(2-hydroxyethyl)iminotris-(hydroxymethyl)methane, and sodium hydrogen carbonate and others known to those of ordinary skill in the art. Buffers are commonly used to adjust the pH to a desirable range for intratumoral or intravesical use.

The composition, carrier, and/or diluent can comprise one or more demulcents. A demulcent is an agent that forms a soothing film over a mucous membrane, such as the membranes lining the peritoneum and organs therein. A demulcent may relieve minor pain and inflammation and is sometimes referred to as a mucoprotective agent. Suitable demulcents include cellulose derivatives ranging from about 0.2 to about 2.5% such as carboxymethylcellulose sodium, hydroxyethyl cellulose, hydroxypropyl methylcellulose, and methylcellulose; gelatin at about 0.01%; polyols in about 0.05 to about 1%, also including about 0.05 to about 1%, such as glycerin, polyethylene glycol 300, polyethylene glycol 400, and propylene glycol; polyvinyl alcohol from about 0.1 to about 4%; povidone from about 0.1 to about 2%; and dextran 70 from about 0.1% when used with another polymeric demulcent described herein.

The composition, carrier, and/or diluent can comprise one or more alkalinizing agents to adjust the pH. As used herein, the term “alkalizing agent” is intended to mean a compound used to provide an alkaline medium. Such compounds include, by way of example and without limitation, ammonia solution, ammonium carbonate, potassium hydroxide, sodium carbonate, sodium bicarbonate, and sodium hydroxide and others known to those of ordinary skill in the art

The composition, carrier, and/or diluent can comprise one or more acidifying agents to adjust the pH. As used herein, the term “acidifying agent” is intended to mean a compound used to provide an acidic medium. Such compounds include, by way of example and without limitation, acetic acid, amino acid, citric acid, nitric acid, fumaric acid and other alpha hydroxy acids, hydrochloric acid, ascorbic acid, and nitric acid and others known to those of ordinary skill in the art.

The composition, carrier, and/or diluent can comprise one or more antifoaming agents. As used herein, the term “antifoaming agent” is intended to mean a compound or compounds that prevents or reduces the amount of foaming that forms on the surface of the fill composition. Suitable antifoaming agents include by way of example and without limitation, dimethicone, SIMETHICONE, octoxynol and others known to those of ordinary skill in the art.

The composition, carrier, and/or diluent can comprise one or more viscosity modifiers that increase or decrease the viscosity of the suspension. Suitable viscosity modifiers include methylcellulose, hydroxypropyl methycellulose, mannitol, polyvinylpyrrolidone, cross-linked acrylic acid polymers such as carbomer, and others known to those of ordinary skill in the art. The composition, carrier, and/or diluent can further comprise rheology modifiers to modify the flow characteristics of the composition to allow it to adequately flow through devices such as injection needles or tubes. Non-limiting examples of viscosity and rheology modifiers can be found in “Rheology Modifiers Handbook—Practical Use and Application” Braun, William Andrew Publishing, 2000.

The concentrations of taxane particles in the compositions can be at amounts effective for treatment of bladder cancer by direct injection and/or intravesical instillation of the compositions. In one embodiment, the concentration of the taxane particles in the composition is between about 0.1 mg/mL and about 100 mg/mL. In various further embodiments, the concentration of taxane particles in the composition is between: about 0.5 mg/mL and about 100 mg/mL, about 1 mg/mL and about 100 mg/mL, about 2 mg/mL and about 100 mg/mL, about 5 mg/mL and about 100 mg/mL, about 10 mg/mL and about 100 mg/mL, about 25 mg/mL and about 100 mg/mL, about 30 mg/mL and about 100 mg/mL, about 0.1 mg/mL and about 75 mg/mL, about 0.5 mg/mL and about 75 mg/mL, about 1 mg/mL and about 75 mg/mL, about 2 mg/mL and about 75 mg/mL, about 5 mg/mL and about 75 mg/mL, about 10 mg/mL and about 75 mg/mL, about 25 mg/mL and about 75 mg/mL, about 30 mg/mL and about 75 mg/mL, about 0.1 mg/mL and about 50 mg/mL, about 0.5 mg/mL and about 50 mg/mL, about 1 mg/mL and about 50 mg/mL, about 2 mg/mL and about 50 mg/mL, about 5 mg/mL and about 50 mg/mL, about 10 mg/mL and about 50 mg/mL, about 25 mg/mL and about 50 mg/mL, about 30 mg/mL and about 50 mg/mL, about 0.1 mg/mL and about 40 mg/mL, about 0.5 mg/mL and about 40 mg/mL, about 1 mg/mL and about 40 mg/mL, about 2 mg/mL and about 40 mg/mL, about 5 mg/mL and about 40 mg/mL, about 10 mg/mL and about 40 mg/mL, about 25 mg/mL and about 40 mg/mL, about 30 mg/mL and about 40 mg/mL, about 0.1 mg/mL and about 30 mg/mL, about 0.5 mg/mL and about 30 mg/mL, about 1 mg/mL and about 30 mg/mL, about 2 mg/mL and about 30 mg/mL, about 5 mg/mL and about 30 mg/mL, about 10 mg/mL and about 30 mg/mL, about 25 mg/mL and about 30 mg/mL, about 0.1 mg/mL and about 25 mg/mL, about 0.5 mg/mL and about 25 mg/mL, about 1 mg/mL and about 25 mg/mL, about 2 mg/mL and about 25 mg/mL, about 5 mg/mL and about 25 mg/mL, about 10 mg/mL and about 25 mg/mL, about 0.1 mg/mL and about 20 mg/mL, about 0.5 mg/mL and about 20 mg/mL, about 1 mg/mL and about 20 mg/mL, about 2 mg/mL and about 20 mg/mL, about 5 mg/mL and about 20 mg/mL, about 10 mg/mL and about 20 mg/mL, about 0.1 mg/mL and about 15 mg/mL, about 0.5 mg/mL and about 15 mg/mL, about 1 mg/mL and about 15 mg/mL, about 2 mg/mL and about 15 mg/mL, about 5 mg/mL and about 15 mg/mL, about 10 mg/mL and about 15 mg/mL, about 0.1 mg/mL and about 10 mg/mL, about 0.5 mg/mL and about 10 mg/mL, about 1 mg/mL and about 10 mg/mL, about 2 mg/mL and about 10 mg/mL, about 5 mg/mL and about 10 mg/mL, about 0.1 mg/mL and about 5 mg/mL, about 0.5 mg/mL and about 5 mg/mL, about 1 mg/mL and about 5 mg/mL, about 2 mg/mL and about 5 mg/mL, about 0.1 mg/mL and about 2 mg/mL, about 0.5 mg/mL and about 2 mg/mL, about 1 mg/mL and about 2 mg/mL, about 0.1 mg/mL and about 1 mg/mL, about 0.5 mg/mL and about 1 mg/mL, about 0.1 mg/mL and about 0.5 mg/mL, about 3 mg/mL and about 8 mg/mL, or about 4 mg/mL and about 6 mg/mL; or at least about 0.1, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 61, 65, 70, 75, or 100 mg/mL; or about 0.1, 0.5, 1, 2, 3, 4, 5, 6, 7, 8, 9, 10, 11, 12, 13, 14, 15, 16, 17, 18, 19, 20, 25, 30, 35, 40, 41, 42, 43, 44, 45, 46, 47, 48, 49, 50, 55, 60, 61, 65, 70, 75, or 100 mg/mL. The taxane particles may be the sole therapeutic agent administered, or may be administered with other therapeutic agents.

In various embodiments, the composition comprises docetaxel particles, a carrier, and a diluent, wherein the concentration of docetaxel particles in the composition (including the carrier and diluent) is from about 1 mg/mL to about 40 mg/mL, or from about 1 mg/mL to about 30 mg/mL, or from about 1 mg/mL to about 20 mg/mL, or from about 1 mg/mL to about 15 mg/mL, or from about 1 mg/mL to about 10 mg/mL, or from about 1 mg/mL to about 8 mg/mL, or from about 1 mg/mL to about 4 mg/mL, or about 1 mg/mL, or about 2 mg/mL, or about 3 mg/mL, or about 4 mg/mL, or about 5 mg/mL, or about 6 mg/mL, or about 7 mg/mL, or about 8 mg/mL. In further embodiments, the carrier is an aqueous carrier which can be a saline solution, such as normal saline solution and the diluent is an aqueous diluent which can be a saline solution, such as normal saline solution. In further embodiments, the aqueous carrier comprises a polysorbate, such as polysorbate 80, and/or ethanol.

Taxane solutions useful for intravesical instillation, include paclitaxel solutions or docetaxel solutions, and are compositions where the taxane is completely dissolved. For example, a suitable docetaxel solution is TAXOTERE®, which is a commercially available formulation of 20 mg/mL of docetaxel dissolved in a solution of 50% v/v polysorbate 80 and 50% v/v dehydrated alcohol. The taxane solution, e.g. docetaxel solution, can be diluted with a suitable diluent such as those described supra to a desired dose concentration for intravesical instillation, e.g., 0.1 mg/mL to 5 mg/mL.

### Kits

The present disclosure also provides kits, comprising:

(a) a first vial comprising, consisting essentially of, or consisting of taxane particles having a mean particle size (number) of from 0.1 to 5 microns;

(b) a second vial comprising a pharmaceutically acceptable carrier; and

(c) instructions for reconstituting the taxane particles into a suspension useful for intravesical instillation, or for direct injection, such as intratumoral injection or injection into a tumor resection site, by: combining the contents of the first vial and the second vial to form the suspension and optionally diluting the suspension with a diluent.

In some embodiments, the taxane particles are docetaxel particles. The docetaxel particles in the first vial can be in a powder form. The amount of docetaxel particles in the first vial can be at any amount suitable for a desired dose level after reconstituting the particles into a suspension. In one embodiment, the amount of docetaxel particles in the first vial is 100 mg. The docetaxel particles in the first vial can be the sole ingredient in the first vial. In some embodiments, the docetaxel particles have a mean particle size (number) of from 0.1 microns to 1.5 microns. In other embodiments, the docetaxel particles have a mean particle size (number) of from 0.4 microns to 1.2 microns. In some embodiments, the docetaxel particles have a specific surface area (SSA) of at least 18 m²/g; and/or a bulk density (not-tapped) of 0.05 g/cm³ to 0.15 g/cm³. The pharmaceutically acceptable carrier can be an aqueous carrier such as normal saline solution. The carrier can further comprise a surfactant such as a polysorbate. In some embodiments, the polysorbate is polysorbate 80. In some embodiments, the polysorbate or polysorbate 80 is at a concentration of between about 0.01% w/v and about 1% w/v. In some embodiments, the amount of polysorbate 80 in the carrier in the second vial is about 1% w/v. In some embodiments, the carrier can further comprise an alcohol such as ethanol. In some embodiments, the amount of ethanol in the carrier in the second vial is about 8% w/v. The kits can include multiple vials of taxane particles and carrier solutions to allow for large volumes of reconstituted suspension available for instillation. The kit can further comprise a diluent such as normal saline solution. The amount of diluent can be adjusted to provide a desired dose concentration and volume. When the suspension of docetaxel particles and a carrier containing a polysorbate and ethanol is diluted with the diluent, excessive dissolution of the docetaxel particles is prevented.

Any suitable vial can be used in the kits. A non-limiting example of a suitable vial is a Type 1, USP, clear-glass vial closed with a bromobutyl rubber stopper and aluminum crimp seal. The volumes of the vials can vary depending on the amount of taxane particles, the volume of the carrier, and the volume of the final reconstituted suspension. The vials and their contents can be sterilized using sterilization methods known in the art such as gamma irradiation or autoclaving. In some embodiments, the contents of the vials are sterile. The kits can be configured for single-dose or multiple-dose administration.

A non-limiting exemplary procedure for preparing a docetaxel suspension composition from a kit for either direct injection or intravesical instillation is as follows: Vial 1 contents: 100 mg docetaxel particles Vial 2 contents: a carrier containing 1% w/v polysorbate 80 and 8% w/v ethanol dissolved in normal saline solution Diluent: normal saline solution 1. Using a syringe with a suitable gauge needle, add 1 mL of the carrier from the second vial into the first vial containing 100 mg docetaxel particles. 2. Vigorously hand shake the first vial with inversions to make sure all the particles adhering to the interior of the vial and stopper are wetted. 3. Immediately after shaking, use a syringe with a suitable gauge needle to add a suitable volume of the diluent to the first vial to dilute the suspension to a desired dose concentration level and volume, and hand shake the vial for another 1 minute. Periodically examine the suspension for any large visible clumps. If present, continue hand mixing until the suspension is properly dispersed.

5. After mixing, allow the suspension to sit undisturbed for at least 5 minutes to reduce entrapped air and foam.

The suspension can be stored at room temperature and should be administered within 24 hours after reconstitution.

The compositions, suspensions, and kits of the disclosure can include any embodiment or combination of embodiments described herein including any embodiments of the taxane particles, any embodiments of the carriers and diluents, any embodiments of the polysorbate or polysorbate 80 concentrations, and any embodiments of the ethanol concentrations. The compositions, suspensions, and kits can exclude polymers, proteins (such as albumin), polyethoxylated castor oil, and/or polyethylene glycol glycerides composed of mono-, di- and triglycerides and mono- and diesters of polyethylene glycol. The compositions and kits can further comprise other components as appropriate for given taxane particles.

### Methods of Administration/Treatment

The compositions comprising taxane particles described and disclosed supra can be used in methods for the treatment of bladder cancer and for the inhibition of bladder cancer recurrence after tumor resection, by local administration of the compositions including direct injection, such as intratumoral injection or injection into a tumor resection site, and/or by intravesical instillation.

Surgical resection procedures, including but not limited to transurethral resection of bladder tumor (TURBT), are used to remove bladder cancer tumors from the bladder wall of a subject. Surgical tumor resection includes tumor removal and if necessary, partial cystectomy. A TURBT procedure generally employs the use of a cystoscope inserted through the urethra into the bladder through which a tool (usually a wire loop) is used to surgically remove the tumor. TURBT procedures include one-stage and two-stage resection. Surgical resection procedures are known in the art and various tools and techniques are used for resection, non-limiting examples of which include wire loops, lasers, and fulguration (high-energy electricity). However, the bladder cancer often recurs after a surgical tumor resection procedure.

Administering a composition comprising taxane particles by directly injecting it into the resection site after tumor resection can be used as further treatment of the bladder cancer and can help to inhibit the recurrence of the bladder cancer. The tumor resection site is the region where a visible tumor mass and margin (normal tissue on the border of the tumor) have been surgically removed, and can be identified visually. The direct injection of the composition into the resection site can be administered soon after the resection procedure (e.g., less than 2 hours while the subject is still under the effects of the anesthesia) or can be administered at a later time. The method of administering the composition into the resection site includes one or more direct injections into the resection site during a single administration. As a non-limiting example, in one administration, 8 injections spaced approximately 1 cm apart in a grid-like pattern throughout the resection site, including up to 5 mm outside the resection margin, can be given to fully cover the resection site with the composition where each of these injections can be about 0.5 mL each for a total of 4 mL of composition. In some embodiments, the total amount of injected composition is 1 to 5 mL. In some embodiments, the total amount of injected composition is 3 to 5 mL. In some embodiments, the total amount of injection composition is 4 mL. Adjustable tip-length cystoscopy needles can be used for injection into the resected bladder wall. The needle tip can be adjusted to 2 mm for injections in the dome area of the bladder and 3-4 mm for injections in the side area of the bladder. Direct visualization of the needle tip can be aided by use of a cystoscope during the procedure. A 70°-degree diagonal cystoscope view can be used. The injections can be given in the resected tumor margin area, which is part of the resection site. The injections can also be given outside the resection site margin peripheral to the resection site to cover an area of surrounding bladder wall tissue that is not included in the resection site, if desired. In various embodiments, the surrounding bladder wall tissue is up to 2 mm, or up to 5 mm, or up to 10 mm, or up to 15 mm, or up to 20 mm outside the edge of the resection site margin. In some embodiments, the surrounding bladder wall tissue is 1, 2, 3, 4, 5, 6, 7, 8, 9, 10, 11, 12, 13, 14, 15, 16, 17, 18, 19, or 20 mm outside the edge of the resection site margin. The injections can be administered though the urethra. The injections can be administered to one or more resection sites corresponding to the number of tumors resected in the surgical resection procedure.

One embodiment is a method of treating bladder cancer or inhibiting the recurrence of bladder cancer in a subject, the method comprising: directly injecting an effective amount of a composition comprising taxane particles into one or more bladder tumor surgical resection sites, wherein the injecting is done following surgical resection of one or more bladder tumors of the subject, wherein the taxane particles have a mean particle size (number) of from 0.1 microns to 5 microns, thereby treating or inhibiting the recurrence of the bladder cancer. In some embodiments, the bladder cancer is intermediate risk or high-risk bladder cancer. In some embodiments, the bladder cancer is NMIBC. In other embodiments, the bladder cancer is MIBC. In some embodiments, the bladder cancer does not recur in the subject for at least 3 months, or at least 6 months, or at least 12 months after the surgical resection of the one or more bladder tumors. In some embodiments, the taxane particles are docetaxel particles. In some embodiments, the concentration of the docetaxel particles in the composition is about 1 mg/mL to about 4 mg/mL.

Intravesical instillation of a composition comprising taxane particles is another method of the disclosure which can be used to treat bladder cancer. The intravesical instillation of a docetaxel particles suspension will establish a depot of drug within the bladder, providing sustained release of docetaxel within the bladder over time. Intravesical instillation procedures are known in the art. For intravesical instillation, the composition (including carrier and any diluent) should be at suitable volume to supply a sufficient dose volume for intravesical instillation, i.e., where the volume of the dose is sufficient to expose the bladder tissues to the composition. Generally, the volume of the composition (including carrier and any diluent) is less than 100 mL. In some embodiments, the volume of the composition is about 10 mL to about 100 mL, or about 20 mL to about 80 mL, or about 25 mL to about 75 mL, or about 10 mL to about 50 mL, or about 15 mL to about 45 mL, or about 20 mL to about 40 mL, or about 25 mL to about 35 mL, or about 20 mL to about 30 mL, or about 25 mL. Dwell times for intravesical instillation generally can be from 30 minutes to 2 hours. Intravesical instillation of a composition comprising taxane particles can be administered to treat bladder tumors, especially low risk bladder tumors with or without surgical tumor resection. Intravesical instillation of a composition comprising taxane particles can also be used to help inhibit the recurrence of bladder cancer following a surgical tumor resection procedure, or following a surgical tumor resection procedure plus direct injection of a composition comprising taxane particles into the resection site. During a surgical tumor resection procedure, cancer cell from the tumor can release from the tumor and implant elsewhere within the bladder. Intravesical instillation can wash these cancer cells away before they embed in the bladder wall or kill the cells before they have a chance to grow into a tumor mass. Because of the physical characteristics of the taxane particles, the particles when instilled into the bladder via intravesical instillation, can attach onto the inner lining of the bladder and embed within the folds of the inner lining of the bladder resulting in long residence times and creating a depot effect where the taxane is slowly released from the particles. Intravesical instillation of a composition comprising taxane particles can be administered initially within a certain time period, (for example, including but not limited to time periods of about 12 hours, about a day, about a week, or about a month), either following a surgical tumor resection procedure, or following a surgical tumor resection procedure plus direct injection of a composition comprising taxane particles into the resection site. One or more subsequent instillations of the composition can be administered following the initial instillation. The subsequent instillations can be separated by periodic intervals. As non-limiting examples, the periodic intervals can be about a day, about a week, about two weeks, about three weeks, about a month, about two months, or about 3 months. In one non-limiting example, 5 subsequent instillations can be administered on a weekly basis two weeks following the initial instillation, and additionally, 3 more subsequent instillations can be administered weekly 3 months after the 5^{th} subsequent instillation, followed by 3 more subsequent instillations administered weekly 3 months after the 8^{th} subsequent instillation, followed by 3 more subsequent instillations administered weekly 3 months after the 11^{th} subsequent instillation, for a total of 14 subsequent instillations administered after the initial instillation. Devices such as catheters and needles can be used to administer the composition to the bladder. Studies have been conducted showing chemical compatibility and suitability of Foley Catheters and InjeTAK® Needles with docetaxel particle suspensions.

One embodiment is a method of treating bladder cancer or inhibiting the recurrence of bladder cancer in a subject, the method comprising: directly injecting an effective amount of a first composition comprising taxane particles into one or more bladder tumor surgical resection sites, wherein the injecting is done following surgical resection of one or more bladder tumors of the subject, wherein the taxane particles have a mean particle size (number) of from 0.1 microns to 5 microns, thereby treating or inhibiting the recurrence of the bladder cancer. In some embodiments, the injection occurs within 2 hours after the surgical resection procedure. In some embodiments, the method further comprises: a first (initial) instilling via intravesical instillation of an effective amount of a second composition comprising a taxane solution or taxane particles having a mean particle size (number) of from 0.1 microns to 5 microns into the bladder of the subject after injecting the first composition. In some embodiments, the first (initial) instilling occurs within 2 hours after the injection. In some embodiments, the method still further comprises: instilling via intravesical instillation of an effective amount of the second composition into the bladder of the subject an additional 1 to 14 times after the first (initial) instilling. In some embodiments, the additional instilling begins after the surgical resection site has healed. In some embodiments, the instillations are separated by periodic intervals, such as about a week, about 2 weeks, about 3 weeks, about a month, about 2 months, or about 3 months. In some embodiments, the bladder cancer is intermediate risk or high-risk bladder cancer. In some embodiments, the bladder cancer does not recur in the subject for at least 3 months, or at least 6 months, or at least 12 months after the surgical resection of the one or more bladder tumors. In some embodiments, the taxane particles in the first and second compositions are docetaxel particles. In some embodiments, the taxane particles in the first and second composition are paclitaxel particles. In some embodiments, the taxane particles in the first composition are docetaxel particles and the taxane particles in the second composition are paclitaxel particles. In some embodiments, the taxane particle in the first composition are paclitaxel particles and the taxane particles in the second composition are docetaxel particles. In some embodiments, the taxane particles in the first composition are docetaxel particles and the taxane solution in the second composition is docetaxel solution. In some embodiments, the taxane particles in the first composition are docetaxel particles and the taxane solution in the second composition is paclitaxel solution. In some embodiments, the taxane particles in the first composition are paclitaxel particles and the taxane solution in the second composition is docetaxel solution. In some embodiments, the taxane particles in the first composition are paclitaxel particles and the taxane solution in the second composition is paclitaxel solution. In some embodiments, the concentration of the docetaxel particles in the first composition is about 1 mg/mL to about 4 mg/mL. In some embodiments, the concentration of the docetaxel particles in the second composition is about 1 mg/mL to about 15 mg/mL. In some embodiments the taxane solution is docetaxel solution. In some embodiments, the bladder cancer is NMIBC. In other embodiments, the bladder cancer is MIBC.

One embodiment is a method for inhibiting the recurrence of bladder cancer in a subject who has had one or more bladder tumors surgically resected, the method comprising: (a) following surgical resection of the one or more bladder tumors, directly injecting an effective amount of a first composition comprising taxane particles into the resection site(s), wherein the taxane particles have a mean particle size (number) of from 0.1 microns to 5 microns; (b) a first (initial) instilling via intravesical instillation of an effective amount of a second composition comprising a taxane solution or taxane particles having a mean particle size (number) of from 0.1 microns to 5 microns into the bladder of the subject after injecting the first composition; and (c) instilling via intravesical instillation of an effective amount of the second composition into the bladder of the subject an additional 1-14 times after the first (initial) instilling; wherein the bladder cancer does not recur in the subject for at least 3 months, or at least 6 months, or at least 12 months after the after the surgical resection of the one or more tumors, thereby inhibiting the recurrence of the bladder cancer. In some embodiments, the instillations are separated by periodic intervals, such as about a week, about 2 weeks, about 3 weeks, about a month, about 2 months, or about 3 months. In some embodiments, the bladder cancer was intermediate risk or high-risk bladder cancer prior to the surgical resection of the one or more bladder tumors. In some embodiments, the taxane particles are docetaxel particles. In some embodiments, the concentration of the docetaxel particles in the first composition is about 1 mg/mL to about 4 mg/mL. In some embodiments, the concentration of the docetaxel particles in the second composition is about 1 mg/mL to about 15 mg/mL. In some embodiments, the taxane solution is docetaxel solution. In some embodiments, the bladder cancer is NMIBC. In other embodiments, the bladder cancer is MIBC.

Another method of the disclosure useful for the treatment of bladder cancer is the intratumoral injection of a composition of taxane particles for one, two, three, or more administration cycles. Intratumoral injection of taxane particles into solid tumors is disclosed in international patent application publication WO 2017/176628. However, it is now shown that intratumorally injecting a composition comprising docetaxel particles into a bladder cancer tumor in a mouse xenograft model for two or three administration cycles is tumoricidal. As used herein, the term “intratumoral injection” means that some or all of the composition, such as a suspension, is directly injected into a bladder tumor mass. As will be understood by those of skill in the art, such direct injection may include injection of some portion of the composition on the periphery of the solid tumor (“peritumorally”), and/or in the bladder wall tissue surrounding the tumor, such as if the amount of composition or suspension thereof is too large to all be directly injected into the solid tumor mass. In one embodiment, the composition or suspension thereof is injected in its entirety into the bladder tumor mass. As used herein, the terms “cycle” with respect to administration via intratumoral injection of a composition comprising taxane particles into a bladder tumor means a single administration of the composition by intratumoral injection. The two or more administration cycles can be separated by a periodic interval. As non-limiting examples, the periodic intervals can be about a day, about a week, about two weeks, about three weeks, about a month, about two months, or about a quarter. The injections can be administered though the urethra. Intravesical instillations of a composition comprising taxane particles can also be administered in-between or after the two or more administration cycles of the intratumoral injections.

One embodiment is a method of treating bladder cancer in a subject, the method comprising: (a) administering a first administration (first cycle) of an effective amount of a composition comprising taxane particles to a bladder tumor of the subject via intratumoral injection, wherein the taxane particles have a mean particle size (number) of from 0.1 microns to 5 microns, and (b) administering a second administration (second cycle) of an effective amount of the composition to the bladder tumor via intratumoral injection within a periodic interval following the first administration in (a), and (c) optionally, administering a third administration (third cycle) of an effective amount of the composition to the bladder tumor via intratumoral injection within a periodic interval following the second administration in (b), wherein the bladder tumor is eliminated, thereby treating the bladder cancer. In some embodiments, the method further comprises administering one or more additional administrations of the composition to the bladder tumor via intratumoral injection within a periodic interval after each administration. In some embodiments, the periodic interval is about a week. In some embodiments, the taxane particles are docetaxel particles. In some embodiments, the bladder cancer is a low risk bladder cancer. In other embodiments, the bladder cancer is intermediate risk or high-risk bladder cancer. In some embodiments, the bladder cancer is NMIBC. In other embodiments, the bladder cancer is MIBC.

One embodiment is a method of administering a tumoricidal dose of a composition comprising taxane particles to a bladder tumor of a subject who has bladder cancer, the method comprising: (a) administering a first administration (first cycle) of an effective amount of the composition comprising taxane particles to the bladder tumor of the subject via intratumoral injection, wherein the taxane particles have a mean particle size (number) of from 0.1 microns to 5 microns, and (b) administering a second administration (second cycle) of an effective amount of the composition to the bladder tumor via intratumoral injection within a periodic interval following the first administration in (a), and (c) optionally, administering a third administration (third cycle) of an effective amount of the composition to the bladder tumor via intratumoral injection within a periodic interval following the second administration in (b), wherein the bladder tumor is eliminated. In some embodiments, the periodic interval is about a week. In some embodiments, the taxane particles are docetaxel particles. In some embodiments, the bladder cancer is a low risk bladder cancer. In other embodiments, the bladder cancer is intermediate risk or high-risk bladder cancer. In some embodiments, the bladder cancer is NMIBC. In other embodiments, the bladder cancer is MIBC.

### EXAMPLES

The present disclosure will be described in greater detail by way of specific examples. The following examples are offered for illustrative purposes only, and are not intended to limit the disclosure in any manner. Those of skill in the art will readily recognize a variety of noncritical parameters, which can be changed or modified to yield essentially the same results.

### Example 1. Production of Paclitaxel Particles and Docetaxel Particles

### Materials and Methods

Raw paclitaxel and docetaxel were purchased from Phyton Biotech (British Columbia, Canada), lot number FP2-15004 and DT7-14025, respectively. Both were characterized in their raw form. The milling of both drugs was accomplished using a Deco-PBM-V-0.41 mill (Deco). The milling conditions for both compounds were as follows:

Ball size=5 mm

RPM=600

Processing time=60 min

Room temperature.

### Preparation of Paclitaxel Particles

A solution of 65 mg/mL of paclitaxel was prepared in acetone. A BETE MicroWhirl® fog nozzle (BETE Fog Nozzle, Inc) and a sonic probe (Qsonica, model number Q700) were positioned in the crystallization chamber approximately 8 mm apart. A stainless steel mesh filter with approximately 100 nm holes was attached to the crystallization chamber to collect the precipitated paclitaxel nanoparticles. The supercritical carbon dioxide was placed in the crystallization chamber of the manufacturing equipment and brought to approximately 1200 psi at about 38° C. and a flow rate of 24 kg/hour. The sonic probe was adjusted to 60% of total output power at a frequency of 20 kHz. The acetone solution containing the paclitaxel was pumped through the nozzle at a flow rate of 4.5 mL/minute for approximately 36 hours. Paclitaxel nanoparticles produced had an average number-weighted mean size of 0.81 μm with an average standard deviation of 0.74 μm over three separate runs.

### Preparation of Docetaxel Particles

A solution of 79.32 mg/mL of docetaxel was prepared in ethanol. The nozzle and a sonic probe were positioned in the pressurizable chamber approximately 9 mm apart. A stainless steel mesh filter with approximately 100 nm holes was attached to the pressurizable chamber to collect the precipitated docetaxel nanoparticles. The supercritical carbon dioxide was placed in the pressurizable chamber of the manufacturing equipment and brought to approximately 1200 psi at about 38° C. and a flow rate of 68 slpm. The sonic probe was adjusted to 60% of total output power at a frequency of 20 kHz. The ethanol solution containing the docetaxel was pumped through the nozzle at a flow rate of 2 mL/minute for approximately 95 minutes). The precipitated docetaxel agglomerated particles and smaller docetaxel particles were then collected from the supercritical carbon dioxide as the mixture is pumped through the stainless steel mesh filter. The filter containing the nanoparticles of docetaxel was opened and the resulting product was collected from the filter.

Docetaxel nanoparticles produced had an average number-weighted mean size of 0.82 μm with an average standard deviation of 0.66 μm over three separate ethanol runs.

### Particle Size Analysis

Particle size was analyzed by both light obscuration and laser diffraction methods. An Particle Sizing Systems AccuSizer 780 SIS system was used for the light obscuration method and Shimadzu SALD-7101 was used for the laser diffraction method. Paclitaxel nanoparticles were analyzed using 0.10% (w/v) sodium dodecyl sulfate (SDS) in water as the dispersant. Docetaxel nanoparticles were analyzed using Isopar G as the dispersant.

Paclitaxel suspensions were prepared by adding approximately 7 mL of filtered dispersant to a glass vial containing approximately 4 mg of paclitaxel particles. The vials were vortexed for approximately 10 seconds and then sonicated in a sonic bath approximately 1 minute. If the sample was already suspended, 1:1 solution of paclitaxel suspension to 0.1% SDS solution was made, vortexed for 10 seconds, and sonicated in the sonic bath for 1 minute.

Docetaxel suspensions were prepared by adding approximately 7 mL of filtered dispersant to a plastic vial containing approximately 4 mg of docetaxel particles. The vial was vortexed for approximately 10 seconds and then sonicated in a sonic bath for approximately 2 minutes. This suspension was used for laser diffraction analysis. Unused suspension was poured into a 125 mL particle-free plastic bottle, which was then filled to approximately 100 mL with filtered dispersant. The suspension was vortex for approximately 10 seconds and then sonicated in the sonic bath for approximately 2 minutes. This diluted suspension was used for light obscuration analysis.

A background test was first performed prior to analyzing particles on the AccuSizer 780 SIS. A new particle-free plastic bottle was filled with blank suspension solution by pumping from a reservoir, using a peristaltic pump, through a 0.22 μm Millipore filter and into the bottle. A background analysis was run to ensure the particle/mL count was below 100 particles/mL. A small amount of paclitaxel suspension, 5-100 depending upon concentration of solution, was pipetted into the plastic bottle in place from the background test and was filled with ˜100 mL dispersant and the analysis was started. Counts were monitored and paclitaxel solution added to reach and/or maintain 6000-8000 particle counts/mL during the entire analysis. Once the analysis was completed, the background data was removed and any measurement with less than four counts was removed.

To analyze particles on SALD-7101 using a batch cell, the analysis was started by choosing Manual Measurement. The refractive index was set as 1.5 to 1.7. The batch cell was filled with filtered dispersant just past the etched line. The blank measurement was ran. A small amount of API (paclitaxel or docetaxel) suspension was pipetted, generally <1 mL, depending upon concentration of solution as low as 100 into the batch cell as needed to achieve an acceptable absorbance between 0.15 and 0.2 absorbance units. The measurements were executed, and the resulting graph with the highest level of confidence was selected; background was automatically accounted for.

### BET Analysis

A known mass between 200 and 300 mg of the analyte was added to a 30 mL sample tube. The loaded tube was then mounted to a Porous Materials Inc. SORPTOMETER®, model BET-202A. The automated test was then carried out using the BETWIN® software package and the surface area of each sample was subsequently calculated.

### Bulk Density Analyte

Paclitaxel or docetaxel particle preparations were added to a 10 mL tared graduated cylinder through a plastic weigh funnel at room temperature. The mass of the drug was measured to a nearest 0.1 mg, the volume was determined to the nearest 0.1 mL and the density calculated.

### Dissolution Studies

### Paclitaxel

Approximately 50 mg of material (i.e.: raw paclitaxel, milled paclitaxel, or paclitaxel particles) were coated on approximately 1.5 grams of 1 mm glass beads by tumbling the material and beads in a vial for approximately 1 hour. Beads were transferred to a stainless steel mesh container and placed in the dissolution bath containing methanol/water 50/50 (v/v) media at 37° C., pH 7, and a USP Apparatus II (Paddle), operating at 75 rpm. At 10, 20, 30, 60, and 90 minutes, a 5 mL aliquot was removed, filtered through a 0.22 μm filter and analyzed on a U(V/V) is spectrophotometer at 227 nm. Absorbance values of the samples were compared to those of standard solutions prepared in dissolution media to determine the amount of material dissolved.

### Docetaxel

Approximately 50 mg of material (i.e.: raw docetaxel, milled docetaxel, or docetaxel particles) was placed directly in the dissolution bath containing methanol/water 15/85 (v/v) media at 37° C., pH 7, and a USP Apparatus II (Paddle), operating at 75 rpm. At 5, 15, 30, 60, 120 and 225 minutes, a 5 mL aliquot was removed, filtered through a 0.22 μm filter, and analyzed on a UV/VIS spectrophotometer at 232 nm. Absorbance values of the samples were compared to those of standard solutions prepared in dissolution media to determine the amount of material dissolved.

### Results

The BET surface area of particles produced using the above protocol and variations thereof (i.e.: modifying nozzles, filters, sonic energy sources, flow rates, etc.) ranged between 22 and 39 m²/g. By comparison, the BET surface area of raw paclitaxel was measured at 7.25 m²/g, while paclitaxel particles made according to the methods of U.S. Pat. Nos. 5,833,891 and 5,874,029 ranged from 11.3 to 15.58 m²/g. Exemplary particle sizes produced using the methods of the disclosure are shown in Table 1.

TABLE 1

| | | |
|---|---|---|
| | Mean Size | St Dev |
| Surface | μm | μm |

| | area m²/g | Number | Volume | Number | Volume |
|---|---|---|---|---|---|
| 1 | 38.52 | 0.848 | 1.600 | 0.667 | 0.587 |
| 2 | 33.82 | 0.754 | 0.988 | 0.536 | 0.486 |
| 3 | 35.90 | 0.777 | 1.259 | 0.483 | 0.554 |
| 4 | 31.70 | 0.736 | 0.953 | 0.470 | 0.466 |
| 5 | 32.59 | 0.675 | 0.843 | 0.290 | 0.381 |
| 6 | 38.22 | 0.666 | 0.649 | 0.344 | 0.325 |
| 7 | 30.02 | 0.670 | 0.588 | 0.339 | 0.315 |
| 8 | 31.16 | 0.672 | 0.862 | 0.217 | 0.459 |
| 9 | 23.90 | 0.857 | 1.560 | 0.494 | 0.541 |
| 10 | 22.27 | 0.857 | 1.560 | 0.494 | 0.541 |
| 11 | 26.19 | 0.861 | 1.561 | 0.465 | 0.546 |

Comparative studies on bulk density, SSA, and dissolution rates (carried out as noted above) for raw drug, milled drug particles, and drug particles produced by the methods of the present disclosure are provided in Table 2 and Table 3 below. The full dissolution time course for the paclitaxel and docetaxel materials are provided in Table 4 and Table 5, respectively.

TABLE 2
Compound: Paclitaxel

| | Raw | Particles | | | |
|---|---|---|---|---|---|
| Characteristic | Material | Batch 1 | Batch 2 | Mean | Milled |
| Number Mean (um) | 1.16 | 0.83 | 0.67 | 0.75 | 0.89 |
| Volume Mean (um) | 1.29 | 1.42 | 0.57 | 1.00 | 1.35 |
| Bulk Density (g/cm³) | 0.26 | 0.060 | 0.11 | 0.085 | 0.31 |
| Surface Area (m²/g) | 10.4 | 35.6 | 39.8 | 37.7 | 15.0 |
| Dissolution (30 min) | 18% | 42% | 52% | 47% | 32% |

TABLE 3
Compound: Docetaxel

| | Raw | Particles | | | |
|---|---|---|---|---|---|
| Characteristic | Material | Batch 1 | Batch II | Mean | Milled |
| Number Mean (um) | 1.58 | 0.92 | 0.80 | 0.86 | 1.11 |
| Volume Mean (um) | 5.05 | 4.88 | 4.03 | 4.46 | 3.73 |
| Bulk Density (g/cm³) | 0.24 | 0.062 | 0.096 | 0.079 | 0.44 |
| Surface Area (m²/g) | 15.9 | 43.0 | 45.4 | 44.2 | 15.2 |
| Dissolution (30 min) | 11% | 27% | 27% | 27% | 9% |

TABLE 4
Paclitaxel Dissolution time course

| Timepoint | Paclitaxel | Paclitaxel | Milled |
|---|---|---|---|
| (minutes) | Raw Material | Particles | Paclitaxel |
| 0 | 0.0% | 0.0% | 0.0% |
| 10 | 14.0% | 40.2% | 23.0% |
| 20 | 17.8% | 47.6% | 30.0% |
| 30 | 18.4% | 51.9% | 32.3% |
| 60 | 23.9% | 58.3% | 38.6% |
| 90 | 28.6% | 62.9% | 43.5% |

TABLE 5
Docetaxel Dissolution time course

| Timepoint | Docetaxel | Docetaxel | Milled |
|---|---|---|---|
| (minutes) | Raw Material | Particles | Docetaxel |
| 0 | 0.0% | 0.0% | 0.0% |
| 5 | 3.2% | 12.1% | 3.2% |
| 15 | 6.9% | 21.7% | 5.9% |
| 30 | 11.2% | 27.2% | 9.3% |
| 60 | 16.4% | 32.9% | 12.2% |
| 120 | 22.4% | 38.9% | 13.6% |
| 225 | 26.8% | 43.1% | 16.0% |

### Example 2. Pilot Evaluation Study of Direct Injection of Dye into Rabbit Bladder Wall

A study was conducted to evaluate the direct injection of blue tissue dye in a vehicle formulation into the bladder wall (intramural injection) of rabbits. Injection into the bladder wall is the intended route of administration of nDoce (nanoparticulate docetaxel as disclosed herein, approximately 99% docetaxel with a mean particle size (number) of 1.078 microns, a SSA of 37.2 m²/g, and a bulk density (not tapped) of 0.0723 g/cm³ used in this example) suspension in humans. The rabbit is an appropriate species for study due to the similarity of bladder wall musculature and size of bladder. The formulation vehicle contained 0.4% w/w polysorbate 80 NF, 3.2% w/w ethanol (200 proof), prepared in sterile normal saline solution (0.9% Sodium Chloride for Injection, USP). An amount of 10 mg/mL of Evans Blue tissue dye was added to the vehicle, which was adequate for visualization.

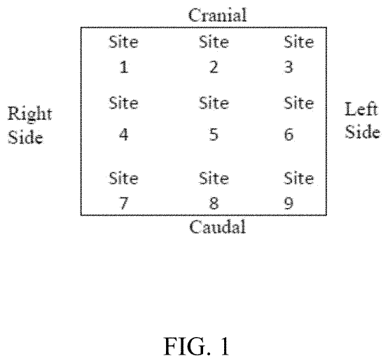

On Day 1, following induction of anesthesia, animals were placed in dorsal recumbency and prepared for sterile surgery. Following a midline incision, the urinary bladder was accessed and the vehicle containing Evans Blue tissue dye was injected into the bladder wall, using a 25 or 27 gauge needle; care was taken not to enter the urinary bladder. The needle was inserted bevel up for the injections, and then rotated to the bevel down position prior to removing the needle from the bladder wall. Each animal received nine injections according to the dosing scheme shown in FIG. 1 with a dose volume of 100 μL per injection site. The injections were space approximately 7 mm apart. Following each injection, photographs were obtained. At study termination (Day 8), the animals were euthanized. The skin was reflected from a ventral midline incision and any gross findings were identified and correlated with antemortem findings. The bladder was examined (externally and internally) to determine if the dosing procedure was well-tolerated.

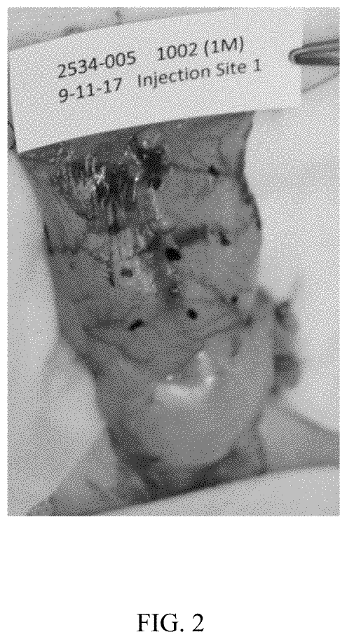

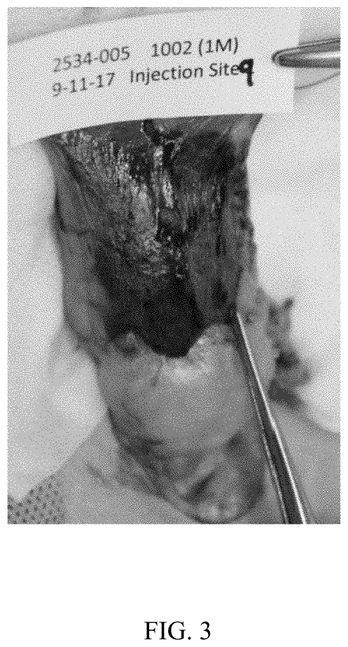

Photographs obtained after the 1^{st} injection and following the 9^{th} injection of one animal are shown in FIG. 2 and FIG. 3 respectively. As can be seen in the photographs, the blue dye diffused to encompass the entire area circumscribed by the injections sites and provided confluent coverage of the entire test site. The injection procedure did not produce any unexpected adverse effects.

### Example 3. Single Dose Intravesicular Range-Finding Toxicity and Toxicokinetic Study of nDoce in Rats

A non-GLP study was conducted to evaluate and characterize the toxicity and toxicokinetics of the test article, nDoce (nanoparticulate docetaxel as disclosed herein, approximately 99% docetaxel with a mean particle size (number) of 1.078 microns, a SSA of 37.2 m²/g, and a bulk density (not tapped) of 0.0723 g/cm³ used in this example), and to estimate the maximum tolerated dose (MTD) following a single intravesicular administration into the bladder in CD® [Crl:CD® (SD)] female rats. The intravesicular route is the intended route of administration of nDoce suspension in humans. The rat is the usual rodent model used for evaluating the toxicity of various classes of chemicals and for which there is a large historical database. Female rats were selected for this study given the ease of urethral catheterization relative to male rats. Animals were maintained for a 72-hr or 14-day postdose recovery period. Rats were assigned to the study as indicated in Table 6 and Table 7 below.

TABLE 6
Study Design - Main Study

| | | Dose | | | | |
|---|---|---|---|---|---|---|
| | Dose | Concen- | Dose | Number | Necropsy (Post | |
| | Level | tration | Volume | of | Injection Interval) | |
| Group | (mg/ | (mg/ | (mL/ | Female | 72 hours^{b} | Day 15^{c} |
| Number | kg) | mL) | kg) | Animals | (Terminal) | (Recovery) |
| 1 | 0^{a} | 0 | 2 | 6 | 3 | 3 |
| 2 | 3.2 | 1.6 | 2 | 6 | 3 | 3 |
| 3 | 10 | 5 | 2 | 6 | 3 | 3 |
| 4 | 30 | 15 | 2 | 6 | 3 | 3 |
| 5 | 80 | 40 | 2 | 6 | 3 | 3 |

^{a}Administered the vehicle control, 0.40% (w/w) Polysorbate 80 NF, 3.2% (w/w) Dehydrated Alcohol (Ethanol), 200 Proof, Undenatured USP, in 0.9% Sodium Chloride for Injection, USP.
^{b}Last three animals per group.
^{c}First three animals per group.

TABLE 7
Study Design - Toxicokinetic

| | | | | | Necropsy^{b} | | |
|---|---|---|---|---|---|---|---|
| | Dose | Dose | Dose | Number | (Post Injection Interval) | | |
| Group | Level | Concentration | Volume | of Female | Cohort 1 | Cohort 2 | Cohort 3 |
| Number | (mg/kg) | (mg/mL) | (mL/kg) | Animals | (n = 3) | (n = 3) | (n = 3) |
| 6 | 0^{a} | 0 | 2 | 2 | 4 hours^{c} | NA | 168 hours^{c} |
| 7 | 3.2 | 1.6 | 2 | 9 | 1, 4, 8 | 24, 36, | 72, 96, |
| | | | | | hours | 48 hours | 168 hours |
| 8 | 10 | 5 | 2 | 9 | 1, 4, 8 | 24, 36, | 72, 96, |
| | | | | | hours | 48 hours | 168 hours |
| 9 | 30 | 15 | 2 | 9 | 1, 4, 8 | 24, 36, | 72, 96, |
| | | | | | hours | 48 hours | 168 hours |
| 10 | 80 | 40 | 2 | 9 | 1, 4, 8 | 24, 36, | 72, 96, |
| | | | | | hours | 48 hours | 168 hours |

^{a}Administered the vehicle control, 0.40% (w/w) Polysorbate 80 NF, 3.2% (w/w) Dehydrated Alcohol (Ethanol), 200 Proof, Undenatured USP, in 0.9% Sodium Chloride for Injection, USP.
^{b}Examination of the urinary bladder only.
^{c}One control toxicokinetic animal per time point.
NA—Not applicable.

The nDoce (nanoparticulate docetaxel) suspensions were prepared by mixing the appropriate amount of nDoce (nanoparticulate docetaxel) powder with the sterile reconstitution solution [1% w/w Polysorbate 80 NF, 8% w/w Ethanol 200 proof (Dehydrated Alcohol Undenatured USP) in normal saline solution (0.9% Sodium Chloride for Injection USP)], and normal saline solution (0.9% Sodium Chloride for Injection USP) to obtain nominal docetaxel particle concentrations of 1.6, 5, 15, and 40 mg/mL. The mean particle size (number) of the nDoce powder was 1.0 micron. The vehicle control was prepared by adding the appropriate amount of normal saline solution (0.9% Sodium Chloride for Injection USP) to the appropriate amount of the sterile reconstitution solution (see above) to give a final concentration of 0.4% w/w Polysorbate 80 and 3.2% w/w Ethanol in the vehicle control.

The nDoce suspension was administered once on Day 1, via intravesicular instillation, into the bladder at a total dose volume of 2 mL/kg. The dose levels were 0 (control), 3.2, 10, 30, and 80 mg/kg. Prior to instillation, the animals were anesthetized and a rat bladder catheter was advanced into the bladder via the urethra. Once the catheter was in the bladder, the contents of the bladder were evacuated to empty the bladder of urine. The nDoce suspension or vehicle control was instilled through the catheter into the bladder as a bolus injection and held in the bladder for 2 hours. At 2 hours postdose, the contents of the bladder were collected through the catheter.

Assessment of toxicity was based on mortality, clinical observations, body weight, food consumption, and clinical and anatomic pathology of selected tissues. There were no nDoce related findings in the following parameters evaluated: mortality, clinical observations, body weights, food consumption, or urinalysis. Minimal to mild chronic-active inflammation was noted in the bladder of two of three rats in Group 5 (80 mg/kg) at the 72 hour terminal interval. However, the severity was of low grade and was reversible, as it was not observed at the end of the recovery interval period (Day 15). Therefore, 80 mg/kg is considered the MTD given that the observed effects were minimal in severity, and did not persist to the end of the recovery period.

### Example 4. Human Bladder Cancer (UM-UC-3) Mouse Xenograft Study

A study was conducted to evaluate the effect of 1, 2, and 3 weekly intratumoral injection (IT) administrations (administration cycles) of nDoce (nanoparticle docetaxel as disclosed herein, approximately 99% docetaxel with a mean particle size (number) of 1.078 microns, a SSA of 37.2 m²/g, and a bulk density (not tapped) of 0.0723 g/cm³ used in this example) suspension on growth of subcutaneous (SC) UM-UC-3 bladder cancer cell line (ATCC-CRL-1749) tumors in immunocompromised (Hsd:Athymic Nude-Foxn1nu nude) mice. Intratumoral injection administration of a vehicle and intravenous (IV) administration of docetaxel solution were also incorporated into the study as control groups.

Tumors were implanted with 1×10⁷ cells (1004, volume) into right flank (PBS 1:1 with matrigel:BD356234). Tumor volume was determined with calipers. Formula: V=(r length*r width*r height)*π*4/3. Animals were weighed 2×/week. Tumor volumes were determined every 3 to 4 days following tumor implant (total of −20 measurements) and on day of euthanasia. Photo images of tumors were obtained at 2, 3 and 4 weeks post implantation and on day of euthanasia. Animals were euthanized once the tumor reached a size of 3,000 mm³ or up to the point of significant tumor ulceration. At the time of euthanasia, tumors were isolated and halved. One half of the tumor was flash frozen in LN2 stored at −80° C. and will subsequently be analyzed. The second half of the tumor was fixed in formalin. Two H&E stained slides/tumor were prepared (up to 4 tumor/group were processed).

At day 18 after tumor implant, when average tumor size was between 50-325 mm³, animals were sorted into five groups with equal average tumor sizes and were treated as shown in Table 8 below.

TABLE 8
Main Study Design

| | | | Weekly | |
|---|---|---|---|---|
| Group | Name | Treatment | Admin Cycles | n |
| A | Vehicle IT | Vehicle (IT) | 3 | 10 |
| | 3 cycles | 63 μl/tumor | | |
| B | Docetaxel IV | Docetaxel Solution | 3 | 9 |
| | 3 cycles | 30 mg/kg (IV) | | |
| | | Docetaxel = 3 mg/mL | | |
| C | nDoce IT | nDoce Suspension | 1 | 10 |
| | 1 cycle | 100 mg/kg (IT) | | |
| | | nDoce = 40 mg/mL; | | |
| | | 63 μl/tumor | | |
| | | (2.5 mg nDoce) | | |
| D | nDoce IT | nDoce Suspension | 2 | 9 |
| | 2 cycles | 100 mg/kg (IT) | | |
| | | nDoce = 40 mg/mL; | | |
| | | 63 μl/tumor | | |
| | | (2.5 mg nDoce) | | |
| E | nDoce IT | nDoce Suspension | 3 | 9 |
| | 3 cycles | 100 mg/kg (IT) | | |
| | | nDoce = 40 mg/mL; | | |
| | | 63 μl/tumor | | |
| | | (2.5 mg nDoce) | | |

For IT administration (Vehicle/nDoce), injections (using 27 G, ½″ needle) were administered at three sites within the tumor (total calculated injection volume based on 40 mg/mL nDoce stock and 25 g mouse=63 μL; split evenly across the three injection sites) to maximize distribution of the test formulation throughout the tumor. The second treatments (2^{nd} cycle) occurred 7 days following first treatment (1′ cycle) and third treatments (3^{rd} cycle) occurred 14 days following the first treatment. The docetaxel solution IV was administered via the tail vein.

The test formulations were prepared as follows:

Vehicle (Control): Diluted 1 ml of the 1% Polysorbate 80/8% Ethanol in normal saline (0.9% Sodium Chloride for Injection) reconstitution solution with 1.5 mL of normal saline (0.9% Sodium Chloride for Injection, USP). The final concentration of polysorbate 80 was 0.4% and the final concentration of ethanol was 3.2% in the Vehicle.
nDoce Suspension: Added 1 ml of the 1% Polysorbate 80/8% Ethanol in normal saline (0.9% Sodium Chloride for Injection) reconstitution solution into the vial of nDoce particles powder (100 mg/60 cc vial). The mean particle size (number) of the nDoce particles powder was 1.0 micron. Vigorously hand shook the vial with inversions for 1 minute. Immediately after shaking, added 1.5 ml of normal saline solution (0.9% Sodium Chloride for Injection USP) to the vial and hand shook the vial for another 1 minute to make a 40 mg/mL suspension. Allowed the suspension to sit undisturbed for at least 5 minutes to reduce entrapped air and foam.
Docetaxel Solution: Prepared a 20 mg/mL docetaxel stock solution in 50% Ethanol/50% Polysorbate 80. Added normal saline solution (0.9% Sodium Chloride for Injection) to stock solution to make a final, 3 mg/mL docetaxel solution. Vortexed to mix.

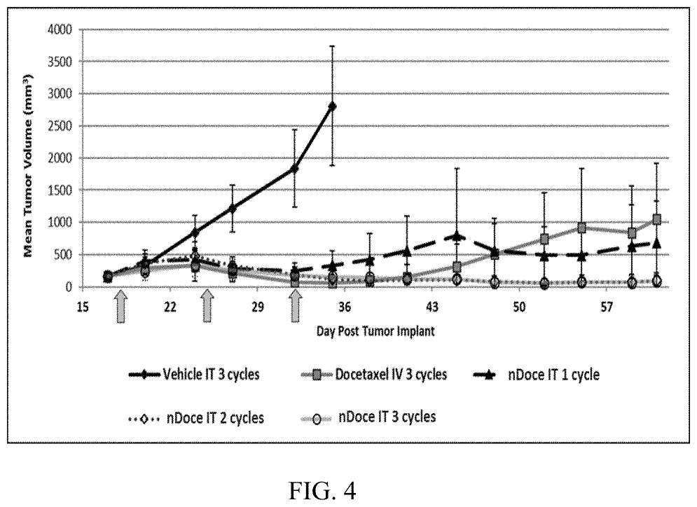

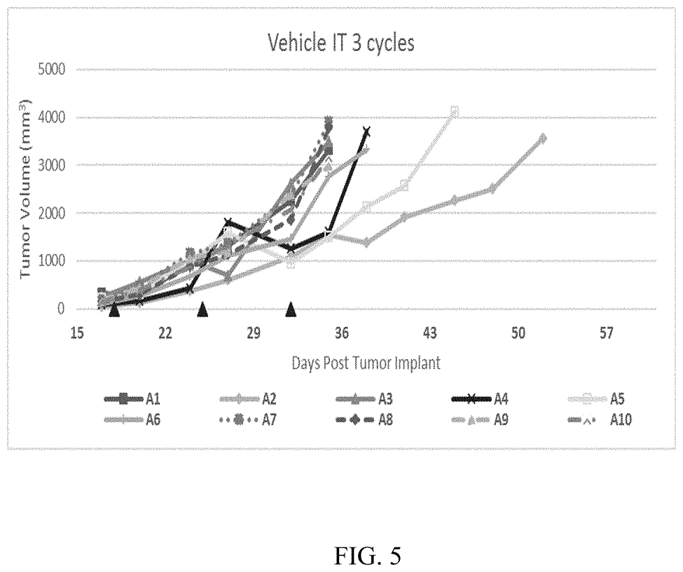

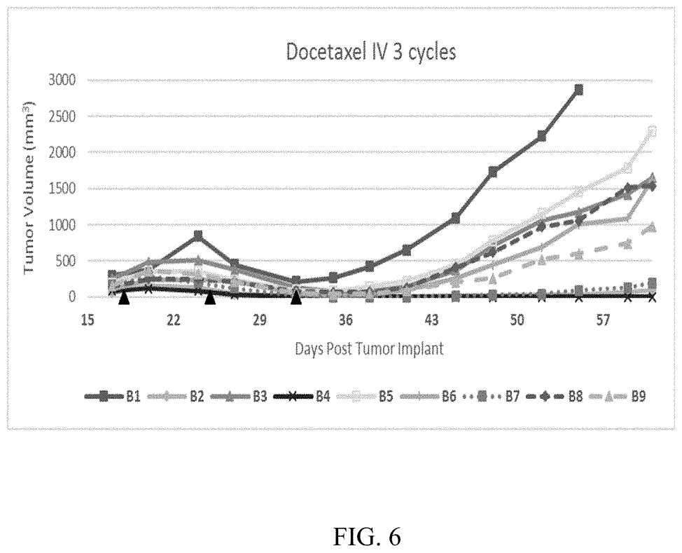

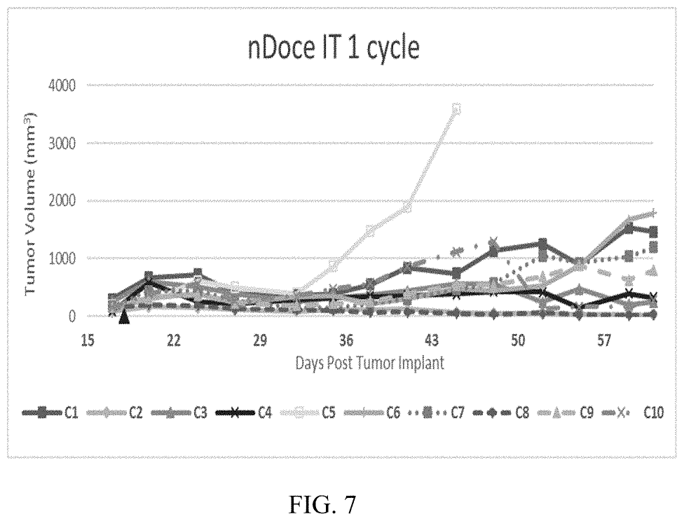

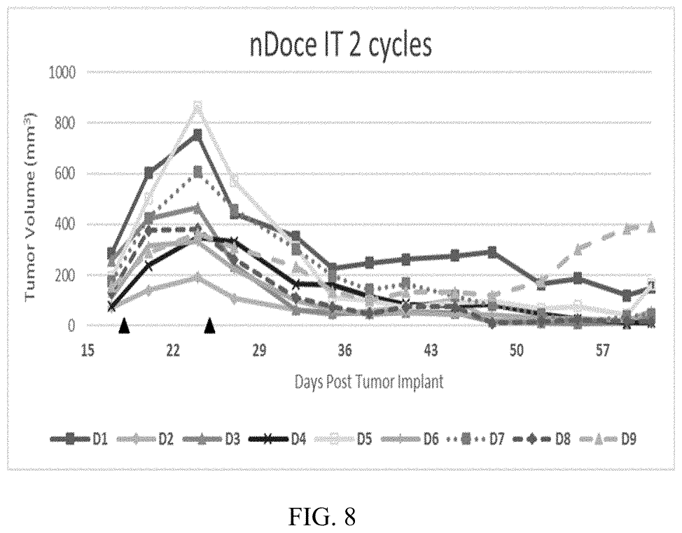

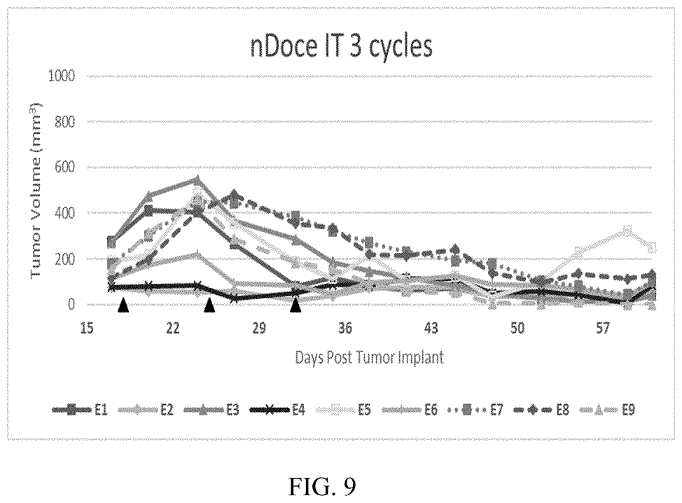

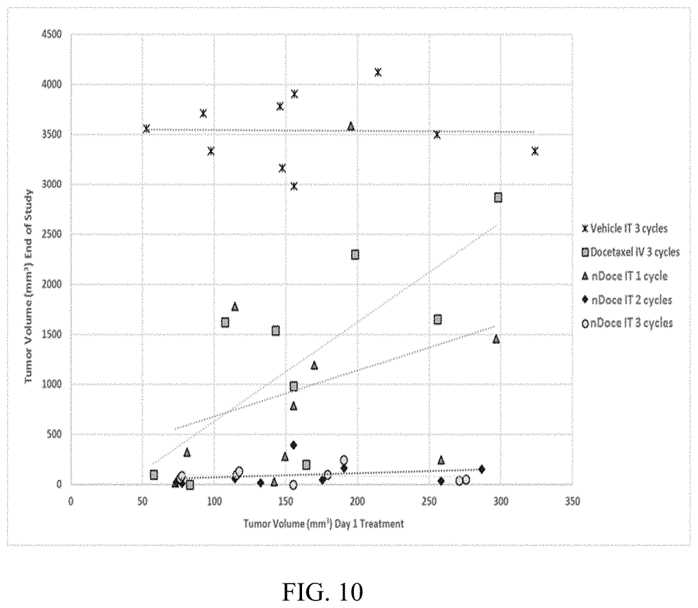

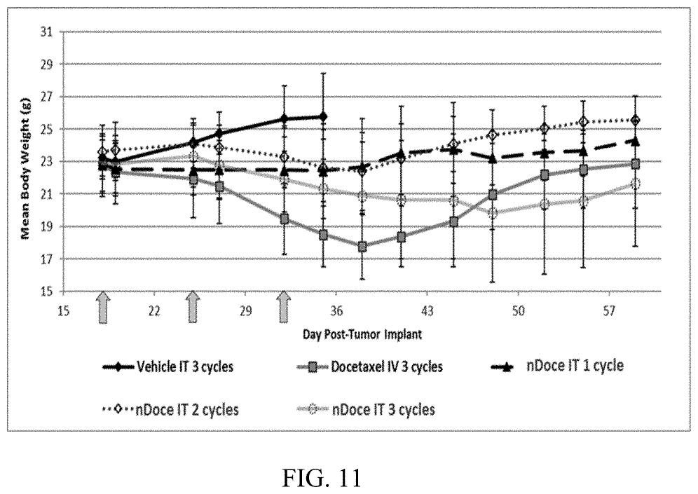

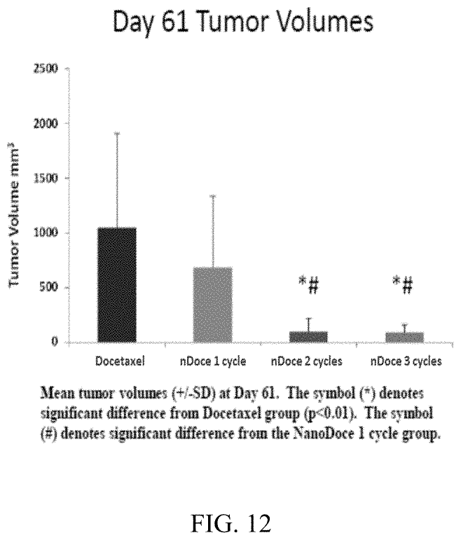

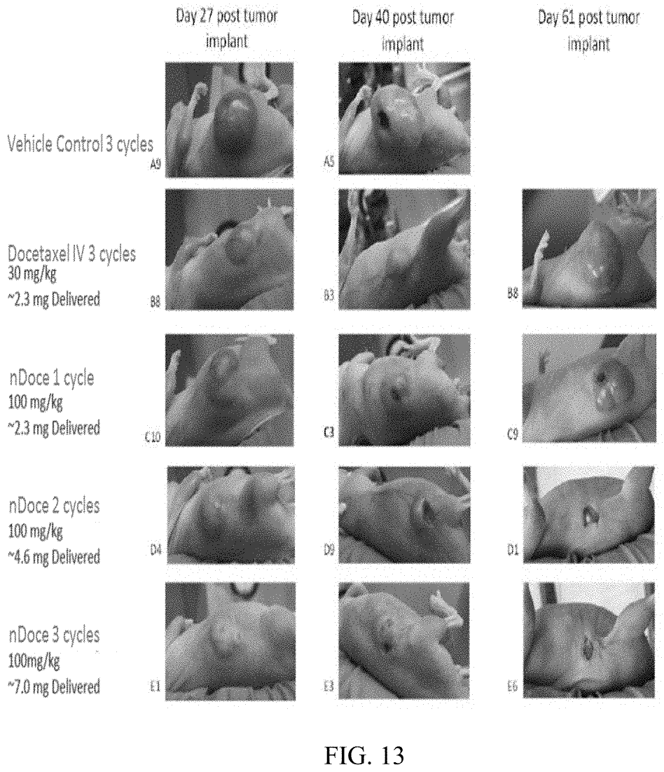

Tumor volumes were determined 2×/week for the duration of the study (61 days). The results of the study are shown in FIG. 4, FIG. 5, FIG. 6, FIG. 7, FIG. 8, FIG. 9, FIG. 10, FIG. 11, FIG. 12 & FIG. 13. As seen in FIG. 4, tumor volumes decreased and tumors were effectively eliminated for dosages of nDoce IT 2 cycles and nDoce IT 3 cycles. Tumor volumes decreased initially for dosages of nDoce IT 1 cycle and Docetaxel IV 3 cycles, but subsequently increased. These observations are also reflected in FIG. 5, FIG. 6, FIG. 7, FIG. 8, FIG. 9, FIG. 12 & FIG. 13.

The scatter plot in FIG. 10 shows tumor volumes per animal on Day 1 of treatment vs. end of study (day of sacrifice). As can be seen in FIG. 10, the volume of the tumor in a given animal at the end of study was not dependent upon the initial size of the tumor of the same animal for the animals treated with nDoce IT 2 cycles and nDoce IT 3 cycles, as essentially all the tumors were effectively eliminated. However, for animals treated with Docetaxel IV 3 cycles, the volume of the tumor at the end of the study was generally dependent upon the initial tumor volume for a given animal, i.e., the larger the initial tumor volume, the larger the tumor volume at the end of the study. The treatment with Docetaxel IV 3 cycles was somewhat effective at treating small tumors, but not very effective in treating large tumors. Administering nDoce IT (intratumorally) for 2 cycles or 3 cycles effectively treated the tumors regardless of the initial tumor size.

As can be seen in FIG. 11, the initial animal weight loss for animals treated with Docetaxel IV 3 cycles was generally greater than that of animals treated with nDoce IT 1 cycle, nDoce IT 2 cycles, and nDoce IT 3 cycles. Weights eventually recovered to some degree in all treatments. This may suggest that the side effect of initial appetite loss is greater with Docetaxel IV administration than with nDoce IT administrations. It was also observed that animals treated with Docetaxel IV 3 cycles had greater signs of peripheral neuropathy than did those treated with nDoce IT 3 cycles, and no signs of peripheral neuropathy were observed in those treated with nDoce IT 1 cycle or 2 cycles.

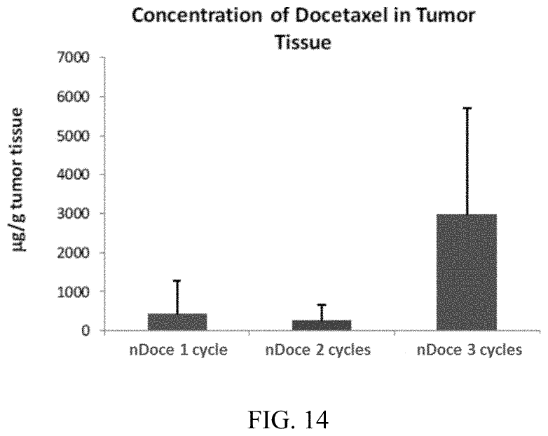

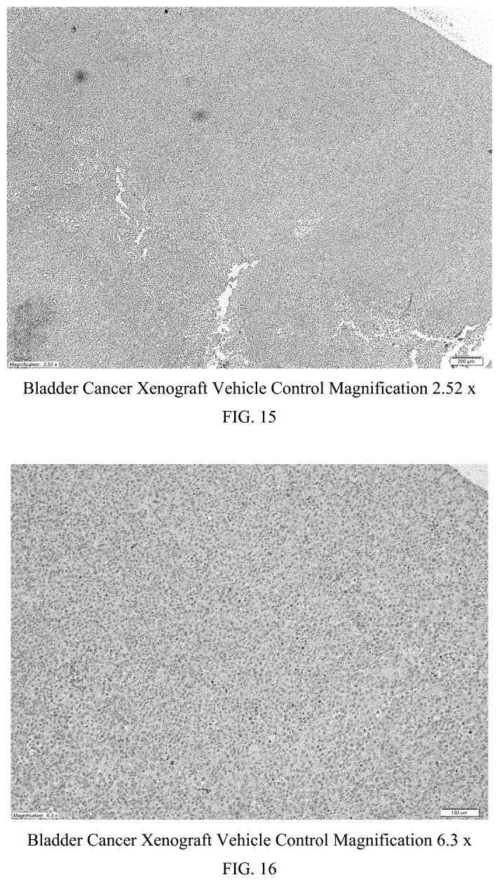

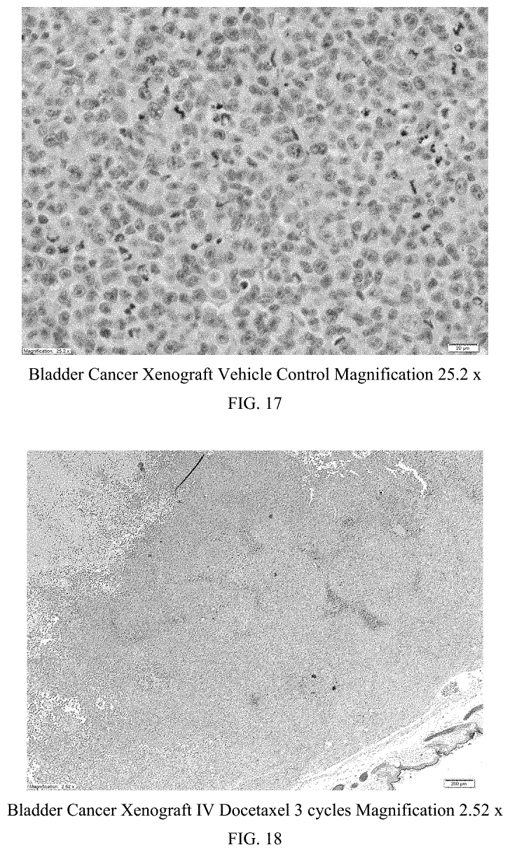

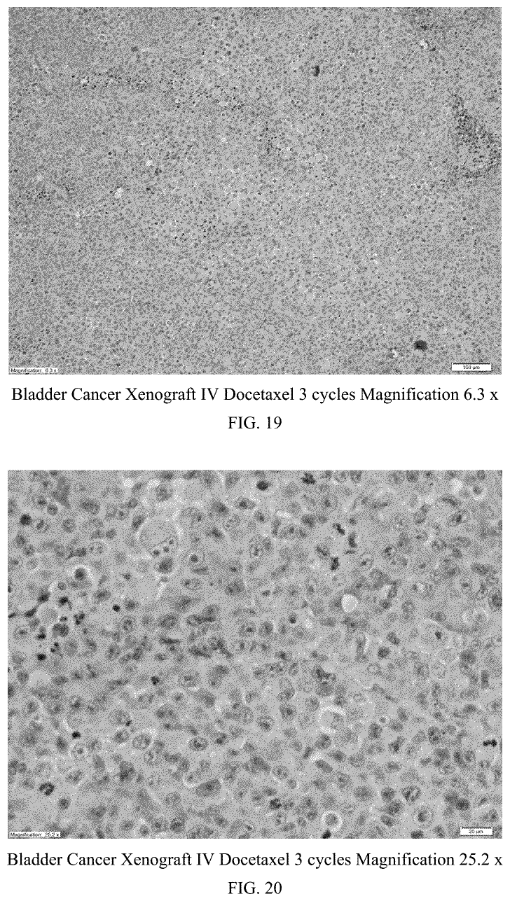

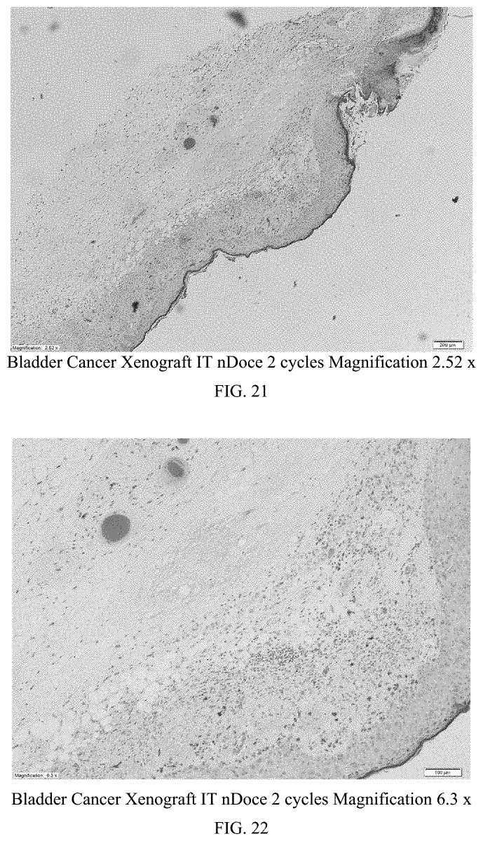

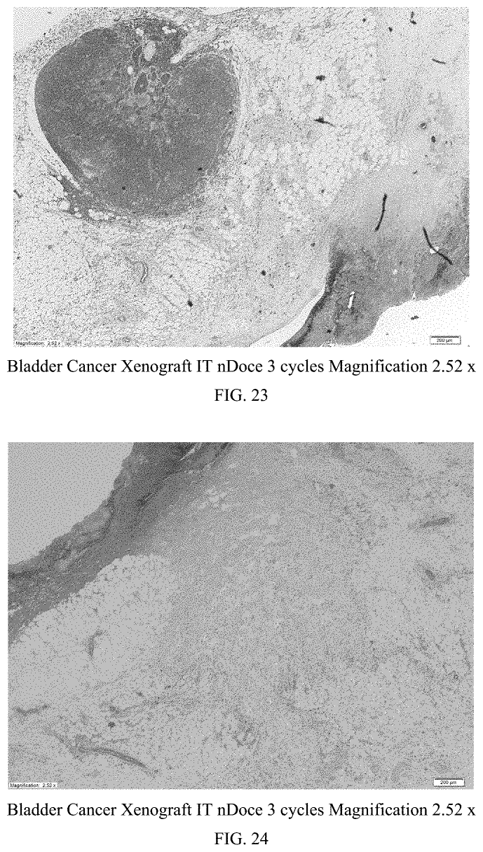

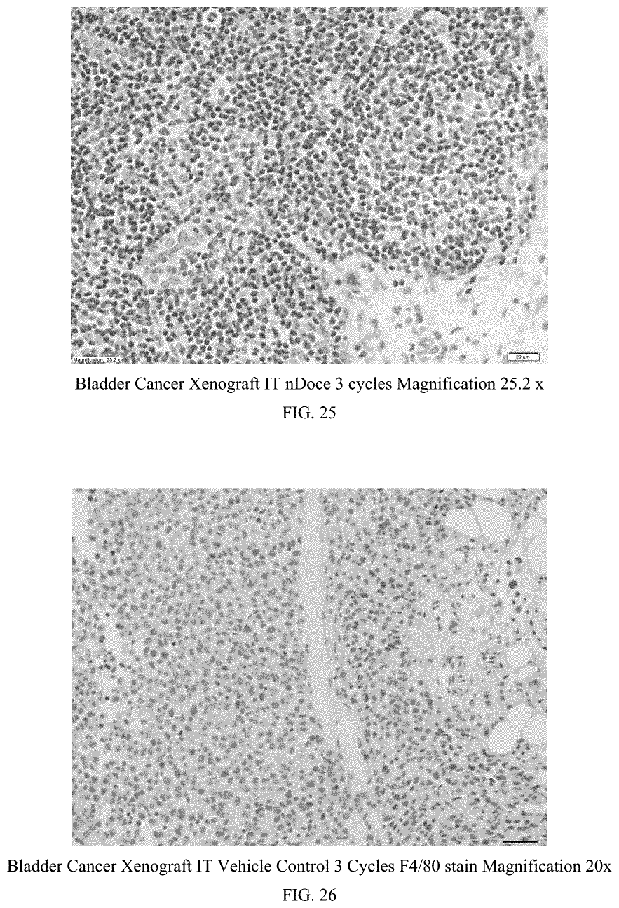

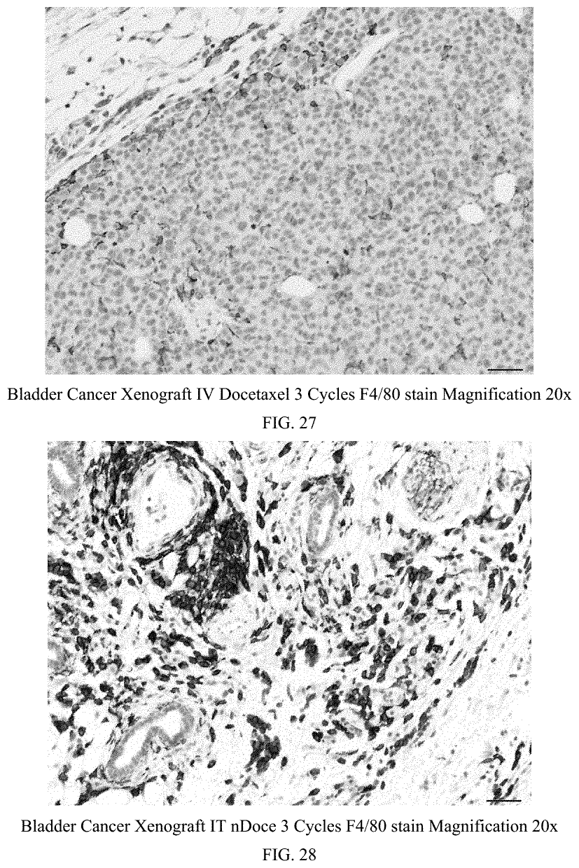

On the day of death or euthanasia, tumor tissues samples were collected and frozen in LN2 for docetaxel analysis, histology, and immunohistochemistry (IHC) observations. In the IV docetaxel control group, only 1 tumor (of 7 measured) had docetaxel levels above the limit of quantitation of the assay (1 ng/g). Measurable levels of docetaxel were found in all tumors from the IT nDoce groups with the nDoce 3 cycle group tending to have the highest concentrations of docetaxel remaining in the tumors (see FIG. 14). Photomicrographs of histology slides, H&E stain, are shown in FIGS. 15 to 25. Photomicrographs of IHC slides stained with F4/80 antibody stain are shown in FIG. 26, FIG. 27, and FIG. 28.

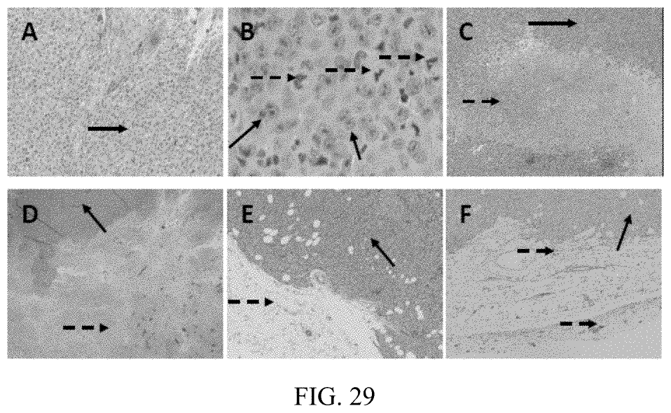

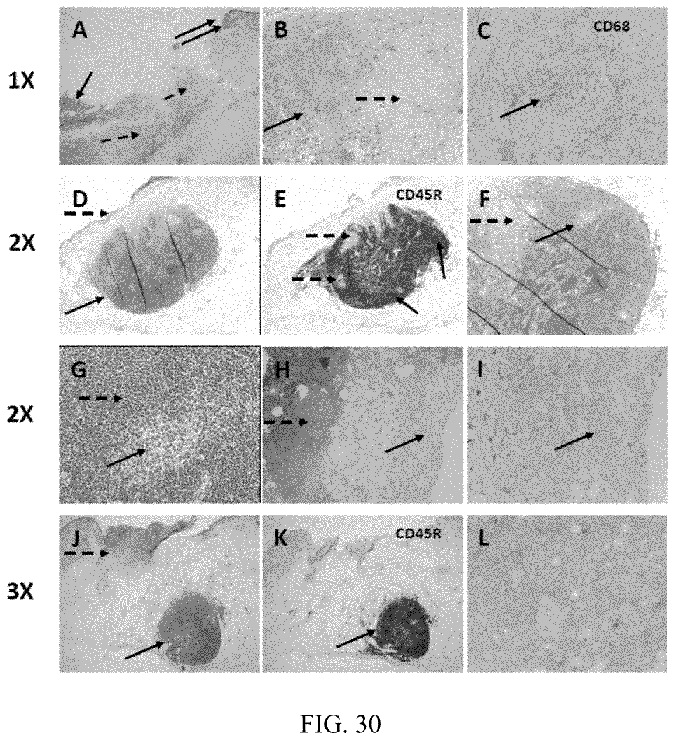

Additional H&E and Immunohistochemical (IHC) evaluations were conducted on formalin-fixed tissue and are shown in FIG. 29 and FIG. 30.

### Histological Overview of Photomicrographs in FIGS. 15 to 25

### General Observations:

Control: Extensive levels of viable tumor with proliferating cells and little to no mononuclear immune cell infiltration, occasional macrophages noted.

Docetaxel Solution: many viable appearing tumor masses with some macrophage and occasional lymphocytic response along with some tumor necrosis.

nDoce 2 cycles: Some remaining isolated tumor cells, small area of skin injury, scar/fibrosis seen, immune cell infiltrate including macrophages and mononuclear cells.

nDoce 3 cycles: Some remaining isolated tumor cells, small area of skin injury, scar/fibrosis seen, immune cell infiltrate including macrophages and mononuclear cells

Extensive mononuclear cell infiltration was observed at the site of tumor implantation in the subcutaneous space in animals receiving intratumoral injection of nDoce. With increased numbers of cycles, there is increased tumor response, but there is some skin injury, perhaps due to the small space and shallow area for injection on the flank of a nude mouse (e.g., tumor right up against skin that is tightly drawn over the tumor). As the model used is T cell deficient, it is likely that the lymphocytic cells are B cells or NK cells. B cells are responsible for the production of cytotoxicity (the antibodies bind to cells expressing Fc Receptors and enhance the killing ability of these cells. NK cells are innate lymphoid cells that are crucial in the killing of tumor cells. In patients with tumors, NK cell activity is reduced allowing for the growth of the tumor. Along with T cells, NK cells are the target of some check point inhibitors to increase their activity. In all histological samples provided, macrophages were present in the tumor, but the number did not appear to significantly increase.

By the use of a wide array of surface receptors capable of delivering either triggering or inhibitory signals, NK cells can monitor cells within their environment to ascertain if the cell is abnormal (tumor or virally infected) and should be eliminated through cytotoxicity. The cytotoxicity and chemotaxis of NK cells can be modified by many pathological processes including tumor cells and their byproducts. In response to certain signals their functions are enhanced or potentiated. In response to several Pathogen Associated Molecular Patterns (PAMPs) by using different Toll Like Receptors (TLR); NK cells can increase cytokine production and/or cytolytic activity. Cytokines, including IL-2, IL-15, IL-12, IL-18, and IFNs α/β can also modify the activity of NK cells. NK cells are not simple cells that are only cytolytic effectors capable of killing different tumor cell targets; rather, they represent a heterogeneous population which can finely tune their activity in variable environmental contexts.

The tumor burden is significantly reduced in the site of xenograft injection in the animals treated with nDoce and the intratumoral injection is more effective than intravenous docetaxel. Therefore, the localized administration of docetaxel in the form of nDoce provides additional potency. This is likely due to both the longer exposure to the chemotherapy over time and the vigorous cellular infiltration to the site of the tumor. This latter response appeared to be dependent on the dose density (actual dose and dose frequency). Anatomically, macrophages are present at high numbers at the margins of tumors with decreasing frequency throughout the stroma moving deeper within the tumor.

### Immunohistochemistry Overview of FIG. 26, FIG. 27, and FIG. 28

FIG. 26: Vast sheet of viable tumor cells and no mononuclear immune cells (no brown staining).

FIG. 27: Very little tumor cell destruction and few scattered mononuclear immune cells among vast number of viable tumor cells.

FIG. 28: Virtually no tumor cells left and vast numbers of mononuclear immune cells organized into distinct patterns (likely mostly macrophages).

### Additional H&E and Immunohistochemical (IHC) Evaluation (See FIG. 29 and FIG. 30)

Tumor tissue was fixed before H&E and IHC staining. Bladder tissue sections were deparaffinized and processed by standard H&E and IHC staining. At least four tumors per treatment group were processed.

Observations: FIG. 29 Control Cases:

Top row: H&E Stained Sections (A-C): (A) Bladder carcinoma composed of sheets of closely packed large pleomorphic tumor cells. (B) Higher power view showing large tumor cells with prominent nucleoli (solid arrows) and a marked increase in mitotic figures (dashed arrows). (C) Low power view showing a focus of geographic tumor cell necrosis with admixed degenerating tumor cells (dashed arrow) and adjacent viable carcinoma at bottom and top of image (solid arrow).
Bottom row: IT vehicle (D) and IV Docetaxel (E and F): (D) IT vehicle case (case A3). H&E stained section showing extensive necrosis in bottom half of image (dashed arrow) and viable carcinoma in top left (solid arrow). (E) IV docetaxel (case B1). H&E stained section showing viable carcinoma in top right portion of image that appeared similar to that in the control and IT vehicle cases (solid arrow). Note sharp demarcation from non-neoplastic fatty tissue in lower left without a capsule surrounding the tumor (dashed arrow). The fat contained a sparse immune cell infiltrate. (F) IV docetaxel (case B1). CD68 stain highlighting mild macrophage infiltrate in surrounding stroma in bottom half of image (dashed arrows). Viable carcinoma is at top of image (solid arrow).

Observations: FIG. 30 Intratumoral nDoce cases (representative images from all groups included: 1 cycle, 2 cycles and 3 cycles).

Top row: One cycle nDoce (1×) (case C4). (A) Low power H/E staining showing extensive geographic tumor cell necrosis consisting of homogeneous eosinophilic staining of non-viable necrotic material (dashed arrows). The necrosis spans from the overlying mouse skin surface in top right of image (two solid arrows) to the focal viable carcinoma in the bottom left corner (single solid arrow). (B) High power view of viable carcinoma at left (solid arrow) and necrosis at right (dashed arrow). (C) CD68 immunohistochemical stain showing mild macrophage infiltrate (solid arrow) in the surrounding non-neoplastic fatty tissue.
Second row: Two cycles of nDoce treatment (2×) (case D2). (D) Low power view showing a tertiary lymphoid structure (TLS) that measured 2 mm in maximum dimension (solid arrow). Note well-circumscribed border of TLS and demarcation from surrounding tissue with immune cell infiltrate. Note overlying ulcerated skin (dashed arrow). (E) CD45R immunostain (B-cell marker) showing extensive staining throughout the TLS, confirming that the majority of the lymphocytes in the TLS are B-cells. Note the organization into B-cell lymphoid follicles (solid arrows) and focal unstained areas that represent interfollicular “T-cell” zones (dashed arrows). (F) Higher power view of same TLS. Note the organization of the TLS with a hilar region that contains medullary sinuses (dashed arrow) and a germinal center forming in one of the lymphoid follicles (solid arrow).
Third row: Two cycles of nDoce treatment (2×) (case D2), continued. (G) Higher power view of germinal center. Note the polymorphous lymphoid population in the germinal center that consists of a mixed population of small mature lymphocytes, intermediate sized centrocytes and occasional larger centroblasts (solid arrow). Compare this with the adjacent homogenous population of small mature lymphocytes (dashed arrow). (G) Same case, showing separate area with ulcerated skin at left (dashed arrow) and necrotic tissue at right (solid arrow). No viable carcinoma is present. (H) Higher power view of the necrotic area showing homogenous eosinophilic amorphous necrotic material with no diagnostic viable carcinoma.
Fourth row: Three cycles of nDoce treatment (3×) (case D2). (J) Low power view showing ulcerated skin surface at top with underlying necrosis (dashed arrow). Note adjacent TLS in lower right portion of image (solid arrow). (J) Low power view of CD45R-immunostained section showing dense population of B-cells in the TLS (solid arrow). (L) High power view of the necrotic area beneath the skin ulceration showing amorphous necrotic material with no diagnostic viable carcinoma cells.

### Histopathology:

Non-treated Control: On day of necropsy, the tumor volume in the non-treated control animal was measured and then tumor site tissues were dissected and approximately half the tumor was processed for docetaxel content and half was preserved for histological analysis. The non-treated control tumor contained an extensive diffuse proliferation of invasive carcinoma that measured up to 15 mm on the slides and consisted of sheets of tumor cells that were closely packed together (FIG. 29—Slide A). The tumor cells were large with pleomorphic nuclei that had vesicular chromatin and prominent eosinophilic nucleoli. The tumor cells had a moderate amount of lightly eosinophilic cytoplasm and they showed markedly increased mitotic activity (122 mitoses per 10 high power fields [400×hpf])(FIG. 29—Slide B). Individually necrotic and apoptotic tumor cells were present within the tumor and there were also scattered areas of coagulative tumor cell necrosis that overall occupied 5-10% of the tumor area. The foci of necrosis consisted of homogenous eosinophilic necrotic debris and this contained areas of admixed degenerating tumor cells (FIG. 29—Slide C). There was no significant lymphoid infiltrate within the tumor and in particular, there were no discrete small lymphoid collections or tertiary lymphoid structures (TLS) in the tumor tissue or in the surrounding non-neoplastic stromal tissue. The surrounding stroma contained a patchy mild immune cell infiltrate. Immunohistochemical staining for CD68 (marker of macrophages) highlighted a mild macrophage infiltrate within and around the tumor with increased density of staining within the foci of tumor necrosis, consistent with increased concentration of macrophages in areas containing increased cellular debris.

Non-treated Intratumoral vehicle group: On day of necropsy, tumor volumes in these IT vehicle animals were measured and then tumor site tissues were dissected and approximately half the tumor was processed for docetaxel content and half was preserved for histological analysis. The two intratumoral vehicle cases demonstrated similar findings at the morphologic and immunohistochemical level and both had a similar morphologic and immunohistochemical appearance to that seen in the above-mentioned control case. In particular, both cases contained extensive sheets of large carcinoma cells with an identical appearance to that seen in the control cases. The viable tumor measured up to 12 and 24 mm in maximum dimension on the slide in these two cases, respectively. Both cases also contained geographic areas of necrosis and this was fairly extensive in one case where it occupied>50% of the tumor area (case A3) (FIG. 29 Slide D). There was very limited non-neoplastic tissue for assessment in both cases although where present, this contained a mild immune cell infiltrate. There were no TLSs present.

Intravenous Docetaxel: On day of necropsy, tumor volumes in the IV docetaxel animals were measured and then tumor site tissues were dissected and approximately half the tumor was processed for docetaxel content and half was preserved for histological analysis. The two IV docetaxel cases demonstrated similar findings at the morphologic and immunohistochemical level and both had a similar morphologic and immunohistochemical appearance to that seen in the above-mentioned control case and the two IT vehicle cases. Specifically, both cases contained sheets of large viable carcinoma cells and interspersed areas of geographic tumor cell necrosis that occupied 11-50% (case B1) and 50-90% (case B3) of the tumor area in the two cases, respectively (see Table 12 below; FIG. 29—Slide E and FIG. 29—Slide F). Both cases had tumor masses that measured >10 mm in maximum dimension on the slide (11 mm and 15 mm) (see Table 9 below). The surrounding stromal tissue contained a mild immune cell infiltrate. There were no TLSs present.

Intratumoral nDoce 1 cycle: All three animals in this group contained residual carcinoma that was composed of similar pleomorphic cells as seen in the control, IT vehicle and IV docetaxel groups. However, the amount of residual carcinoma varied dramatically within this group. Specifically, two of the three cases (cases Cl and C6) contained extensive residual viable carcinoma that measured 16 mm and 19 mm in maximum dimension on the slide. These two cases also had geographic necrosis that occupied 11-50% of the tumor area. One of these two cases (case C1) contained a small amount of non-neoplastic tissue with a mild immune cell infiltrate. The other case did not have any non-neoplastic tissue present to assess for a surrounding immune cell infiltrate (Case C6). By contrast, the third case (case C4) showed necrosis of 50-90% of the tumor and in this case there was only a small focus of residual viable carcinoma present that measured 2.5 mm in maximum cross-sectional dimension on the slide (FIG. 30—Slide A and FIG. 30 Slide B). In this same case the surrounding non-neoplastic stroma contained a mild immune cell infiltrate (FIG. 30—Slide C). In addition, in the deeper immunohistochemical-stained sections a TLS was noted in the adjacent non-neoplastic fatty tissue. The TLS measured approximately 1 mm in maximum dimension and consisted of a dense, well-circumscribed collection of small mature lymphocytes showing organization into lymphoid follicles and a hilar region. Staining for CD45R confirmed that the majority of the lymphocytes in the TLS were B-cells and that these were organized into B-cell follicles within the TLS. As in the non-treated and vehicle controls, on day of necropsy, tumor volumes in these animals were measured and then tumor site tissues were dissected and approximately half the tumor was processed for docetaxel content and half was preserved for histological analysis.

Intratumoral nDoce 2 cycles: Four of the five animals in this group had the entirety of their tumor site tissue preserved for histological analysis. Two of the five animals (cases D2 and D8) in this group contained no residual viable carcinoma and these animals also demonstrated extensive geographic tumor necrosis (100% of tumor necrotic; FIG. 30—Slide H and FIG. 30—Slide I). In two of the remaining three animals (cases D4 and D6) there was also extensive necrosis (>90% of tumor) and in both cases there were only rare, tiny collections of detached tumor cells present, the largest of which measured up to 0.1 mm in each case. The significance of these rare tiny detached tumor cell clusters was not certain and given their appearance and detached localization adjacent to the edge of the tissue and edge of necrosis, an artifact of sectioning could not be excluded. In each of these four cases there was a single TLS. Three of the TLSs measured 1 mm, 1 mm and 2 mm, while the fourth measured 0.1 mm (case D8). The TLSs were discretely located within non-neoplastic tissue and were generally in the vicinity of, or directly adjacent to the necrotic material (FIG. 30—Slide D). The TLSs were well-circumscribed, but they lacked a fibrous capsule. The internal topology of the TLSs showed varying degrees of maturation but in the more mature-appearing TLSs there was a distinct resemblance to secondary lymphoid organs, with some of these having hilar regions with medullary sinuses that extended towards peripherally placed lymphoid follicles that were composed of homogenous small mature lymphocytes without visible nucleoli (FIG. 30—Slide F and FIG. 30—Slide G). The interfollicular areas also contained similar appearing small mature lymphocytes with occasional larger lymphoid cells consistent with immunoblasts. Focally, some of the lymphoid follicles contained germinal centers that were composed of a polymorphous lymphoid population that included small mature lymphocytes, intermediate-sized centrocytes and larger cells consistent with centroblasts (FIG. 30—Slide G). Occasional tangible-body macrophages were also noted in germinal centers. Immunohistochemical staining for CD45R showed strong staining of B-cells in the TLSs. Specifically, this result highlighted the B-cells in the lymphoid follicles, including germinal centers and showed absence of staining in the interfollicular lymphoid cells (T-cell areas)(FIG. 30—Slide E). The fifth case in this group (case D9) contained a residual focus of viable carcinoma that measured 8 mm in maximum dimension and also showed necrosis of 5-10% of the tumor area. This animal had approximately 50% of tumor site tissue preserved for histological analysis and 50% analyzed for docetaxel content. Staining for CD68 showed a moderate macrophage infiltrate in 1 of the 5 cases in this group (case D2) and a mild macrophage infiltrate in the remaining four cases (cases D4, D6, D8 and D9).

Intratumoral nDoce 3 cycles: None of the three animals (E1, E7, E9) in this group contained residual diagnostic viable invasive carcinoma nodules and all three cases also demonstrated extensive necrosis (FIG. 30—Slide L). All three animals in this group had the entirety of their tumor site tissues preserved for histological analysis. In two of these animals (E1 and E7) there was a large area of skin ulceration, subjacent to which was an area of necrosis that extended into surrounding non-neoplastic fibrofatty and muscular tissue. This was associated with regenerative changes in the surrounding epidermal lining that included areas of pseudoepitheliomatous hyperplasia, as well as degenerative changes in muscular cells. Similarly, within and adjacent to the necrosis there were regenerative larger stromal cells including fibroblasts and endothelial cells. There were also rare admixed single larger cells in the necrosis that had degenerating nuclei. These rare cells appeared to be in the process of necrosis or completely necrotic and while it was difficult to definitively exclude that these may have represented rare dying tumor cells, these could also have represented reactive/regenerative stromal cells or degenerating muscle cells as definitive muscle cells elsewhere in the section showed similar degenerative nuclear features. As such, the exact significance of these rare cells was not certain, but they did not form cohesive nodules and they appeared to be either dying or necrotic. A pancytokeratin (AE1/AE3) immunostain was performed to further assess these cells; however, while this showed lack of labeling of some of these larger cells, there was excessive background staining that made definitive assessment difficult in some areas. In addition, the pancytokeratin performed in this study overall was not reliable with lack of sensitivity in the control cases. As such, definitive assessment of these sections with the current keratin stain was not reliable and this will be deferred to review of slides stained with another keratin immunostain (keratin 7) which is currently pending. All three cases also contained a single, well-formed TLS and these measured 0.8 mm, 1.5 mm and 2 mm in maximum dimension in the three animals. The TLSs in this group (FIG. 30—Slide J and FIG. 30—Slide K) had a similar range of maturation and CD45R pattern of staining to that described in the nDoce 2 cycle group above. In particular, the TLS were well circumscribed and located in the vicinity of the necrosis and ulceration. The TLSs in this group showed internal organization with lymphoid follicles that were composed of B-cells that strongly expressed CD45R and some of these lymphoid follicles contained germinal centers. CD68 staining highlighted a moderate macrophage infiltrate in all three animals.

Tables 9 and 10 below reflect the maximum cross-sectional dimension of the viable carcinoma, as measured in millimeters on the slide.

TABLE 9
Maximum size of viable invasive carcinoma
on the slide in each group

| | | No | | | | |
|---|---|---|---|---|---|---|
| | # of | viable | <1 | 1-5 | 6-10 | >10 |
| Group | Animals | tumor | mm | mm | mm | mm |
| Control | 1 | | | | | 1* |
| IT vehicle 3 cycles | 2 | | | | | 2* |
| IV Docetaxel 3 cycles | 2 | | | | | 2* |
| IT nDoce 1 cycle | 3 | | | 1* | | 2* |
| IT nDoce 2 cycles | 5 | 2** | 2** | | 1* | |
| IT nDoce 3 cycles | 3 | 2** | | | | |

*On day of necropsy, approximately 50% of tumor site tissue was processed for analysis of docetaxel content and the remaining tumor site tissue was preserved for histological analysis.
**On day of necropsy the entirety of the tumor site tissue was preserved for histological analysis.

TABLE 10
Comparison of the non-nDoce treatment
groups with the IT nDoce groups

| | | No | | | | |
|---|---|---|---|---|---|---|
| | # of | viable | <1 | 1-5 | 6-10 | >10 |
| Group | Animals | tumor | mm | mm | mm | mm |
| non-nDoce-treated | 5 | | | | | 5* |
| IT nDoce-treated | 11 | 5** | 2** | 1* | 1* | 2* |

*On day of necropsy, approximately 50% of tumor site tissue was processed for analysis of docetaxel content and the remaining tumor site tissue was preserved for histological analysis.
**On day of necropsy the entirety of the tumor site tissue was preserved for histological analysis.

Table 9 shows the range of sizes of residual tumor in the six groups. Table 10 condenses this data to directly compare the size of the residual carcinoma nodules in the three non-nDoce groups (5 animals in total) with the three nDoce groups (11 animals in total). All five non-nDoce animals had residual viable carcinoma nodules that measured greater than 10 mm. By contrast, just under half (5/11) of the animals treated with IT nDoce had no diagnostic residual viable carcinoma on the slide to measure (complete regression). In two of the remaining 5 animals in the IT nDoce group that had residual viable carcinoma, this consisted of rare tiny tumor cell collections where tumor measured up to 0.1 mm in maximum dimension. The significance of the tiny amount of tumor in these cases was not certain as the detached localization and small size also raised the possibility of sectioning artifact. In a third case the residual tumor measured 2.5 mm and in the remaining three cases the tumors measured 8 mm, 16 mm and 19 mm in maximum dimension on the slide.

Comparison of the three IT nDoce groups with respect to percentage of cases with no residual invasive carcinoma and the size of residual viable carcinoma nodules on the slide is shown in Table 11.

TABLE 11
Comparison of tumor size in the three IT nDoce groups

| | | | Size of | % of cases |
|---|---|---|---|---|
| | | No | viable | with no |
| | # of | viable | nodules | residual |
| | Animals | tumor | (mm) | carcinoma |
| IT nDoce 1 cycle* | 3* | | 2.5, 16, 19 | 0% |
| IT nDoce 2 cycles | 5 | 2** | 0.1**, 0.1**, 8* | 40% |
| IT nDoce 3 cycles | 3** | 3 | N/A | 100% |

*On day of necropsy, approximately 50% of tumor site tissue was processed for analysis of docetaxel content and the remaining tumor site tissue was preserved for histological analysis.
**On day of necropsy the entirety of the tumor site tissue was preserved for histological analysis.

With progressive increase in the number of cycles of IT nDoce from 1 cycle to 3 cycles, the percentage of cases with no residual carcinoma increased. Specifically, the IT nDoce 1 cycle group had 0% (0/3) of cases with compete regression, although one of these cases measured only 2.5 mm, while the other two measured 16 and 19 mm on the slide. By contrast, the group given 2 cycles of nDoce had complete regression in 40% of cases (2/5). However, of the remaining three cases in this group that had residual viable carcinoma, this was extremely minimal, with clusters measuring up to 0.1 mm that could possibly have represented an artifact. Finally, the group given 3 cycles had complete regression in 100% (3/3) of the animals, with no residual viable carcinoma to measure in the any of the three cases in the IT nDoce 3 cycle group.

The percentage of tissue showing necrosis is shown in Table 12.

TABLE 12
Percentage of tumor showing necrosis

| | # of | | | 50- | 11- | 5- | |
|---|---|---|---|---|---|---|---|
| | Animals | 100% | >90% | 90% | 50% | 10% | <5% |
| Control | 1 | | | | | 1 | |
| IT vehicle 3 cycles | 2 | | | 1 | 1 | | |
| IV Docetaxel 3 | 2 | | | 1 | 1 | | |
| cycles | | | | | | | |
| IT nDoce 1 cycle | 3 | | | 1 | 2 | | |
| IT nDoce 2 cycles | 5 | 2 | 2 | | | 1 | |
| IT nDoce 3 cycles | 3 | 3 | | | | | |

All 16 animals in this study contained geographic tumor cell necrosis and in the non-nDoce-treated cases this included two cases with 50-90% tumor necrosis. However, overall the extent of tumor cell necrosis was significantly greater in the nDoce-treated group than in the non-nDoce-treated group. Specifically, 5 of the 11 nDoce-treated animals showed 100% tumor cell necrosis (complete regression) and 2 of the remaining 6 animals showed >90% tumor cell regression. By contrast, none of the 5 non-nDoce-treated animals showed >90% tumor cell necrosis.

The macrophage infiltrate density in surrounding non-neoplastic tissue based on assessment of H&E and immunohistochemical staining with CD68, graded semi quantitatively is shown in Table 13.

TABLE 13
Macrophage infiltrate density per treatment group

| | # | Mild | Moderate | Marked |
|---|---|---|---|---|
| Control | 1 | 1 | | |
| IT vehicle 3 cycles | 2 | 2 | | |
| IV Docetaxel 3 cycles | 2 | 2 | | |
| IT nDoce 1 cycle | 3* | 2 | | |
| IT nDoce 2 cycles | 5 | 4 | 1 | |
| IT nDoce 3 cycles | 3 | | 3 | |

The intensity of the macrophage infiltrate in the surrounding non-neoplastic tissue in all animals was not striking; however, when the non-nDoce-treated group was compared to the nDoce-treated group, it was noted that the latter contained cases with a moderate degree of macrophage infiltrate while this was not seen in the non-nDoce-treated group. * One case in the IT nDoce-treated 1 cycle group did not contain surrounding non-neoplastic tissue for assessment.

The number of cases in each group that contained at least one TLS is shown in Table 14.

TABLE 14
Number of cases with TLSs in each group

| | # of | # containing at |
|---|---|---|
| | Animals | least one TLS |
| Control | 1 | 0 |
| IT Vehicle 3 cycles | 2 | 0 |
| IV Docetaxel 3 cycles | 2 | 0 |
| IT nDoce 1 cycle | 3 | 1 |
| IT nDoce 2 cycles | 5 | 4 |
| IT nDoce 3 cycles | 3 | 3 |

None of the 5 cases in the non-nDoce-treated group contained TLSs. However, 8 of the 11 animals in the nDoce-treated group contained a TLS and in all but one of these 8 cases, the TLS measured at least 1 mm in maximum dimension. Of particular importance, the presence or absence of a TLS was closely linked with the presence or absence of residual carcinoma. Specifically, all cases that had either no diagnostic residual carcinoma (5 cases) or residual carcinoma that measured 2.5 mm or less (3 cases) also contained a TLS and these were the only cases that contained a TLS. By contrast, none of the remaining cases, all of which had residual carcinoma measuring at least 8 mm on the slide, contained a TLS.

The comparison of necropsy volume to maximum tumor size as measured on the slide is shown in Table 15.

TABLE 15
Comparison of Necropsy volume to maximum
tumor size as measured on the slide

| | | |
|---|---|---|
| | Necropsy | Maximum tumor size |
| Group | volume (mm³) | on slide (mm) |

Control

| | | |
|---|---|---|
| F1: | N/A | 15 |

IT Vehicle 3 cycles

| | | |
|---|---|---|
| A3: | 3497 | 12 |
| A8: | 3781 | 24 |

IV Docetaxel 3 cycles

| | | |
|---|---|---|
| B1: | 2872 | 15 |
| B3: | 1652 | 11 |

IT nDoce 1 cycle

| | | |
|---|---|---|
| C1: | 1458 | 19 |
| C4: | 323 | 2.5 |
| C6: | 1780 | 16 |

IT nDoce 2 cycles

| | | |
|---|---|---|
| D2: | 22 | 0 |
| D4: | 13 | 0.1 |
| D6: | 59 | 0.1 |
| D8: | 14 | 0 |
| D9: | 392 | 8 |

IT nDoce 3 cycles

| | | |
|---|---|---|
| E1: | 50 | 0 |
| E7: | 101 | 0 |
| E9: | 0 | 0 |

When the tumor-site volume at necropsy was compared to the maximum carcinoma length on the slide, the trend seen in the tumor length on the slide amongst the different treatment groups was also seen in the necropsy tumor volume, supporting that the tumor measurement on the slide was a representative assessment of the different responses to treatment in the different animals (see Table 15). In animals where a tiny volume of tumor site was recorded at necropsy and no carcinoma, or very minimal carcinoma, was seen on microscopic examination, the small volume noted at necropsy may have been predominantly or entirely due to necrotic or fibrotic tissue. Alternatively, a 1-2 mm TLS could also have been detected in the tumor site at the time of necropsy and its measurement may have contributed to some of the recorded tumor-site volumes.

### Discussion:

The morphologic and immunohistochemical features of a subset of 16 mice from the bladder carcinoma study aimed to assess the general safety and efficacy of intratumoral nDoce. The current subset of 16 animals included 1 non-treated control animal, 2 animals given intratumoral vehicle, 2 animals treated with intravenous docetaxel (3 cycles) and 11 animals treated with intratumoral nDoce. The nDoce group was separated into 3 groups based on the number of administered cycles: group 1 (1 cycle. 3 animals); group 2 (2 cycles. 5 animals); and group 3 (3 cycles. 3 animals).

The two main features that differed amongst the various groups were the presence and degree of tumor regression and the presence of tertiary lymphoid aggregates. In particular, there was prominent tumor regression in the majority of the animals in the intratumoral nDoce groups while there was no overt tumor regression in any of the animals in the other groups. Mirroring this finding, all the animals in the nDoce group with significant regression contained a TLS, whereas none of animals that had persistent tumors without overt regression contained a TLS.

In this microscopic review, the residual viable carcinoma maximum dimension on the slide was used to compare the degree of response in the different groups. The corresponding maximum tumor length at necropsy was not available for comparison; however, the tumor volume at necropsy was available. When the tumor volume at necropsy was compared to the tumor length on the slide, the trend seen in the tumor length on the slide amongst the different treatment groups was also seen in the necropsy tumor volume, supporting that the tumor measurement on the slide was a representative metric to use in order to compare the different responses to treatment in the different animals (Table 15). In the non-nDoce group, all five animals contained extensive residual viable carcinoma that measured at least 11 mm in maximum dimension on the slide (range: 11 mm-24 mm). By contrast, just under half (5/11) of the animals treated with IT nDoce had no diagnostic residual viable carcinoma on the slide to measure (complete regression). In two of the remaining 5 animals in the IT nDoce group that had residual viable carcinoma, this consisted of rare tiny tumor cell collections where tumor measured up to 0.1 mm in maximum dimension. The significance of the tiny amount of tumor in both of these cases was not certain as the detached localization and small size also raised the possibility of sectioning artifact resulting in a false positive finding in these cases. In a third case the residual tumor measured 2.5 mm and in the remaining three cases the tumors measured 8 mm, 16 mm and 19 mm in maximum dimension on the slide (Tables 9 and 10).

All 16 animals in this study contained areas of geographic tumor cell necrosis that represented at least 5% of the tumor area. However, when all cases were taken together in both groups, the extent of tumor cell necrosis was significantly greater in the nDoce group than in the non-nDoce group. Specifically, 5 of the 11 nDoce animals showed 100% tumor cell necrosis (complete regression) and 2 of the remaining 6 animals in this group showed >90% tumor cell regression. By contrast, none of the 5 non-nDoce animals showed >90% tumor cell necrosis. Specifically, in non-nDoce group, 3 of the 5 cases had less than 50% necrosis while 2 of the 5 cases in the non-nDoce cases showed 50-90% tumor necrosis (Table 12).

When the three nDoce groups (1 cycle, 2 cycles, 3 cycles) were compared together, it was noted that a progressive increase in the number of cycles of IT nDoce from 1 cycle to 3 cycles, was associated with an increase in the percentage of cases that had no residual carcinoma. Specifically, the IT nDoce 1 cycle group had 0% (0/3) of cases with compete regression, although in one of these cases the residual viable carcinoma nodule measured only 2.5 mm on the slide, while the other two cases had residual viable carcinoma that measured 16 and 19 mm on the slide. By contrast, the group given 2 cycles had complete regression in 2 of 5 cases (40%). In addition, in two of the remaining three cases in this group that had residual viable carcinoma, the size of the residual carcinoma was extremely minimal, with clusters measuring up to 0.1 mm in maximum dimension. Given the peripheral and detached localization of the tiny clusters in these two animals, these could possibly have represented an artifact of sectioning resulting in a false positive in these two animals, in which case the actual complete regression rate would have been 4/5 (80%) in the group given 2 cycles. The last animal in the 2 cycle group had residual carcinoma measuring 8 mm. Finally, the group given 3 cycles of nDoce had complete regression in 100% (3/3) of the animals, with no residual viable carcinoma available to measure in the any of the three cases in the IT nDoce 3 cycle group (Table 11).

Another striking finding in this study was the presence of tertiary lymphoid structures (TLSs) in all of the nDoce animals that demonstrated a significant response to treatment. Specifically, a TLS was found in 8 animals and all of these were in the nDoce group. These 8 animals that contained a TLS included the 5 animals with no residual viable carcinoma; the two animals with rare detached clusters of carcinoma measuring up to 0.1 mm; and the animal with a residual carcinoma focus measuring 2.5 mm. None of the remaining animals, all of which had residual carcinoma nodules measuring at least 8 mm, had any TLSs. This finding demonstrated a very strong correlation between the presence of a TLS and a significant tumor response to therapy. In addition, a TLS was only seen in animals that received IT nDoce and within that group, a TLS was present in 8 of the 11 animals, including all three animals given 3 cycles of nDoce.

The TLSs in this study ranged in size from 0.1 up to 2 mm; however, 7 of the 8 TLSs were at least 1 mm in maximum dimension and two measured up to 2 mm. Given these sizes, the TLSs in most of these animals were easily appreciated by naked eye examination of the stained slides as a discrete nodule and in turn these may have been palpable in the in vivo state. All of the TLSs were well circumscribed, and they lacked a well-formed capsule. They showed varying stages of maturation with the most mature TLSs having well-formed peripheral lymphoid follicles composed of mature B-cells that labeled strongly with CD45R and intervening interfollicular “T-cell areas” as well as medullary areas with sinuses. Some of the TLSs showed evidence of activation with lymphoid follicles containing germinal centers.

Finally, there was an associated macrophage infiltrate in the non-neoplastic tissue that generally correlated with the degree of tumor response to therapy. In particular, all of the animals in the non-nDoce group had a mild macrophage infiltrate while the nDoce group included cases with a mild and a moderate immune cell infiltrate. All four cases with a moderate immune cell infiltrate had complete tumor regression and this included all three animals in the group given 3 cycles of IT nDoce.

### Conclusions:

In conclusion, this study performed on a subset of 16 mice from the bladder carcinoma cohort clearly showed a strong association between IT nDoce therapy and tumor regression with 5 of 11 animals treated with IT nDoce showing complete tumor regression while a further 3 animals in this group had minimal residual tumor that measured 0.1 mm, 0.1 mm and 2.5 mm in maximum extent. Moreover, increasing cycles of IT nDoce (moving from 1 cycle to 3 cycles) resulted in a greater degree of tumor regression with all three animals in the 3-cycle group showing complete tumor regression. Furthermore, a tertiary lymphoid structure (TLS) was seen in all 8 animals that demonstrated a significant tumor response while a TLS was not seen in any of the animals that did not show a significant tumor response. These findings suggest that in animals given IT nDoce there is significant interplay between the local drug effect on the tumor and the host animal's immune system that results in formation of a robust local TLS adjacent to the tumor that in turn sets up a rapid feedback loop of adaptive and humoral immunity which further contributes to the significant tumor regression.

### Example 5. In-Vitro Release Testing Study. Comparative Measurements of Paclitaxel and Docetaxel Concentration Equilibration Across Natural Membranes

An in-vitro release testing study was conducted to comparatively measure the flux of formulations of various forms of paclitaxel and docetaxel across natural epithelial membranes.

Test Articles:

Paclitaxel particles (nanoparticulate paclitaxel powder, approximately 98% paclitaxel with a mean particle size (number) of 0.827 microns, a SSA of 27.9 mg²/g, and a bulk density (not tapped) of 0.0805 g/cm³ used in this example) in Suspension, 6 mg/mL.
Docetaxel particles (nanoparticulate docetaxel powder, approximately 99.5% docetaxel with a mean particle size (number) of 0.915 microns, a SSA of 33.4 mg²/g, and a bulk density (not tapped) of 0.0675 g/cm³ used in this example) in Suspension, 10 mg/mL.
Abraxane® diluted to 6 mg/mL.
Paclitaxel solution for injection diluted to 6 mg/mL.
Docetaxel solution for injection diluted to 10 mg/mL.

Epithelial membrane substrates: Porcine bladder and porcine intestine were sourced. Upon receipt of the bladder and intestine, the membranes were stored at −20° C. until used. Prior to use, the membranes were removed from the freezer and allowed to thaw fully at ambient temperature.

Equipment:

Franz-type Diffusion Cells (FDCs): 64 diffusion cells with 3.3 ml receptor volume and a 0.55 cm2 receptor fluid exposure surface area.
Stirring Dry Block Heaters: Reacti-Therm #18823 stirring dry block heaters were used to maintain the receptor fluid at 32±0.5° C. with constant stirring throughout the study.
Agilent 1260 HPLC unit with a G16120 MS detector, ID #: TM-EQ-069.

Receptor Fluid: The receptor fluid consisted of 60 vol %/40 vol % methanol/water at pH 4 with 0.01 wt % NaN₃ (added as a preservative). The solubility of paclitaxel and docetaxel in the Receptor Fluid was determined to be sufficient to maintain sink conditions throughout the study. After mixing and degassing the Receptor Fluid it was filtered through a ZapCap CR 0.2 μm membrane under vacuum; the Receptor Fluid, so filtered, was stirred for an additional 20 minutes under vacuum.

Experimental Procedure:

1. The receptor wells were filled with degassed Receptor Fluid using a pipette.
2. A 6 mm by 3 mm diameter Teflon coated magnetic stir bar was introduced into each receptor well.
3. The defrosted and washed bladder or intestine pieces were examined and only areas of even thickness and with no visible surface damage were used.
4. The bladder and intestine pieces were cut into approximately 2 cm×2 cm squares using skin scissors. The square sizes were adjusted as necessary according to the shape and dimensions of the substrate, but were selected to be approximately uniform in size among all FDCs.
5. Substrate pieces were centered on each inverted donor compartment.
6. The donor and receptor well compartments were then aligned and clamped together with a pinch clamp, ensuring that the substrate pieces were centered between both donor and receptor wells.
7. Additional Receptor Fluid was added as necessary. Air bubbles in the receptor well, if any, were removed by tilting the FDC assembly such that the air escapes along the sample port. Receptor wells were filled with approximately 3.3 ml of Receptor Fluid.
8. The assembled FDCs were placed into stirring dry block heaters which were preheated to 32° C. The Receptor Fluid was continuously agitated via the magnetic stir bar.
9. After 20 minutes, the surface of the membranes in each FDC was examined. If the membranes appeared wet or showed signs of being compromised, the cell was discarded.

Test Article Application Procedure: After the membrane integrity tests were complete and the cells appropriately sorted, samples of the test articles were then applied to the surface of the substrate. A one-time dosing regimen was used for this study. For all formulations, 100 μl of the formulation was introduced into the donor cells. The donor cells were then capped for the remainder of the experiment. For the paclitaxel particles suspension, Paclitaxel solution for injection, and Abraxane®, the amount of paclitaxel drug active corresponded to 0.6 wt % correlating to a dose of 1091 μg/cm². For the docetaxel particles suspension and docetaxel solution for injection, the amount of docetaxel drug active corresponded to 1.0 wt % correlating to a dose of 1818 μg/cm². “Blank” undosed FDC cells were also set up to test for background signal noise. The background noise measured from these “blank” cells was negligible.

Sampling of Receptor Fluid: Using a graduated Hamilton type injector syringe, a 300 μl aliquot was abstracted from the sampling port of each FDC at each of 1, 3, 8, and 24 and 47 hours. Fresh Receptor Fluid was added to each receptor well to replace the volume of fluid abstracted. Each abstracted aliquot was introduced into a well in a 96-well microtiter plate. Samples were stored in a refrigerator at 4-8° C. prior to MS analysis. Samples were analyzed within 5 days of collection.

Analysis of Sample: The samples abstracted from the receptor wells were then analyzed using a MS method. The concentrations of the Active were assayed and reported in each case. After the MS testing was complete, the samples were analyzed using Chemstation software. The AUCs of the paclitaxel or docetaxel peaks were recorded and converted to μg/ml values using a calibration curve developed from the calibration standards' AUC values and known concentration values. These μg/ml values were imported into the study results Excel workbook. These concentrations were then multiplied by the receptor volume (3.3 mL) and divided by the surface area of the skin exposed to the receptor fluid (0.55 cm²) for an end cumulative amount in μg/cm². For receptor fluid time points greater than 1 hr, this μg/cm² value was corrected for the sample aliquot volumes which were removed to compensate for the dilution caused by replacing the sample volume with fresh buffer solution. As an example, for the second time point at 3 hrs, the dilution factor (300 μl aliquot/3.3 ml receptor volume or 1/11) is multiplied by the μg/cm² value calculated for the 1 hr time point, the result of which is then added to the μg/cm² concentration which is calculated using the 3 hr AUC value.

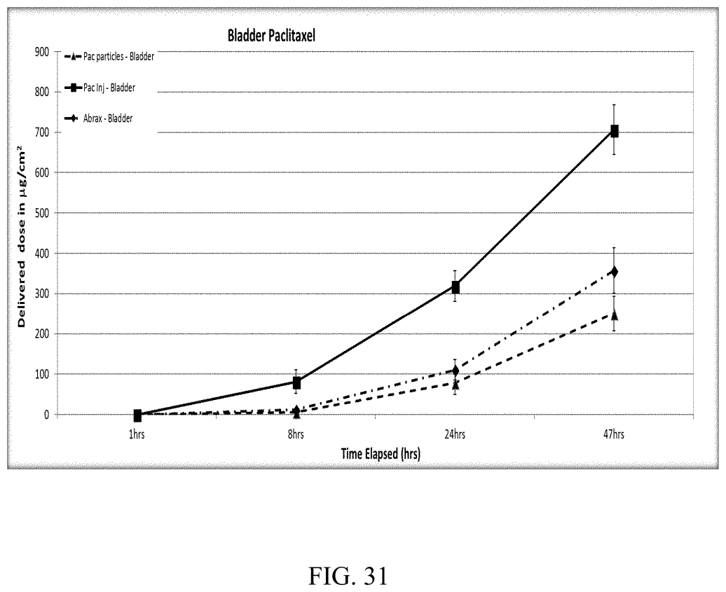

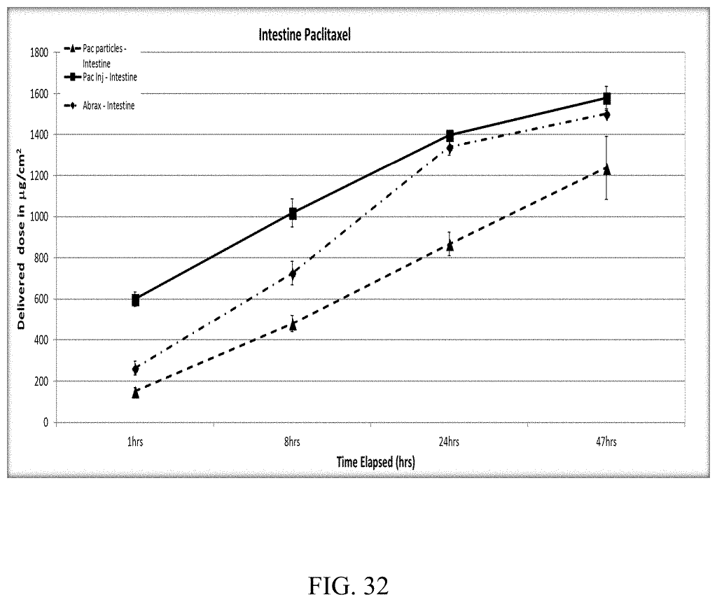

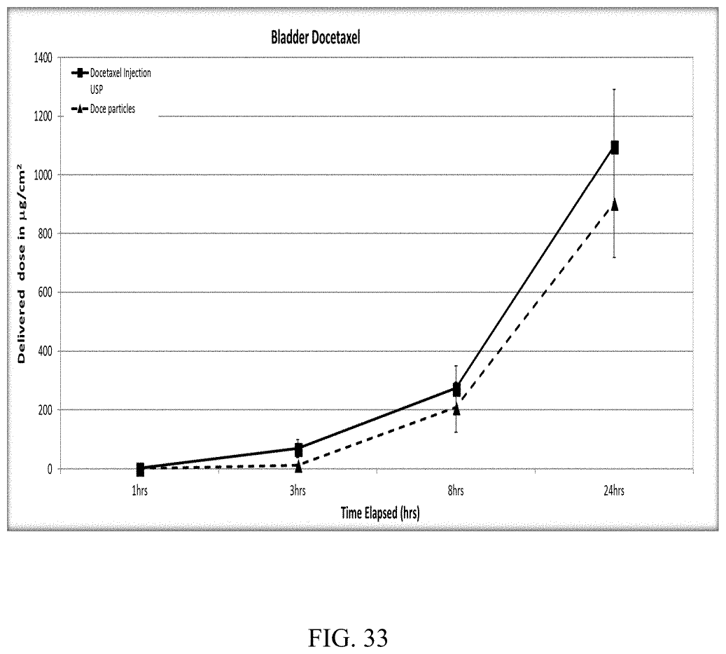

Results: The results are shown in FIG. 31, FIG. 32, and FIG. 33.

FIG. 31 is a graph of the flux of paclitaxel (delivered dose of paclitaxel active drug across a porcine bladder membrane over time) from various paclitaxel formulations.

FIG. 32 is a graph of the flux of paclitaxel (delivered dose of paclitaxel active drug across a porcine intestinal membrane over time) from various paclitaxel formulations. Note: flux amounts greater than dose amounts were attributable to evaporation of the receptor fluid.

FIG. 33 is a graph of the flux of docetaxel (delivered dose of docetaxel active drug across a porcine bladder membrane over time) from various docetaxel formulations. Note: the 48-hour timepoint was discarded due to evaporation issues with the receptor fluid samples.

As can be seen in the figures, the paclitaxel particles and docetaxel particles suspensions had the lowest flux across the membranes as indicated by the least amount of active drug delivered over time through the membranes. These results indicate that the paclitaxel particles are retained on one side of an epithelial membrane in greater amounts than Abraxane® or paclitaxel solution over time. Also, the docetaxel particles are retained on one side of an epithelial membrane in greater amounts than docetaxel solution over time. This would suggest that paclitaxel particle suspension when injected into an epithelial cyst would reside within the cyst in greater amounts over time than would Abraxane® or paclitaxel solution, and that docetaxel particles suspension when injected into an epithelial cyst would reside in the cyst in greater amounts over time than would docetaxel solution.

### Example 6. Phase 1/2 Trial Evaluating the Safety and Tolerability of Docetaxel Particles Suspension in Injection into Tumor Resection Site and Intravesical Instillation in Subjects with Urothelial Carcinoma

This Phase 1/2 study will include subjects with non-muscle invasive bladder cancer (NMIBC) and muscle invasive bladder cancer (AMC). As disclosed herein, the nDoce is an aqueous suspension of docetaxel particles of at least 95% docetaxel, with a mean particle size (number) of 0.1 microns to 5 microns, an SSA of at least 18 m²/g, and a bulk density (not tapped) of 0.05 g/cm³ to 0.15 g/cm³ to be used in this example.

Objectives: The primary objective of this study is to evaluate the safety and tolerability of nDoce injected directly into the bladder wall tumor resection site after surgical resection and instilled intravesically. Secondary objectives are (a) to characterize the pharmacokinetics (PK) of nDoce when injected directly into the bladder wall tumor resection site in the presence of intravesical instillation; and (b) to determine whether any of the nDoce concentrations (0.75, 1.5, 2.5, or 3.75 mg/mL administered by injection; 2.0 or 3.0 mg/mL administered by intravesical instillation) show signs of preliminary efficacy.

Description of Study: This open-label Phase 1/2 study will enroll subjects with pathological or cytological diagnosis of high-risk NMIBC or MIBC. Subjects will be stratified into two treatment groups, Group 1 (NMIBC) and Group 2 (MIBC). The study drug will be delivered by direct injection into the bladder wall tumor resection site and by intravesical instillation. At Visit 2, all subjects will receive nDoce injected into the index tumor resection site on the bladder wall, immediately following TURBT, followed by an initial nDoce intravesical instillation (within 2 hours of the direct injection).

Group 1 (NMIBC): After a recovery period, Group 1 subjects will proceed to the 3-month Induction period. Subjects will be assessed for recovery (TURBT resection site healing) at 4 weeks (1 month) after Visit 2, at which point the Investigator will evaluate subject symptoms, pathology (if available), gross hematuria or urinalysis findings. If the Investigator determines that the subject has not recovered at 4 weeks, evaluations will be repeated at least every 2 weeks until the subject has recovered and intravesical nDoce can be administered.

The 3-month Induction period consists of 6 weekly nDoce intravesical instillations, followed by 6 weeks of rest. After the Induction period, following confirmation of non-recurrence, subjects will proceed to a 3-month Maintenance period, consisting of 3 weekly nDoce intravesical instillations, followed by 9 weeks of rest. Subjects will return at Month 6 for an End of Treatment study visit. Plasma samples will be collected at Visit 2 (prior to nDoce injection and at 1, 2, 4, 6, and 24 hours post-injection), at Visit 3 (prior to nDoce intravesical instillation and at 1, 2, 4, 6, and 24 hours post-intravesical instillation), at Visits 4-11 prior to the nDoce intravesical instillation, and at End of Treatment to characterize the PK of nDoce.

Subjects will be evaluated for tumor recurrence with cystoscopy and urine cytology at Visit 9, End of Treatment, or at any time, at the discretion of the Investigator. Biopsy is to be performed at any time at the discretion of the Investigator for positive or suspicious cytology or cystoscopic findings. Additionally, subjects will be followed 30 days after the last administration of study drug for safety, and tumor response to therapy. Institution pathology and immuno-histochemical reports will be collected for any resection, cystectomy or biopsy specimen to include, but not limited to, bladder resection, biopsies performed at any time during the study or early withdrawal cystectomy tissue or node samples.

Group 2 (MIBC): At the end of Visit 2, Group 2 (MIBC) subjects will proceed to institutional standard of care (SOC) treatments and return for the End of Treatment study visit 30 days (+/−5 days) after Visit 2. Plasma samples will be collected at Visit 2 (prior to nDoce injection, at 1, 2, 4, 6, and 24 hours post-injection) and at the End of Study visit to characterize the PK of nDoce.

Groups 1 and 2: The study will consist of a dose escalation phase and a dose confirmation phase for the direct injection of nDoce concentrations (0.75, 1.5, 2.5, or 3.75 mg/mL) for Groups 1 and 2. In the direct injection dose escalation phase, subjects will be enrolled in sequential cohorts of three subjects starting at the lowest concentration. Following Data Safety Monitoring Board (DSMB) review of the cohort data, with the exception of the PK data, the DSMB will determine whether to: (a) escalate to the next dose level cohort (no DLT (dose-limiting toxicity)); (b) add three additional subjects to the current cohort (one DLT); (c) if still at the first cohort, stop the study (2 or more DLT); (d) if at higher cohorts return to the previous (lower) dose cohort and expand by three subjects (more than one DLT). The dose determined to be most suitable for further evaluation, defined as the highest dose with an acceptable safety and tolerability profile (as determined by the DSMB) will enroll additional subjects to total up to 12 subjects at that direct injection dose level.

The study will also dose escalate for Groups 1 and 2 for the intravesical instillation of nDoce concentrations (2.0 and 3.0 mg/mL). In the intravesical instillation dose escalation phase, the first three subjects in Groups 1 and 2 will be enrolled at the lowest concentration of 2.0 mg/mL for the Visit 2 instillation. If the dose is well-tolerated, the first three subjects will receive subsequent Induction and Maintenance intravesical instillations at 3.0 mg/mL and future cohorts will receive all intravesical instillations at 3.0 mg/mL.

Primary Endpoint: The primary endpoint will be safety and tolerability as demonstrated by AE, changes in laboratory assessments, physical examination findings and vital signs.

Secondary Endpoints: The secondary endpoints will be: Concentration of docetaxel in the systemic circulation post-injection in the presence of intravesical instillation (as determined by PK analysis); and tumor recurrence.

Study Drug: The study drug will be supplied in clinical supplies kit. Each kit will contain one vial of nDoce particles and one vial of Sterile Reconstitution Solution. The nDoce powder vial will contain sterile nanoparticulate docetaxel particles at 100 mg/vial appearing as a white powder. The sterile reconstitution solution will contain 1% Polysorbate 80, NF and 8% Ethanol, USP in normal saline solution (0.9% Sodium Chloride for Injection, USP). When ready for use, the nDoce particles will be suspended in the Sterile Reconstitution Solution.

Preparation of Study Drug: Direct Injection: An appropriate amount of the reconstitution solution will be added to the nDoce powder vial to reconstitute the drug in suspension to the required cohort-assigned dose for injection (3.0, 6.0, 10.0 or 15.0 mg). Once the drug has been reconstituted, just over the maximum injection volume of 4.0 mL of the suspension will be withdrawn from the vial into a syringe. Intravesical Instillation: An appropriate amount of the reconstitution solution will be added to the nDoce powder vial to reconstitute the drug in suspension to 2.0 mg/mL or 3.0 mg/mL. Once the drug has been reconstituted, the appropriate volume of the suspension will be withdrawn from the vial and transferred to the saline bag (0.9% Sodium Chloride for Injection USP) for instillation.

Dosing and Administration:

Groups 1 and 2: Direct Injection: Subjects will receive the assigned nDoce injection dose into the base of the index tumor resection site on Visit 2 immediately post-TURBT. The index tumor resection site is defined as the largest resection site (should not exceed 8.1 cm²) if multiple resections are performed. If multiple resections are performed, only the index tumor resection site will receive nDoce injections. Adjustable tip-length cystoscopy needles are to be used for injection into the resected bladder wall. The needle tip to be adjusted to 2 mm (per manufacturer recommendation) for injections in the dome area of the bladder and 3-4 mm for injections in the side area of the bladder. A total volume of 4.0 mL of nDoce will be injected in 0.5 mL increments, approximately 1 cm apart, with up to 8 injections into the index tumor resection site including up to 5 mm outside the resection margin. Injections will be performed in a tangential approach (grid-like pattern to cover the resection site) so the needle tip is viewable under direct visualization by cystoscope. A 70°-degree diagonal cystoscope view may be used. The total dose administered will not exceed the assigned cohort dose of 3.0 mg (0.75 mg/mL), 6.0 mg (1.5 mg/mL), 10.0 mg (2.5 mg/mL), or 15.0 mg (3.75 mg/mL).

Groups 1 and 2: Visit 2 Intravesical Instillation: The initial intravesical instillation will immediately follow the nDoce direct injection (≤2 hours). nDoce will be instilled intravesically in the bladder for a maximum of 30 minutes (+/−5 min). The total dose administered will not exceed the assigned cohort dose 50 mg in 25 mL of saline for a final concentration of 2.0 mg/mL or 75 mg in 25 mL of saline for a final concentration of 3.0 mg/mL. The subject will be in supine position. Local anesthetic gel is allowed for catheter placement. The urinary catheter will be inserted into the bladder using sterile technique. Isotonic saline or sterile water is the only distending medium which will be allowed in this study. Following intravesical instillation, the subject will be asked to change position every 15 minutes to ensure uniform coating of study medication to the bladder wall. On Visit 2, at the end of the 30-minute dwell time, the instillate will be drained by catheter into an appropriate receptacle; the catheter will be removed, and the drained fluid and catheter will be disposed of.

Group 1 Only: Induction and Maintenance Intravesical Instillation: Visits 3-8 Induction: nDoce will be instilled in the bladder once/week for 6 weeks for a maximum of 120 minutes (+/−10 min). Visits 9-11 Maintenance: nDoce will be instilled in the bladder once/week for 3 weeks for a maximum of 120 minutes (+/−10 min).

Starting Dose and Dose Escalation Schedule:

Direct Injection: The study will consist of a dose escalation phase and a dose confirmation phase for the direct injection of nDoce concentrations (0.75, 1.5, 2.5, or 3.75 mg/mL) for Groups 1 and 2 as shown in Table 16.

TABLE 16
Direct Injection Dose Escalation

| | | | Total Suspension |
|---|---|---|---|
| | Suspension | Max # of | Administered Not |
| Cohort | Concentration | Injections | to Exceed |
| 1 | 0.75 mg/mL | 8 | 3.0 mg |
| 2 | 1.5 mg/mL | 8 | 6.0 mg |
| 3 | 2.5 mg/mL | 8 | 10.0 mg |
| 4 | 3.75 mg/mL | 8 | 15.0 mg |

During direct injection dose escalation, cohorts will be enrolled sequentially starting at the lowest dose (3.0 mg). Cohorts will enroll separately for Groups 1 and 2. Each cohort will have a planned minimum of three subjects for Group 1 and three subjects for Group 2. Escalation to the next cohort in each group will proceed, independent of the other group, following review of data. All clinical data from subjects in each cohort, including all DLTs and excluding PK, will be reviewed and evaluated once all three subjects have completed Visit 3, to determine if the dose received is considered safe and tolerable, and to determine if dose escalation may occur. At the initial review, if cohort 1 (3.0 mg) is determined safe (no DLT), escalation to the next dose level, cohort 2 (6.0 mg), will occur. If >1 DLT occurs at cohort 1, three additional subjects will be added to cohort 1. If >1 DLT occurs in the additional three subjects, the study will stop. If no additional DLT occurs in the additional three subjects, the study will escalate to the next dose level, cohort 2 (6.0 mg). Three subjects will be dosed at cohort 2 (6.0 mg). If it is determined cohort 2 is safe (no DLT), escalation to the next dose level, cohort 3 (10.0 mg), will occur. If >1 DLT occurs at cohort 2, three additional subjects will be added to cohort 2. If >1 DLT occurs in the three additional cohort 2 subjects, the study will return to the previous (lower) dose, cohort 1 (3.0 mg), and proceed to dose confirmation. If no additional DLT occurs in the additional three subjects, the study will escalate to the next dose level, cohort 3 (10.0 mg). Three subjects will be dosed at cohort 3 (10.0 mg). If it is determined cohort 3 is safe (no DLT), escalation to the next dose level, cohort 4 (15.0 mg), will occur. If >1 DLT occurs at cohort 3, three additional subjects will be added to cohort 3. If >1 DLT occurs in the three additional cohort 3 subjects, the study will return to the previous (lower) dose, cohort 2 (6.0 mg), and proceed to dose confirmation. If no additional DLT occurs in the additional three subjects, the study will escalate to the next dose level, cohort 4 (15.0 mg). Three subjects will be dosed at cohort 4 (15.0 mg). If it is determined cohort 4 is safe (no DLT), dose confirmation at cohort 4 (15.0 mg) will occur. If >1 DLT occurs at cohort 4, three additional subjects will be added to cohort 4. If >1 DLT occurs in the three additional cohort 4 subjects, the study will return to the previous (lower) dose, cohort 3 (10.0 mg), and proceed to dose confirmation. If no additional DLT occurs in the additional three subjects, the study will complete enrollment at cohort 4 (15.0 mg) in dose confirmation. The dose most suitable for further evaluation will be the highest dose with an acceptable safety and tolerability profile. If one or fewer subjects in a six-subject cohort, or no subjects in a three-subject cohort at the highest dose, experience DLT, that cohort will be taken into the dose confirmation phase. If greater than one subject in a six-subject cohort experience DLT, the previous dose will be taken into the dose confirmation phase. Once the dose deemed appropriate for further evaluation has been determined, additional subjects will be enrolled to provide up to a total of 12 subjects dosed at that dose level.

Intravesical Instillation: The study will also dose escalate for Groups 1 and 2 for the intravesical instillation of nDoce concentrations (2.0 and 3.0 mg/mL). In the intravesical instillation dose escalation phase, the first three subjects in Groups 1 and 2 will be enrolled at the lowest concentration of 2.0 mg/mL for the Visit 2 instillation. Escalation to 3.0 mg/mL in each group will proceed, independent of the other group, following review of data. If no subjects in cohort 1 at 2.0 mg/mL experience a DLT, the intravesical dose will escalate to 3.0 mg/mL for that group as described below. If two or more subjects in cohort 1 experience a DLT at 2.0 mg/mL, then the study will stop. If one of the three subjects in cohort 1 experiences a DLT at 2.0 mg/mL, then an additional three subjects will be enrolled to cohort 1 at 2.0 mg/mL. If, in the additional three subjects at 2.0 mg/mL, no subjects experience a DLT, then the dose will remain at 2.0 mg/mL as described below. If, in the additional three subjects at 2.0 mg/mL, one more subjects experience a DLT, then the study will stop. Group 1: All clinical data from subjects in cohort 1, including all DLTs described in this section and excluding PK, will be reviewed and evaluated once all three subjects in cohort 1 have completed Visit 3, to determine if 2.0 mg/mL is considered safe and tolerable, and to determine if dose escalation may occur. If 2.0 mg/mL is well tolerated, as described above, cohort 1 will receive 3.0 mg/mL for all subsequent Induction and Maintenance instillation visits and future subjects in Group 1 will receive 3.0 mg/mL for all intravesical instillations. Group 2: All clinical data from subjects in cohort 1, including all DLTs described in this section and excluding PK, will be reviewed and evaluated once all three subjects in cohort 1 have completed the End of Treatment visit, to determine if 2.0 mg/mL is considered safe and tolerable, and to determine if dose escalation may occur. If 2.0 mg/mL is determined to be well tolerated at 2.0 mg/mL, future subjects in Group 1 will receive 3.0 mg/mL for all intravesical instillations.

Definition of Dose Limiting Toxicity (DLT): Included in the review of AEs (adverse events) and general study data pertaining to safety (laboratory results, vital signs, physical examination findings) there will be rules for non-escalation. Any AE that is considered related or probably related to nDoce is potentially a DLT. DLTs will, in addition, include the following: 1) Procedure-related events that require hospitalization or surgical intervention and some procedure-related events that require medical intervention; 2) All Grade 3-4 AE which are possibly related to study drug will be considered DLT except: a) Grade 3 nausea or Grade 3-4 vomiting and diarrhea that persist for less than 48 hours in patients who have not received optimal anti-emetic or anti-diarrhea prophylaxis; b) Grade 3 fatigue less than 5 days; c) Grade 3 laboratory abnormalities that are not clinically significant and return to normal (with or without intervention) within 48 hours; 3) Grade 3 thrombocytopenia with clinically significant hemorrhage; 4) Grade 2 toxicity that prevents further treatment or persists for at least 3 weeks; and 5) Any life-threatening event (unless there is a clear alternative explanation that the event is not related to the procedure or the investigational product itself).

Dose Adjustments/Modifications/Delays: Group 1: At any time during Induction or Maintenance, intravesical instillations will be withheld in weekly increments in the event of Grade 2 thrombocytopenia, anemia, neutropenia, hematuria (visible gross hematuria) or laboratory-confirmed urinary tract infections until the infection is resolved. The hematuria and abnormal CBC values must resolve to a maximum of Grade 1. Group 2: The study will evaluate one single nDoce intravesical instillation in each subject; therefore, there will be no dose adjustment or modification.

Duration of Therapy: At Visit 2, nDoce will be injected directly into the index tumor resection site followed by a single intravesical instillation. Group 1: Up to six Induction nDoce intravesical instillations and up to three Maintenance nDoce intravesical instillations will be administered. It is estimated that individual subject participation could last up to 33 weeks. Group 2: It is estimated that individual subject participation could last up to 74 days.

Other Assays or Procedures: Plasma samples will be collected to characterize the PK of nDoce. Group 1: Plasma samples will be collected at Visit 2 (prior to nDoce injection and at 1, 2, 4, 6, and 24 hours post-injection), at Visit 3 (prior to nDoce intravesical instillation and at 1, 2, 4, 6, and 24 hours post-intravesical instillation), at Visits 4-11 prior to the nDoce intravesical instillation, and at End of Treatment. Allowable windows will be: 10-minutes for the first 4-hour collections, 20-minutes at 6 hours post and 30-minutes at 24 hours post. Group 2: Plasma samples will be collected at Visit 2 (prior to nDoce injection and at 1, 2, 4, 6, and 24 hours post-injection) and at the End of Treatment Visit. Allowable windows will be: 10-minutes for the first 4-hour collections, 20-minutes at 6 hours post and 30-minutes at 24 hours post.

Study Schedule:

Screening (Visit 1): Groups 1 and 2 will complete the Screening visit (Visit 1). Assessments, visits, and other assays performed prior to consent to the study for nDoce injection will be performed and are not considered part of this study. The following procedures and assessments must be completed, documented and reviewed by the Investigator during the screening period, within 14 days prior to nDoce Visit 2 injection: written informed consent including comprehensive discussion of the study schedule, procedures and subject protocol requirements; complete medical history, including review of previous medical records and demographics; review and documentation of urothelial carcinoma diagnosis (diagnostic biopsy and/or imaging); review and documentation of previous treatments including surgical, chemotherapy and immunologic records; review and documentation of all concomitant prescription and non-prescription medications; comprehensive physical examination; ECOG (Eastern Cooperative Oncology Group) Performance Status; vital signs (blood pressure, heart rate, pulse and temperature), body weight and height; sample collection and processing for clinical laboratory assessments.

Trans Urethral Resection of Bladder Tumor (Visit 2): Groups 1 and 2 will complete Visit 2: Medical history confirmation (AE occurring prior to nDoce injection will be considered history); comprehensive physical examination (if not completed within the 14 days prior to Visit 2); ECOG Performance Status assessment; TURBT—confirmation of non-bladder perforation following TURBT must be documented and filed in the subject's study record and trans-urethral resection surface area to be recorded (<8 cm²). If subject does not qualify or bladder perforation is confirmed, subject will not proceed to nDoce injection and will be considered a Screen Fail. If continuation to nDoce injection is confirmed, bladder resection tissue sample will be collected and processed for histological assessments. Final review of inclusion and exclusion criteria and determination of eligibility will be conducted prior to nDoce injection.

nDoce Treatment—Injection & Intravesical Instillation (Visit 2 continued): Groups 1 and 2 will complete Visit 2: Baseline PK sample will be drawn prior to nDoce injection and may be collected prior to TURBT procedure; vital signs will be monitored and collected prior to nDoce injection; nDoce will be injected into the resected bladder tumor—start time of first injection and stop time of last injection will be recorded; vital signs will be monitored and collected post nDoce injection; nDoce will be instilled into the bladder intravesically—start time of instillation and time at end of void will be recorded; vital signs will be monitored and collected post nDoce intravesical instillate (catheter removed and instillate voided); collection of AEs (after nDoce injection) will be documented separately as treatment-emergent adverse events (TEAE), with a start date and time on or after direct injection administration; collection of concomitant medication; PK samples will be drawn at 1, 2, 4, 6, and 24 hours post nDoce injection stop time; subject will be provided a diary to record AE and concomitant medications until the next study visit.

Induction Period (Visits 3-8): Only Group 1 subjects will complete the Induction Period (Visits 3-8). Group 1 subjects will be assessed for recovery (TURBT resection site healing) at 4 weeks (1 month) after Visit 2, at which point the Investigator will evaluate subject symptoms, pathology (if available), gross hematuria or urinalysis findings. If the Investigator determines that the subject has not recovered at 4 weeks, evaluations will be repeated at least every 2 weeks until the subject has recovered and intravesical nDoce can be administered. The 3-month Induction period will consist of 6 weekly nDoce intravesical instillation treatments, followed by 6 weeks of rest. The following procedures will be performed: vital signs; directed physical exam; sample collection and processing for clinical laboratory assessments; non-clinically significant CBC and urinalysis will be confirmed prior to each intravesical instillation to rule out infection or DLT; PK samples will be drawn: Visit 3 only: PK samples will be drawn pre-nDoce instillation and at 1, 2, 4, 6, and 24 hours post nDoce instillation start time; visits 4-8: PK samples will be drawn pre-nDoce instillation; intravesical instillation of nDoce will be conducted; AE review; Concomitant medications review. At any time during the study, the Investigator may perform cytology, cystoscopy or biopsy (for positive or suspicious cytology or cystoscopic findings). As applicable, biopsy samples will be collected for histological and immunohistochemical assessments. Subject diary will be reviewed to confirm it is adequately completed. The subject will be questioned regarding discrepancies, missing entries and errors. Any discrepancy will be documented in the subject source documents by a delegated staff; and a new diary to be provided to the subject to record instillate void time and for daily completion to record adverse events and concomitant medications.

Maintenance Perion (Visits 9-11): Only Group 1 subjects will complete the Maintenance Period (Visits 9-11). Visit 9 will occur after subjects complete the last day of the 3-month Induction period. If biopsy is indicated, maintenance therapy with nDoce is to be withheld until the histopathology results are available. Initiation of maintenance therapy with nDoce may be delayed up to 3 weeks. For the purpose of this study, progression of disease will be defined as persistence, recurrence and/or progression of disease. At Visit 9, subjects with no recurrence or evidence of progression will proceed to a 3-month Maintenance period consisting of three nDoce intravesical instillations to be administered once weekly, in the first three weeks. The following procedures will be performed at Visits 9-11: Directed physical exam may be performed—Visit 9 only; ECOG—Visit 9 only; vital signs; sample collection and processing for clinical laboratory assessments); non-clinically significant CBC and urinalysis will be confirmed prior to each intravesical instillation to rule out infection or DLT; Intravesical nDoce instillations at Visits 9, 10 and 11; sample collection and processing for clinical laboratory assessments (Section 7.2.1)—Visit 9 only; PK samples will be drawn pre-nDoce instillation; cystoscopy, urine cytology—Visit 9 only; biopsy—performed for positive or suspicious cytology or cystoscopic findings, and as applicable, biopsy samples will be collected for histological and immunohistochemical assessments; AE review; concomitant medications review. Subject diary will be reviewed to confirm it is adequately completed. The subject will be questioned regarding discrepancies, missing entries and errors. Any discrepancy will be documented in the subject source documents by a delegated staff; and a new diary to be provided to the subject to record instillate void time and for daily completion to record adverse events and concomitant medications.

End of Treatment: The End of Treatment visit will be conducted after a subject completes any or all study treatment. The End of Treatment visit is planned for Month 6 for Group 1 and for 30 days after Visit 2 for Group 2. All diagnostic (to include, but not limited to biopsy, scan, or institution-required diagnostic testing) reports will be collected for study purposes at any time during the study and filed as part of the end of treatment procedures. Additional imaging may be performed; all resulting images and/or reports will be collected for the subject's record. At the visit, the following procedures will be performed: directed physical exam; ECOG; vital signs; 12-lead ECG; clinical laboratory sample collection; PK Sample collection (one sample collection only); cystoscopy and urine cytology; biopsy—performed for positive or suspicious cytology or cystoscopic findings, and as applicable, biopsy samples will be collected for histological and immunohistochemical assessments; AE collection; concomitant medications. Subject diary will be reviewed to confirm it is adequately completed. The subject will be questioned regarding discrepancies, missing entries and errors. Any discrepancy will be documented in the subject source documents.

A summary of the schedule of events is shown in Table 17, Table 18, and Table 19 below.

TABLE 17
Group 1 (NMIBC) Visits 1-8

| | Screening | | Induction (+/2 days)¹ | | | | | |
|---|---|---|---|---|---|---|---|---|
| | Visit 1 | | Visit 3 | Visit 4 | Visit 5 | Visit 6 | Visit 7 | Visit 8 |
| | Days | Treatment | Day 1 | Day 8 | Day 15 | Day 21 | Day 28 | Day 35 |
| Procedure | (−14-0) | Visit 2 | Week 1 | Week 2 | Week 3 | Week 4 | Week 5 | Week 6 |
| Informed Consent | X | | | | | | | |
| Medical and Surgical | X | | | | | | | |
| History² | | | | | | | | |
| Demographics | X | | | | | | | |
| Physical | X | X | X | X | X | X | X | X |
| ECOG | X | X | | | | | | |
| 12-Lead ECG | | | | | | | | |
| Vital Signs⁴ | X | X | X | X | X | X | X | X |
| Height and Weight | X | | | | | | | |
| PK Collection⁵ | | X | X | X | X | X | X | X |
| Laboratory Tests⁶ | X | | X | X | X | X | X | X |
| nDoce Direct | | X | | | | | | |
| Injection | | | | | | | | |
| nDoce | | X | X | X | X | X | X | X |
| Intravesical | | | | | | | | |
| TURBT ⁷ | | X | | | | | | |
| Cystoscopy ^{7, 8} | X | | X | | | | | |
| Cytology ^{7, 8} | X | | X | | | | | |
| Biopsy ^{7, 8} | X | | X ⁸ | | | | | |
| Diary Distribution | | X | X | X | X | X | X | X |
| Diary Collection | | | X | X | X | X | X | X |
| Adverse Events | | X | X | X | X | X | X | X |
| Concomitant therapy | X | X | X | X | X | X | X | X |

¹Group 1: Induction to begin post TURBT once resection site is healed.
²History includes all medical and surgical history prior to the first direct injection of nDoce;
3. Comprehensive physical examination required at screening and End of Treatment; targeted physical exam at all other visits, if required;
⁴Vitals will be performed prior to and post nDoce direct injection and intravesical instillations, per institution requirements;
⁵See Table 20 below for Visit 2 and Visit 3 detailed PK collection schedule;
⁶Laboratory testing to be performed and results reviewed prior to all Induction and Maintenance intravesical instillations.
⁷ Institution pathology and immuno-histochemical reports will be collected for any resection, cystectomy or other biopsy sampling to include, but not limited to, initial Visit 2 bladder resection, additional biopsies performed at any time during the study or early withdrawal cystectomy tissue or node samples;
⁸ Regularly scheduled or ad-hoc cystoscopy, urine cytology, and ‘for-cause’ biopsy to be performed prior to any nDoce instillation;
9. Maintenance doses to be administered to subjects classified as non-recurrence.

TABLE 18
Group 1 Schedule of Events (Visits 9 to End of Treatment)

| | | | | | Safety |
|---|---|---|---|---|---|
| | | | | | +30 days |
| | Maintenance⁹ (+/−3 days) | | | End of | after |
| | Visit 9 | Visit 10 | Visit 11 | Treatment | last study |
| | Day 85 | Day 92 | Day 99 | Month 6 | drug dose |
| | Week | Week | Week | (+/−7 | (+/−5 |
| Procedure | 13 | 14 | 15 | days) | days) |
| Physical | X | X | X | X | |
| Examination³ | | | | | |
| ECOG | X | | | X | |
| Vital Signs⁴ | X | X | X | X | |
| 12-Lead ECG | | | | X | |
| Laboratory | X | X | X | X | |
| Tests⁶ | | | | | |
| nDoce | X | X | X | | |
| Instillation | | | | | |
| PK | X | X | X | X | |
| Cystoscopy ^{7, 8} | X | | | X | |
| Cytology ^{7, 8} | X | | | X | |
| Biopsy ^{7, 8} | X | | | X | |
| Diary | X | X | X | | |
| Distribution | | | | | |
| Diary | X | X | X | X | |
| Collection | | | | | |
| Adverse Events | X | X | X | X | X |
| Concomitant | X | X | X | X | |
| Therapy | | | | | |

1. Group 1: Induction to begin post TURBT once resection site is healed.
2. History includes all medical and surgical history prior to the first direct injection of nDoce;
³Comprehensive physical examination required at screening and End of Treatment; targeted physical exam at all other visits, if required;
⁴Vitals will be performed prior to and post nDoce direct injection and intravesical instillations, per institution requirements;
5. See Table 20 below for Visit 2 and Visit 3 detailed PK collection schedule;
⁶Laboratory testing to be performed and results reviewed prior to all Induction and Maintenance intravesical instillations.
⁷ Institution pathology and immuno-histochemical reports will be collected for any resection, cystectomy or other biopsy sampling to include, but not limited to, initial Visit 2 bladder resection, additional biopsies performed at any time during the study or early withdrawal cystectomy tissue or node samples;
⁸ Regularly scheduled or ad-hoc cystoscopy, urine cytology, and ‘for-cause’ biopsy to be performed prior to any nDoce instillation;
⁹Maintenance doses to be administered to subjects classified as non-recurrence.

TABLE 19
Group 2 (MIBC) Schedule of Events

| | | | End of | Safety |
|---|---|---|---|---|
| | Screening | | Treatment | Visit |
| | Visit 1 | | Day 30 | 2 +30 days |
| | Days | Treatment | (+/−5 | (+/−5 |
| Procedure | (−14-0) | Visit 2 | days) | days) |
| Informed Consent | X | | | |
| Medical and Surgical | X | | | |
| History¹ | | | | |
| Demographics | X | | | |
| Physical | X | X | X | |
| ECOG | X | X | | |
| 12-Lead ECG | X | | | |
| Vital Signs³ | X | X | X | |
| Height and Weight | X | | | |
| PK Collection⁴ | | X | X | |
| Laboratory Tests | X | | X | |
| nDoce Direct | | X | | |
| nDoce | | X | X | |
| Intravesical | | | | |
| TURBT | | X | | |
| Cystoscopy ⁵ | X | | X | |
| Cytology ⁵ | X | | X | |
| Biopsy ⁵ | X | | X | |
| Diary Distribution | | X | X | |
| Diary Collection | | | X | X |
| Adverse Events | | X | X | X |
| Concomitant therapy | X | X | X | |

¹History includes all medical and surgical history prior to the first direct injection of nDoce;
2. Comprehensive physical examination required at screening and End of Treatment or early withdrawal; targeted physical exam at all other visits, if required;
³Vitals will be performed prior to and post nDoce direct injection and intravesical instillations, per institution requirements;
⁴See Table 20 below for detailed PK collection schedule;
⁵ Institution pathology and immuno-histochemical reports will be collected for any resection, cystectomy or other biopsy sampling to include, but not limited to, Visit 2 bladder resection, biopsy, cystectomy tissue or node samples performed at any time prior to the last study visit.

TABLE 20
Schedule of Pharmacokinetic Sample Collection
Pharmacokinetic Collection

| | | Post Dose | | | | |
|---|---|---|---|---|---|---|
| | | 1 Hour | 2 Hours | 4 Hours | 6 Hours | 24 Hours |
| | Pre-Dose | (+/−10 | (+/−10 | (+/−10 | (+/−20 | (+/−30 |
| Timepoint | 0 Hour | min) | min) | min) | min) | min) |
| Visit 2 ¹ | X ² | X | X | X | X | X |
| Visit 3 ³ | X ⁴ | X | X | X | X | X |

¹ Groups 1 and 2;
² PK sample collection within the 24 hours prior to the Visit 2 study drug direct injection;
³ Group 1 only;
⁴ PK sample collection within the 24 hours prior to the Visit 3 study drug intravesical instillation.

The results of this study will demonstrate the effectiveness of a method of treatment for treating bladder cancer or inhibiting the recurrence of bladder cancer in a subject comprising: directly injecting an effective amount of a first composition comprising taxane particles into a bladder tumor surgical resection site (following surgical resection of the bladder tumor); followed by intravesical instillation of an effective amount of a second composition comprising taxane particles into the bladder of the subject.

1. A method of treating bladder cancer or inhibiting the recurrence of bladder cancer in a subject, the method comprising: directly injecting an effective amount of a first composition comprising taxane particles into one or more bladder tumor surgical resection sites, wherein the injecting is done following surgical resection of one or more bladder tumors of the subject, wherein the taxane particles have a mean particle size (number) of from 0.1 microns to 5 microns, thereby treating or inhibiting the recurrence of the bladder cancer.

2. The method of claim 1, wherein the method further comprises: a first (initial) instilling via intravesical instillation of an effective amount of a second composition comprising a taxane solution or taxane particles having a mean particle size (number) of from 0.1 microns to 5 microns into the bladder of the subject after injecting the first composition.

3. The method of claim 2, wherein the method further comprises: instilling via intravesical instillation of an effective amount of the second composition into the bladder of the subject an additional 1 to 14 times after the first (initial) instilling.

4. The method of claim 3, wherein the instillations are separated by periodic intervals.

5. The method of claim 1, wherein the taxane particles of the first composition have a mean particle size (number) of from 0.1 microns to 1.5 microns, wherein the second composition comprises taxane particles, and wherein the taxane particles of the second composition have a mean particle size (number) of from 0.1 microns to 1.5 microns.

6. The method of claim 1, wherein the taxane particles comprise at least 95% of the taxane.

7. The method of claim 1, wherein the taxane particles of the first composition are docetaxel particles, wherein the second composition comprises taxane particles, and wherein the taxane particles of the second composition are docetaxel particles.

8. The method of claim 7, wherein the docetaxel particles have a specific surface area (SSA) of at least 18 m²/g.

9. The method of claim 7, wherein the docetaxel particles have a bulk density (not-tapped) of 0.05 g/cm³ to 0.15 g/cm³.

10. The method of claim 2, wherein the second composition comprises a taxane solution, and wherein the taxane solution is docetaxel solution.

11. The method of claim 1, wherein the first composition and/or the second composition exclude albumin.

12. The method of claim 1, wherein the first composition further comprises a liquid carrier, wherein the first composition comprises a suspension of the taxane particles dispersed in the liquid carrier, wherein the second composition comprises taxane particles, wherein the second composition further comprises a liquid carrier, and wherein the second composition comprises a suspension of the taxane particles dispersed in the liquid carrier.

13. The method of claim 12, wherein the first composition further comprises a diluent, wherein the liquid carrier and the diluent form a mixture, wherein the first composition is a suspension of the taxane particles dispersed in the liquid carrier/diluent mixture, wherein the second composition comprises taxane particles, wherein the second composition further comprises a diluent, wherein the liquid carrier and the diluent form a mixture, and wherein the second composition is a suspension of the taxane particles dispersed in the liquid carrier/diluent mixture.

14. The method of claim 7, wherein the concentration of the docetaxel particles in the first composition is about 1 mg/mL to about 4 mg/mL.

15. The method of claim 7, wherein the second composition comprises docetaxel particles, wherein the concentration of the docetaxel particles in the second composition is about 1 mg/mL to about 15 mg/mL.

16. The method of claim 1, wherein the bladder cancer is non-muscle invasive bladder cancer (NMIBC) or muscle invasive bladder cancer (MIBC).

17. A method for inhibiting the recurrence of bladder cancer in a subject who has had one or more bladder tumors surgically resected, the method comprising:
(a) following surgical resection of the one or more bladder tumors, directly injecting an effective amount of a first composition comprising taxane particles into the resection site(s), wherein the taxane particles have a mean particle size (number) of from 0.1 microns to 5 microns;
(b) a first (initial) instilling via intravesical instillation of an effective amount of a second composition comprising a taxane solution or taxane particles having a mean particle size (number) of from 0.1 microns to 5 microns into the bladder of the subject after injecting the first composition; and
(c) instilling via intravesical instillation of an effective amount of the second composition into the bladder of the subject an additional 1-14 times after the first (initial) instilling;
wherein the bladder cancer does not recur in the subject for at least 3 months, or at least 6 months, or at least 12 months after the surgical resection of the one or more tumors, thereby inhibiting the recurrence of the bladder cancer.

18-30. (canceled)

31. A method of treating bladder cancer in a subject, the method comprising:
(a) administering a first administration (first cycle) of an effective amount of a composition comprising taxane particles to a bladder tumor of the subject via intratumoral injection, wherein the taxane particles have a mean particle size (number) of from 0.1 microns to 5 microns,
(b) optionally, administering a second administration (second cycle) of an effective amount of the composition to the bladder tumor via intratumoral injection within a periodic interval following the first administration in (a), and
(c) optionally, administering a third administration (third cycle) of an effective amount of the composition to the bladder tumor via intratumoral injection within a periodic interval following the second administration in (b),
thereby treating the bladder cancer.
